SEQUENCE LISTING (130 sequences)

SEQ ID NO: 1 - DNA, 8699 bp, Homo sapiens
  misc_feature (2697)..(2697): n = unknown
gcttcattgttccacgcattacgtctcaccagtttagtcaatatggattaaatatgagagtgtggcaattcgcaaactctatctgaggaggaaaatcggataaaaaatgttatgaaaaataaagcaatttgaagcctctgacttcagcaacttcaccactaatgaaatgatgtaaccctcattggcctcaaatttagttttcacggggcatctgcagggttccaaagtgagaccaggtgaattcaatgtgcatgcacttcccaagtgtccacttgtattctgtttctttacttctgtttacagaaagtagacacatattcagtcttagtaccagtgtagggagcgctttccatgagatggataccagaaaaaaatggcaaacatgggatccgttaatataaaaattagccacgatgtatatatatatatgtgtgtgtgtgtgtgtgtgtgtgtgtacacacgcgcgcgcatgtgtgagttgaatagcagagttggagtgggtttctatccacatgtacctgcacctgcaggtattctcaggtgccataatcaactgtaggaccctaaaggaaataagagtctcccctcaacccctgaagagtgtttgggttcaccgtgtgtccaatgattctgtgcctcttgagctccaggaaagggctccctggtgatgcatgagatcttttcttggagtctctctgcagagttcactgggtttcctaaaggcaattcactatttcaaaagatggtgtgaggagcatgtggtgtccctaaaggagaattctgagccagggcacaaccactttatactgagctggatacactggtaggaatatactctgtcagctcagatagaaacctccctgcatggttggggctgggctgcagggggcgctccggatacacccagcacaggctcccgccccagagcaggtgcacaggaggctggggagaggttcctcccagggcctgggacttcctttaaaaatatctaaaataagtatttcacaaagactgctgaagtttgtataaatatctattcaattgtgagcatttatcaaactggatgttgtaatgagaaccacttttacaatggggatttcaaactctgctggaggtcaggaagagatcctttcttataaataaatgcaatttttggataaacacagtcattccctaaataacgcattcacatattatggtctagaaatgatgcaagttgaccctgagacagtcaaatgtggtttcaaagtgaggtgctgtccttgaggagcttgttctccagtgggggaagctctgtcaacacagagttcagggatgtgtaggggacacatggcctctaacaggattacggcttgaaccctcagcttctacagttgtgtcacccatgtgtctgtttctcatactgggtcaggaattgggctattaaatagcatccttcatgaatatgcaattaactgaggtgactatagtatctctgtaccctgaaagcatcacccaacaacaacatccctccttgggagaatcccctagagcacagctcctcaccatggactggacctggagcatcctcttcttggtggcagcagcaacaggtaaggggctccccagtctcggggttgaggcagaaaccaggccactcaagtgaggctttacccacccctgtgtcctctccacaggtacctactcccaggtgcagctggtgcagtctggccatgaggtgaagcagcctggggcctcagtgaaggtctcctgcaagtcttctggttacaccttcaccatctatggtatgaattgggtatgatagacccctggacagggctttgagtggatgtgatggatcatcacctacactgggaacccaacgtatacccacggcttcacaggatggtttgtcttctccatggacacgtctgtcagcacggcgtgtcttcagatcagcagcctaaaggctgaggacacggccgagtattactgtgcgaagtacacagtgtggaaacccacatcccgagagtttcagaaagcctgaggaaggaggcagctgtgctgagctgaggcagtggtacagcagttttctgaacttccatagtatctcattttgcattgagttccgctttaatattagccaagaatatgggatagacgggtgctcctaagagatccttaacttgcccattttgatgggttttcccaaagacgtgagaagccacttttttcgcaaagcatcccaaagccatgccctgctccagaaacacgtgtatccatttcctggtctttgattaactgacaaactctcatcagcgcacctgggctaatttcacatcaggtagaaatatgtgctttaaagcaaggctaacgttgtaatagcaattcctgcttaataaccttcagcattgttgttgtgtgctccatcaactaattacgttagttcaaggttctcaatgggagtttctaataaatataagggatgtatagaagttcccctaattaaaacaattgtgaagacaacctcagtgttcaaccatatttcaacccttcaccacaaaggaactttcatctctcctggaagttgggttcattttcaaattagtttttttattttaatatctcaagattattgtatgtgactattttagcagaaagtgaattatgggaacttgaactaaccaactgaaaatacattcagaactaattaaacangatgccagaatgtgattggctccaggcattttaaattcaacaggttatgtaaccaggctttaaatttgcacatcttcgtgttaccttcatgacacagtcaactcccattatgtaagaaatggtgagtgcattcccaagggtcttgcacagttataaaaatagacttgatgaggtgaggagttgtttaaattcccctctgaagaagcagcatcaacccaacaaaccactctcttccctctgtgactagagctctgtcacaggccacatggacctaaatccttgatggagattacaggactacgtaaattggactgatcgtttttatgctgttaaattaataggtgagtctgcactccagcctgggcaacagaataatcttgtctgtaaaatacaaaagaaagataaattaatagatactgactttgacatttcggataataatattttcataaaccgaatttaattatacccacattgttacctacaccttcactgaaaagttcctagttatgttgagttccatcaacactccacatgttcaaatctggacatccaagagagtctagagaataaaacgcaatgagggcagtgaaacttgcgtatattcagcacctcttaactcaggaggactcaatacaccctggaacactctgcttttctgaatggctcacaatgactccagctcactctccaacctccgcaaacatctggcctctgtttgccctaagttcacgctctgctcttagtctatgttctgaagtctttgtagaggtgaaaatgagctgtcagatggatcttccttctcactgcaacatggaatttgctatttcacttaatgaccactctttccacaatggttgatttcttttggcctgttcattactggtgattttcaagggaatctcagttgaatctttactgttttgcattttgtctccatgacaatgttgggaagtttttcttctagcagcataacatgatctagtgacctgacacatttgcagcaaacaatacctacaaattcagaagctctttggttttctttccacgaaatataattcttgctcttctgtgtatgagcacatcctagcatccctgtacacacccaggtagatgtctacacgccgatgaaatattccctgtaaataaaaaaagtatctcagtttctctcaatgttcataattctcctgagggtgaggaaggtacttctgggtctgctcaaacaaatggcccagagaccacctggtaggtaggtaaggagctcacctcgctctggatattgagtctgtctctttccctctgtcgtctcatagaagnccagcccacttgttcagctcctaagaagagagcccaggtttatccagattatacaacacaaccagcttctgatgactctcctgttacaacatccatggagatattttgtgtattatataattcaccaaactaatgtgaaatgcccaagttgcaatactgcacaccctagggtatgttcttgcaattcagcggaggagaaattctttcagagacagatggatctgaattggtaaatatgtgggtacgaattctgggcttgagtgtcattgtccagccatgtttcacaggtgtgacctgtcagggaagaaccagagttccttgttctctcagagggtagagctcacagaggtcctctctggttcccaggaaaggtaatttcactaatcttggtgatgagactatcctccagtgctgatgtactatagagttttcatctgaagctgtcactgctatccccaatgtacatcttttcacacagaaatgtttagaggtcaggccatattctcagggttacacattgagaaggatggagatatattctactaccttctcctgagatctcacacacaatctcaaatttcaaaaggtctcagaagggcagctctcaggtactatttaaaaataacccacttcctgggacaggtagcatccttctaaccatgatggatgttctgaactacagtacacattgcatggatccaggtttgtctcaattcactgtgattattacactcagcagctgtttcaatatgtctgaaggggtaaatgacaatttaggtgacctgggtgtatggttggtgttatatgaatctttaaatgtagaacagtattaactgtattccaaaatctgtctttgatccatgatcacacttgtctcccagaccagctccttcagcacatttcctacctggaagaagaggactctgggtttggtgaggggaggccacaggaagagaactgagttctcagagggcacagccagcatacacctcccagggtgagcccaaaagactggggcctccctcatccctttttacctatccatacaaaggcaccacccacatgcaaatcctcacttaggcacccacaggaaatgactacacatttccttaaattcagggtccagctcacatgggaagtgctttctgagagtcatggacctcctgcacaagaacatgaaacacctgtggttcttcctcctcctggtggcagctcccagatgtgagtgtctcaggaatgcggatatgaagatatgagatgctgcctctgatcccagggctcactgtgggtttttctgttcacaggggtcctgtcccaggtgcagctacagcagtggggcgcaggactgttgaagccttcggagaccctgtccctcacctgcggtgtttatggtgggtccttcagtggttactactggagctggatccgccagcccccagggaaggggctggagtggattggggaaatcaatcatagtggaagcaccaactacaacccgtccctcaagagtcgagtcaccatatcagtagacacgtccaagaagcagctctccctgaagttgagctctgtgaacgccgcggacacggctgtgtattactgtgcgagagttattactagggcgagtcctggcacagacgggaggtacggtatggacgtctggggccaagggaccacggtcaccgtctcctcaggtgagaatggccactctagggcctctgttctctgctactgcctgtggggtttcctgagcattgcaggttggtcctcggggcatgttccgaggggacctgggcggactggccaggaggggacgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgacttaaaaatgtgagagattttcaagtatattaatttttttaactgtccaagtatttgaaattcttatcatttgattaacacccatgagtgatatgtgtctggaattgaggccaaagcaagctcagctaagaaatactagcacagtgctgtcggccccgatgcgggactgcgttttgaccatcataaatcaagtttatttttttaattaattgagcgaagctggaagcagatgatgaattagagtcaagatggctgcatgggggtctccggcacccacagcaggtggcaggaagcaggtcaccgcgagagtctattttaggaagcaaaaaaacacaattggtaaatttatcacttctggttgtgaagaggtggttttgcccaggcccagatctgaaagtgctctactgagcaaaacaacacctggacaatttgcgtttctaaaataaggcgaggctgaccgaaactgaaaaggctttttttaactatctgaatttcatttccaatcttagcttatcaactgctagtttgtgcaaacagcatatcaacttctaaactgcattcatttttaaagtaagatgtttaagaaattaaacagtcttagggagagtttatgactgtattcaaaaagttttttaaattagcttgttatcccttcatgtgataattaatctcaaatactttttcgatacctcagagcattattttcataatgactgtgttcacaatctttttaggttaactcgttttctctttgtgattaaggagaaacactttgatattctgatagagtggccttcattttagtatttttcaagaccacttttcaactactcactttaggataagttttaggaaaaatgtgcatcattatcctgaattatttcagttaagcatgttagttggtggcataagagaaaactcaatcagatagtgctgaagacaggactgtggagacaccttagaaggacagattctgttccgaatcaccgatgcggcgtcagcaggactggcctagcggaggctctgggagggtggctgccaggcccggcctgggctctgggtctccccggactacccagagctgggatgcgtggcttctgctgccgggccgactggctgctcaggccccagcccttgttaatggacttggaggaatgattccatgccaaagctttgcaaggctcgcagtgaccaggcgcccgacatggtaagagacaggcagccgccgctgctgcatttgcttctcttaaaactttgtatttgacgtcttatttccactagaaggggaactggtcttaattgcttgatgaagagcaggagactcatttatgtgagtcttttgagtgaccattgtctgggtcactcccatttaactttccctaaagcccatttgaaggagaggtcgcacgagctgctccacaacctctgaatggggatggcatgggtaatgatgcttgagaacataccaagtcccactggcatcgcccttgtctaagtcattgactgtaggtcatcatcgcacccttgaaagtagcccatgccttccaaagcgatttatggtaaatggcagaattttaagtggcaaattcagataaaatgcatttcttggttgtttccaatgatgactgttatctagagggaatttaaaggcaggggtttactgcagactcagaagggaggggatgctccgggaaggtggaggctctgagcatctcaataccctcctcttggtgcagaagatatgctgccacttctagagcaaggggacctgctcatttttatcacagcacaggctcctaaattcttggtctcattctcaagatgttttaatgactttaaagcagcaaagaaatattccacccaggtagtggagggtggtaatgattggtaatgctttggaaccaaaacccaggtggcgctggggcaggactgcagggaactggggtatcaagtagagggagacaaaagatggaagccagcctggctgtgcaggaacccggcaatgagatggctttagctgagacaagcaggtctggtgggctgaccatttctggccatgacaactccatccagttttcagaaatggactcagatgggcaaaactgacctaagttgacctagactaaacaaggccgaactgggctgagctgagctgaactgggctgagttgaactgggctgagctgagctgagctgagctgggctaagttgcaccaggtgagctgagttgagctgggcttggctgcattaaggtgggctgagctgggcagggctgggctgaattgagctgggctgggctgagctaggctgggctgggc

SEQ ID NO: 2 - DNA, 338 bp, Homo sapiens
  misc_feature (54)..(54): n = unknown
taagtgaatcctggtgtgtctgaactcaagtgattgttacattaagctgctgtnccaatctgtttcctcacctgggaaaagagganccaggacatagtgagttgaggccccaggaagataactgaattctcagagggcacagccagcatcctcctcccagggagagtctaaaagactggggcctccctcatcccttttcacctgtccatacagaggcaccacccacatgcaaatctcacttaggcacccacagaaaaccaccacacatttccttaaattcagggtcctgctcacatgggaaatactttctgagagtcctggacctcctgtgcaagaac

SEQ ID NO: 3 - DNA, 339 bp, Homo sapiens
taagtgaatcctggtgtgtctgaactcacatgattgttacattaagctgctgttgcaatctgtttcctcacctgggaaaagaggagccaggacatagtgagttgaggccccaggaagataactgaattctcagagggcacaaccagcatcctccttgccagggagagcctaaaagactggggcctccctcatcccttttcacctctccatacagaggcaccacccacatgcaaatctcacttaggcacccaagggaaaccatcacacatttccttaaattcagggtcctgctcacatgggaaatactttctgagagctctggacctcctgtgcaagaac

SEQ ID NO: 4 - DNA, 338 bp, Homo sapiens
  misc_feature (106)..(106): n = unknown
attccaaaatctgtctttgatccatgatcacacttgtctcccagaccagctccttcagcacatttcctacctggaagaagaggactctgggtttggtgaggggagnccacaggaagagaactgagttctcagagggcacagccagcatacacctcccagggtgagcccaaaagactggggcctccctcatccctttttacctatccatacaaaggcaccacccacatgcaaatcctcacttaggcacccacaggaaatgactacacatttccttaaattcagggtccagctcacatgggaagtgctttctgagagtcatggacctcctgcacaagaac

SEQ ID NO: 5 - DNA, 339 bp, Homo sapiens
attccaaaatctgtccctgatccaagatcacactgatctcccagagcagcatcttcagcacatttccctacctggaagaagaggactatgggcttggtaaggggaggccacaggaagagaactgagttctcagagggcacagccagcttcctactcccagggcaagcccaaaagactggggcctccctcctcccttttcacctgtccatacaaagtcaccgcccacatgcaaatcctcacttaggcacctacaggaaaccagcacacatttccttaaatttgggatccagctcacatgggaaatactttctgagactcatgggcctcctgcacaagaac

SEQ ID NO: 6 - DNA, 338 bp, Homo sapiens
  misc_feature (16)..(16): n = unknown
atcccaaaatctgtcnttgatccaggatcacactcatctctcagaccagctccttcagcacatctctttacctggaagaagaggactctgggcttggagaggggagcccccaagaagagaactgagttctcaaagggcacagccagcattctcctcccagggtgagctcaaaagactggcgcctctctcatcccttttcactgctccgtacaaacgcaccacccccatgcaaatcctcacttaggcgcccacaggaagccaccacacatttccttaaattcaggtccaactcataagggaaatgctttctgagagtcatggatctcatgtgcaagaaa

SEQ ID NO: 7 - DNA, 334 bp, Artificial Sequence
  Consensus sequence for human immunoglobulin heavy chain promotor for VH4 alleles
atccaaaatctgtctctgatccaagatcacacttatctccagagcagctcttcagcacatttccttacctggaagaagaggactctgggcttggtgaggggaggccccaggaagagaactgagttctcagagggcacagccagcatcctcctcccaggggagcccaaaagactggggcctccctcatcccttttcaccttccatacaaaggcaccacccacatgcaaatcctcacttaggcacccacaggaaaccaccacacatttccttaaattcagggtccagctcacatgggaaatactttctgagagtcatggacctcctgtgcaagaac

SEQ ID NO: 8 - DNA, 3693 bp, Artificial Sequence
  Plasmid pME18Sasfp499
aagcttggctgtggaatgtgtgtcagttagggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgatcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctctgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcgctcgagatgtatccttccatcaaggaaaccatgcgcgttcagctttctatggagggtagtgttaactaccacgccttcaagtgcactggaaaaggagagggaaaaccatacgaaggcacccaaagcctgaatattacaataactgaaggaggtcctctgccatttgcttttgacattctgtcacacgcctttcagtatggcatcaaggtcttcgccaagtaccccaaagaaattcctgacttctttaagcagtctctacctggtggtttttcttgggaaagagtaagcacctatgaagatggaggagtgctttcagctacccaagaaacaagtttgcagggtgattgcatcatctgcaaagttaaagtccttggcaccaattttcccgcaaacggtccagtgatgcaaaagaagacctgtggatgggagccatcaactgaaacagtcatcccacgagatggtggacttctgcttcgcgatacccccgcacttatgctggctgacggaggtcatctttcttgcttcatggaaacaacttacaagtcgaagaaagaggtaaagcttccagaacttcactttcatcatttgcgtatggaaaagctgaacataagtgacgattggaagaccgttgagcagcacgagtctgtggtggctagctactcccaagtgccttcgaaattaggacataacgcggccgctgattataaagatgatgatgataaagattataaagatgatgatgataaagattataaagatgatgatgataaagattataaagatgatgatgataaataataaactagtctagagaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccgggtaccgagctcgaattaattcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgc

SEQ ID NO: 9 - DNA, 581 bp, Homo sapiens
cggccccgatgcgggactgcgttttgaccatcataaatcaagtttatttttttaattaattgagcgaagctggaagcagatgatgaattagagtcaagatggctgcatgggggtctccggcacccacagcaggtggcaggaagcaggtcaccgcgagagtctattttaggaagcaaaaaaacacaattggtaaatttatcacttctggttgtgaagaggtggttttgcccaggcccagatctgaaagtgctctactgagcaaaacaacacctggacaatttgcgtttctaaaataaggcgaggctgaccgaaactgaaaaggctttttttaactatctgaatttcatttccaatcttagcttatcaactgctagtttgtgcaaacagcatatcaacttctaaactgcattcatttttaaagtaagatgtttaagaaattaaacagtcttagggagagtttatgactgtattcaaaaagttttttaaattagcttgttatcccttcatgtgtaattaatctcaaatactttttcgatacctcagagcattattttcataatgactgtgttcacaatcttttt

SEQ ID NO: 10 - DNA, 582 bp, Homo sapiens
cggccccgatgcgggactgcgttttgaccatcataaatcaagtttatttttttaattaattgagcgaagctggaagcagatgatgaattagagtcaagatggctgcatgggggtctccggcacccacagcaggtggcaggaagcaggtcaccgcgagagtctattttaggaagcaaaaaaacacaattggtaaatttatcacttctggttgtgaagaggtggttttgcccaggcccagatctgaaagtgctctactgagcaaaacaacacctggacaatttgcgtttctaaaataaggcgaggctgaccgaaactgaaaaggctttttttaactatctgaatttcatttccaatcttagcttatcaactgctagtttgtgcaaacagcatatcaacttctaaactgcattcatttttaaagtaagatgtttaagaaattaaacagtcttagggagagtttatgactgtattcaaaaagttttttaaattagcttgttatcccttcatgtgataattaatctcaaatactttttcgatacctcagagcattattttcataatgactgtgttcacaatcttttt

SEQ ID NO: 11 - DNA, 666 bp, Ovis aries
ctgcgaataccgagacggggcctctcaaagccacccctgatagtctggaaaattgaaactttaaaaagagagatgtttaaagtattttaaatttttatcatttaattaacaactgcgaatcatggctttggagagttgagtaagagtttggctgaaaagtactaactaggttccatcggccctcggccccaattcagggctgttttgagaataataaattcagcttatttttttaatgtaattggtggtgccgagttagtcaagatggccacgggccagactgaccacctgcagcaggtggcaggaagcatgtccacttgagagtctgtttttggaagcaagaaaaaacagttggtaaatttatcgcttctggtttccaaaaggtggtttgcggctggttttgcccagccccacagaaccgaaagtgttccactgagcacaacagcacctggctaatttgcatttctaaaataaggcgcagatgctgaccgaaactggaaggttcctcttctaactatttgagttaacttcagctttagcttatcaactgctcacttatcttcattttcaaagtcgatgtttaagaaagccacctgtctcgggtgcactgtctcggtgcattgctgcactctctgatgagccgtccttcaaggtggttgagctgag

SEQ ID NO: 12 - DNA, 1067 bp, Mus musculus
ctagatactgagttctggttctaataactggctcctgtactgatggatgggtcctgactagtcattgggccctgatcctcaacattgacttcaaaacctgaactctagccccatgcctcattcacattaggatgatccctacaggggattcctgcagaagattccagaatccccacaacactgttcacacactgggctgcaactgggacagtgacccttttgactcataggacttgccaggcacagaggcacagaatggagacaaagcaagcccaggaccctggagatggagcctctggtggggtctacagatgtggggtcagcatcgtagggaggtttgcagggcaggtgtggggcagggcagaggtagtcatgcttatagatactatttttctctcctctggagcctcctttgtctatcacctgctgtcctgggatctctatctggggtcaacaatgtttgcagtacaggtgtgggggtagggcagggatgctcacattagcaacttgtttttctctcttctgaagtctctgttgtctatcacctgctgaaacattcaaagcagctctcagctgagggcagctgagtcatcctgagcctgtctcagcacaggtgccccaaaccagagctactgttctgagaatcacatcacactggaccaggccaggtgggcctgggacatggatgaggggtgggagccaggggagcctgccaggggctgaggaggccccaacccccactacccaaggccatccacacctgtgccttagtgaggccatgttctgtcccaatgagaacaagtccaattaagattaagtatggtcttcccaggactatccagagctaaggggtgtcagccagggacaacccagaccagcctgaggtcagccagcatcacccaaggccacacagctattctggctagaggactagatagctagctcatcgaggccctggagatgcagaatggaagagtttatccctgccagacagggctcatcagaaaggcaggtatctcactacacatgacctccctgaatatttcccagagtccagttggttctag

SEQ ID NO: 13 - DNA, 221 bp, Mus musculus
agtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgagg

SEQ ID NO: 14 - DNA, 808 bp, Mus musculus
agctcaaaccagcttaggctacacagagaaactatctaaaaaataattactaactacttaataggagattggatgttaagatctggtcactaagaggcagaattgagattcgaaccagtattttctacctggtatgttttaaattgcagtaaggatctaagtgtagatatataataataagattctattgatctctgcaacaacagagagtgttagatttgtttggaaaaaaatattatcagccaacatcttctaccatttcagtatagcacagagtacccacccatatctccccacccatcccccataccagactggttattgattttcatggtgactggcctgagaagattaaaaaaagtaatgctaccttattgggagtgtcccatggaccaagatagcaactgtcatagctaccgtcacactgctttgatcaagaagaccctttgaggaactgaaaacagaaccttaggcacatctgttgctttcgctcccatcctcctccaacagcctgggtggtgcactccacaccctttcaagtttccaaagcctcatacacctgctccctaccccagcacctggccaaggctgtatccagcactgggatgaaaatgataccccacctccatcttgtttgatattactctatctcaagccccaggttagtccccagtcccaatgcttttgcacagtcaaaactcaacttggaataatcagtatccttgaagagttctgatatggtcactgggcccatataccatgtaagacatgtggaaaagatgtttcatggggcccagacacgttctag

SEQ ID NO: 15 - DNA, 60 bp, Mus musculus
aagcagccctcaggcagaggataaaagctcacactaactgagaagctccatcctcttctc

SEQ ID NO: 16 - DNA, 60 bp, Mus musculus
aattaggccaccctcatcacatgaaaaccagcccagagtgactctagcagtgggatcctg

SEQ ID NO: 17 - DNA, 60 bp, Mus musculus
catgtgcgactgtgatgattaatatagggatatccacaccaaacatcatatgagccctat

SEQ ID NO: 18 - DNA, 60 bp, Mus musculus
aacatgagtctgtgattataaatacagagatatccataccaaacaacttatgagcactgt

SEQ ID NO: 19 - DNA, 338 bp, Homo sapiens; identical to SEQ ID NO: 6
  misc_feature (16)..(16): n = unknown

SEQ ID NO: 20 - DNA, 255 bp, Homo sapiens
agatataactatattttcctgaatgatggaattactaccagtctcccccaggacacttcatctgccctgagcccagcctctcctcagatgtcccacccagagcttgctatatagtgggggacatgcaaatagggccctccctctactgatgaaaaccagcccagccctgaccctgcagctctgggagaggagcccagcactagaagtcggcggtgtttccattcggtgatcagcactgaacacagaggactcacc

SEQ ID NO: 21 - DNA, 238 bp, Homo sapiens
cagtagaaatgctaataagaattaattgtttatgaagtgtaatcactctgggacacagcccactcagaggcatcccttccagaacccgctatatagtaggagacatgcaaatagggccctccctctgctgatgaaaaccagcccagccctgaccctgcagctctgggagaggagccccagccctgagattcccaggtgtttccattcagtgatcagcactgaacacagaggactcacc

SEQ ID NO: 22 - DNA, 244 bp, Homo sapiens
gatgggtaggggatgcgtgtcctctaacaggattacgtcttgaaccctcagcttctacaattgtgtcgtccatgtgtcatgtatttgctctttctcatcctgggtcaggaattgggctattaaatagcatccttcatgaatatgcaaataactgaggtgaatatagatatctgtgtgccctgagagcatcacccaaaaaccacacccctccttgggagaatcccctagatcacagctcctcacc

SEQ ID NO: 23 - protein, 36 aa, Homo sapiens
PNKGSTTSGTTRLLSGHTCFTLTGLLGTLVTMGLLT

SEQ ID NO: 24 - DNA, 111 bp, Homo sapiens
ccaaataaaggaagtggaaccacttcaggtactacccgtcttctatctgggcacacgtgtttcacgttgacaggtttgcttgggacgctagtaaccatgggcttgctgact

SEQ ID NO: 25 - protein, 33 aa, Sus scrofa
CRTNYGYSAAPSLHLPPGSLLASLVPLLLLSLP

SEQ ID NO: 26 - DNA, 99 bp, Sus scrofa
tgccggacgaattacggctactcagccgcccccagcctccacctcccgccgggctcgctgctggcctccctcgtgcccctcctcctcctcagtcttccg

SEQ ID NO: 27 - protein, 30 aa, Rattus rattus
ASSQSYRMTWNILYTLLISMTTLFQISTKE

SEQ ID NO: 28 - DNA, 90 bp, Rattus rattus
gcatcgtctcagagctacaggatgacctggaacatactctatacactgttaatcagcatgactactttattccaaatatctaccaaggag

SEQ ID NO: 29 - protein, 47 aa, Mus musculus
SSNKSISVYRDKLVKCGGISLLVQNTSWMLLLLLSLSLLQALDFISL

SEQ ID NO: 30 - DNA, 135 bp, Mus musculus
agctccaataaaagtatcagtgtgtatagagacaagctggtcaagtgtggcggcataagcctgctggttcagaacacatcctggatgctgctgctgctgctttccctctccctcctccaagccctggacttcatt

SEQ ID NO: 31 - protein, 37 aa, Mus musculus
PEDPPDSKNTLVLFGAGFGAVITVVVIVVIIKCFCKH

SEQ ID NO: 32 - DNA, 111 bp, Mus musculus
ccagaagaccctcctgatagcaagaacacacttgtgctctttggggcaggattcggcgcagtaataacagtcgtcgtcatcgttgtcatcatcaaatgcttctgtaagcac

SEQ ID NO: 33 - protein, 25 aa, Homo sapiens
MGIQGGSVLFGLLLVLAVFCHSGHS

SEQ ID NO: 34 - DNA, 75 bp, Homo sapiens
atgggaatccaaggagggtctgtcctgttcgggctgctgctcgtcctggctgtcttctgccattcaggtcatagc

SEQ ID NO: 35 - DNA, 39 bp, Artificial Sequence
  Oligonucleotide primer
ccggaattcaatttgagattgtgtgtgagatctcaggag

SEQ ID NO: 36 - DNA, 39 bp, Artificial Sequence
  Oligonucleotide primer
ccggaattcatagacagcgcaggtgagggacagggtctc

SEQ ID NO: 37 - DNA, 39 bp, Artificial Sequence
  Oligonucleotide primer
ccggaattcctgagaactcagttctcttcctgtggcctc

SEQ ID NO: 38 - DNA, 39 bp, Artificial Sequence; identical to SEQ ID NO: 35
  Oligonucleotide primer

SEQ ID NO: 39 - DNA, 39 bp, Artificial Sequence
  Oligonucleotide primer
cccaagctttcctgttacaacatccatggagatattttg

SEQ ID NO: 40 - DNA, 39 bp, Artificial Sequence
  Oligonucleotide primer
ccggaattctgaattgcaagaacataccctagggtgtgc

SEQ ID NO: 41 - DNA, 40 bp, Artificial Sequence
  Oligonucleotide primer
ccggaattctagggcaaacagaggccagatgtttgaggag

SEQ ID NO: 42 - DNA, 39 bp, Artificial Sequence
  Oligonucleotide primer
ccggaattcaatttaacagcataaaaacgatcagtccaa

SEQ ID NO: 43 - DNA, 39 bp, Artificial Sequence
  Oligonucleotide primer
ccggaattccgtgtttctggagcagggcatggctttggg

SEQ ID NO: 44 - DNA, 39 bp, Artificial Sequence
  Oligonucleotide primer
ccggaattcgttgggttcccagtgtaggtgatgatccat

SEQ ID NO: 45 - DNA, 39 bp, Artificial Sequence
  Oligonucleotide primer
ccggaattctcccaggaagtgggttatttttaaatagta

SEQ ID NO: 46 - DNA, 39 bp, Artificial Sequence
  Oligonucleotide primer
ccggaattcactatagtcacctcagttaattgcatattc

SEQ ID NO: 47 - DNA, 39 bp, Artificial Sequence
  Oligonucleotide primer
cccaagcttgacttcctttaaaaatatctaaaataagta

SEQ ID NO: 48 - DNA, 39 bp, Artificial Sequence
  Oligonucleotide primer
ccggaattcggttctcattacaacatccagtttgataaa

SEQ ID NO: 49 - DNA, 39 bp, Artificial Sequence
  Oligonucleotide primer
ccggaattcctccaagaaaagatctcatgcatcaccagg

SEQ ID NO: 50 - DNA, 39 bp, Artificial Sequence
  Oligonucleotide primer
cccaagcttaatttagttttcacggggcatctgcagggt

SEQ ID NO: 51 - DNA, 39 bp, Artificial Sequence
  Oligonucleotide primer
cccaagctttgcacaccctagggtatgttcttgcaattc

SEQ ID NO: 52 - DNA, 39 bp, Artificial Sequence
  Oligonucleotide primer
cccaagcttcccaaagccatgccctgctccagaaacacg

SEQ ID NO: 53 - DNA, 39 bp, Artificial Sequence
  Oligonucleotide primer
ccggaattcccataatatgtgaatgcgttatttagggaa

SEQ ID NO: 54 - DNA, 29 bp, Artificial Sequence
  Oligonucleotide primer
cccaagcttcatgttccacgcattacgtc

SEQ ID NO: 55 - DNA, 29 bp, Artificial Sequence
  Oligonucleotide primer
cccaagcttaagagtgtttgggttcaccg

SEQ ID NO: 56 - DNA, 39 bp, Artificial Sequence
  Oligonucleotide primer
cccaagcttttaactcaggaggactcaatacaccctgga

SEQ ID NO: 57 - DNA, 39 bp, Artificial Sequence
  Oligonucleotide primer
cccaagcttaaacaatacctacaaattcagaagctcttt

SEQ ID NO: 58 - DNA, 38 bp, Artificial Sequence
  Oligonucleotide primer
cccaagcttaagtcttctggttacaccttcaccattat

SEQ ID NO: 59 - DNA, 39 bp, Artificial Sequence
  Oligonucleotide primer
cccaagcttactctcttccctctgtgactagagctctgt

SEQ ID NO: 60 - DNA, 39 bp, Artificial Sequence
  Oligonucleotide primer
cccaagctttcagcttctacagttgtgtcacccatgtgt

SEQ ID NO: 61 - DNA, 39 bp, Artificial Sequence
  Oligonucleotide primer
cccaagctttgcagagttcactgggtttcctaaaggcaa

SEQ ID NO: 62 - DNA, 39 bp, Artificial Sequence
  Oligonucleotide primer
cccaagctttcaccacaaaggaactttcatctctcctgg

SEQ ID NO: 63 - DNA, 39 bp, Artificial Sequence
  Oligonucleotide primer
cccaagctttttcacacagaaatgtttagaggtcaggcc

SEQ ID NO: 64 - DNA, 39 bp, Artificial Sequence; identical to SEQ ID NO: 51
  Oligonucleotide primer

SEQ ID NO: 65 - DNA, 39 bp, Artificial Sequence; identical to SEQ ID NO: 52
  Oligonucleotide primer

SEQ ID NO: 66 - DNA, 33 bp, Artificial Sequence
  Oligonucleotide primer
cccaagcttatgatgtaaccctcattggcctca

SEQ ID NO: 67 - DNA, 33 bp, Artificial Sequence
  Oligonucleotide primer
ccggaattcgagactccaagaaaagatctcatg

SEQ ID NO: 68 - DNA, 33 bp, Artificial Sequence
  Oligonucleotide primer
cccaagcttatgttcttgcaattcagcggagga

SEQ ID NO: 69 - DNA, 33 bp, Artificial Sequence
  Oligonucleotide primer
ccggaattctgtgtgagatctcaggagaaggta

SEQ ID NO: 70 - DNA, 39 bp, Artificial Sequence
  Oligonucleotide primer
cccaagcttcggccccgatgcgggactgcgttttgacca

SEQ ID NO: 71 - DNA, 39 bp, Artificial Sequence
  Oligonucleotide primer
ccggaattcataacaagctaatttaaaaaactttttgaa

SEQ ID NO: 72 - DNA, 39 bp, Artificial Sequence
  Oligonucleotide primer
cccaagcttgcacagacgggaggtacggtatggacgtct

SEQ ID NO: 73 - DNA, 39 bp, Artificial Sequence
  Oligonucleotide primer
ccggaattcaaaaaaataaacttgatttatgatggtcaa

SEQ ID NO: 74 - DNA, 39 bp, Artificial Sequence
  Oligonucleotide primer
ccggaattccgcggtgacctgcttcctgccacctgctgt

SEQ ID NO: 75 - DNA, 29 bp, Artificial Sequence
  Oligonucleotide primer
ccggaattcagttagtgcagccaagccct

SEQ ID NO: 76 - DNA, 39 bp, Artificial Sequence
  Oligonucleotide primer
ccggaattcaaaaggcaagtggacttcggtgcttacctg

SEQ ID NO: 77 - DNA, 36 bp, Artificial Sequence
  Oligonucleotide primer
cccaagcttcagctcagctcagttcagttcagccct

SEQ ID NO: 78 - DNA, 39 bp, Artificial Sequence
  Oligonucleotide primer
cccaagcttatgcgagggtctggacggctgaggaccccc

SEQ ID NO: 79 - DNA, 39 bp, Artificial Sequence
  Oligonucleotide primer
ccggaattcatgcggcaagggttgcggaccgctggctgg

SEQ ID NO: 80 - DNA, 29 bp, Artificial Sequence
  Oligonucleotide primer
ccggaattcgcccagcccagcctagctca

SEQ ID NO: 81 - DNA, 29 bp, Artificial Sequence
  Oligonucleotide primer
cccaagcttttatcaactgctagtttgtg

SEQ ID NO: 82 - DNA, 36 bp, Artificial Sequence
  Oligonucleotide primer
ccggaattcagggctgaactgaactgagctgagctg

SEQ ID NO: 83 - DNA, 35 bp, Artificial Sequence
  Oligonucleotide primer
cggctgatatctgggagcctctgtggattttccga

SEQ ID NO: 84 - DNA, 31 bp, Artificial Sequence
  Oligonucleotide primer
agccggatatcgcccagcccagcctagctca

SEQ ID NO: 85 - DNA, 24 bp, Artificial Sequence
  Oligonucleotide primer
gaaagttaaatgggagtgacccag

SEQ ID NO: 86 - DNA, 24 bp, Artificial Sequence
  Oligonucleotide primer
gagtgaccatcgcacccttgacag

SEQ ID NO: 87 - DNA, 10826 bp, Artificial Sequence
  Plasmid 3kb15a-7-4T
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtctgtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgacttaaaaatgtgagagattttcaagtatattaatttttttaactgtccaagtatttgaaattcttatcatttgattaacacccatgagtgatatgtgtctggaattgaggccaaagcaagctcagctaagaaatactagcacagtgctgtcggccccgatgcgggactgcgttttgaccatcataaatcaagtttatttttttaattaattgagcgaagctggaagcagatgatgaattagagtcaagatggctgcatgggggtctccggcacccacagcaggtggcaggaagcaggtcaccgcgagagtctattttaggaagcaaaaaaacacaattggtaaatttatcacttctggttgtgaagaggtggttttgcccaggcccagatctgaaagtgctctactgagcaaaacaacacctggacaatttgcgtttctaaaataaggcgaggctgaccgaaactgaaaaggctttttttaactatctgaatttcatttccaatcttagcttatcaactgctagtttgtgcaaacagcatatcaacttctaaactgcattcatttttaaagtaagatgtttaagaaattaaacagtcttagggagagtttatgactgtattcaaaaagttttttaaattagcttgttatcccttcatgtgataattaatctcaaatactttttcgatacctcagagcattattttcataatgactgtgttcacaatctttttaggttaactcgttttctctttgtgattaaggagaaacactttgatattctgatagagtggccttcattttagtatttttcaagaccacttttcaactactcactttaggataagttttaggaaaaatgtgcatcattatcctgaattatttcagttaagcatgttagttggtggcataagagaaaactcaatcagatagtgctgaagacaggactgtggagacaccttagaaggacagattctgttccgaatcaccgatgcggcgtcagcaggactggcctagcggaggctctgggagggtggctgccaggcccggcctgggctctgggtctccccggactacccagagctgggatgcgtggcttctgctgccgggccgactggctgctcaggccccagcccttgttaatggacttggaggaatgattccatgccaaagctttgcaaggctcgcagtgaccaggcgcccgacatggtaagagacaggcagccgccgctgctgcatttgcttctcttaaaactttgtatttgacgtcttatttccactagaaggggaactggtcttaattgcttgatgaagagcaggagactcatttatgtgagtcttttgagtgaccattgtctgggtcactcccatttaactttccctaaagcccatttgaaggagaggtcgcacgagctgctccacaacctctgaatggggatggcatgggtaatgatgcttgagaacataccaagtcccactggcatcgcccttgtctaagtcattgactgtaggtcatcatcgcacccttgaaagtagcccatgccttccaaagcgatttatggtaaatggcagaattttaagtggcaaattcagataaaatgcatttcttggttgtttccaatgatgactgttatctagagggaatttaaaggcaggggtttactgcagactcagaagggaggggatgctccgggaaggtggaggctctgagcatctcaataccctcctcttggtgcagaagatatgctgccacttctagagcaaggggacctgctcatttttatcacagcacaggctcctaaattcttggtctcattctcaagatgttttaatgactttaaagcagcaaagaaatattccacccaggtagtggagggtggtaatgattggtaatgctttggaaccaaaacccaggtggcgctggggcaggactgcagggaactggggtatcaagtagagggagacaaaagatggaagccagcctggctgtgcaggaacccggcaatgagatggctttagctgagacaagcaggtctggtgggctgaccatttctggccatgacaactccatccagttttcagaaatggactcagatgggcaaaactgacctaagttgacctagactaaacaaggccgaactgggctgagctgagctgaactgggctgagttgaactgggctgagctgagctgagctgagctgggctaagttgcaccaggtgagctgagttgagctgggcttggctgcattaaggtgggctgagctgggcagggctgggctgaattgagctgggctgggctgagctaggctgggctgggcgatatcgaattcctgcagcccgggggatccgcccccatcgataatttagttttcacggggcatctgcagggttccaaagtgagaccaggtgaattcaatgtgcatgcacttcccaagtgtccacttgtattctgtttctttacttctgtttacagaaagtagacacatattcagtcttagtaccagtgtagggagcgctttccatgagatggataccagaaaaaaatggcaaacatgggatccgttaatataaaaattagccacgatgtatatatatatatgtgtgtgtgtgtgtgtgtgtgtgtgtacacacgcgcgcgcatgtgtgagttgaatagcagagttggagtgggtttctatccacatgtacctgcacctgcaggtattctcaggtgccataatcaactgtaggaccctaaaggaaataagagtctcccctcaacccctgaagagtgtttgggttcaccgtgtgtccaatgattctgtgcctcttgagctccaggaaagggctccctggtgatgcatgagatcttttcttggagtctctctgcagagttcactgggtttcctaaaggcaattcactatttcaaaagatggtgtgaggagcatgtggtgtccctaaaggagaattctgagccagggcacaaccactttatactgagctggatacactggtaggaatatactctgtcagctcagatagaaacctccctgcatggttggggctgggctgcagggggcgctccggatacacccagcacaggctcccgccccagagcaggtgcacaggaggctggggagaggttcctcccagggcctgggacttcctttaaaaatatctaaaataagtatttcacaaagactgctgaagtttgtataaatatctattcaattgtgagcatttatcaaactggatgttgtaatgagaaccacttttacaatggggatttcaaactctgctggaggtcaggaagagatcctttcttataaataaatgcaatttttggataaacacagtcattccctaaataacgcattcacatattatggtctagaaatgatgcaagttgaccctgagacagtcaaatgtggtttcaaagtgaggtgctgtccttgaggagcttgttctccagtgggggaagctctgtcaacacagagttcagggatgtgtaggggacacatggcctctaacaggattacggcttgaaccctcagcttctacagttgtgtcacccatgtgtctgtttctcatactgggtcaggaattgggctattaaatagcatccttcatgaatatgcaattaactgaggtgactatagtatctccgttccctgagagcctcacccaacaaccacacccctcctctggagaagcccctagatcacagctcctcaccatggactggacctgaaggatcctcttcttgatggcagcagcaacaggtaaggggctccccagtctcagggctgaggaagaaaccaggccagtcatgtgagacttcacccactcttgtgtccactccacaggtgcccactccctgcagctggtgcagtctgggcctgaggtgaagaagcctggggcctcagtgaaggtctcctataagtcttctggttacaccttcaccatctatggtatgaattgggtatgatagacccctggacagggctttgagtggatgtgatggatcatcacctacactgggaacccaacgtatacccacggcttcacaggatggtttgtcttctccatggacacgtctgtcagcacggcgtgtcttcagatcagcagcctaaaggctgaggacacggccgagtattactgtgcgaagtacacagtgtggaaacccacatcccgagagtttcagaaagcctgaggaaggaggcagctgtgctgagctgaggcagtggtacagcagttttctgaacttccatagtatctcattttgcattgagttccgctttaatattagccaagaatatgggatagacgggtgctcctaagagatccttaacttgcccattttgatgggttttcccaaagacgtgagaagccacttttttcgcaaagcatcccaaagccatgccctgctccagaaacacgtgtatccatttcctggtctttgattaactgacaaactctcatcagcgcacctgggctaatttcacatcaggtagaaatatgtgctttaaagcaaggctaacgttgtaatagcaattcctgcttaataaccttcagcattgttgttgtgtgctccatcaactaattacgttagttcaaggttctcaatgggagtttctaataaatataagggatgtatagaagttcccctaattaaaacaattgtgaagacaacctcagtgttcaaccatatttcaacccttcaccacaaaggaactttcatctctcctggaagttgggttcattttcaaattagtttttttattttaatatctcaagattattgtatgtgactattttagcagaaagtgaattatgggaacttgaactaaccaactgaaaatacattcagaactaattaaacaagatgccagaatgtgattggctccaggcattttaaattcaacaggttatgtaaccaggctttaaatttgcacatcttcgtgttaccttcatgacacagtcaactcccattatgtaagaaatggtgagtgcattcccaagggtcttgcacagttataaaaatagacttgatgaggtgaggagttgtttaaattcccctctgaagaagcagcatcaacccaacaaaccactctcttccctctgtgactagagctctgtcacaggccacatggacctaaatccttgatggagattacaggactacgtaaattggactgatcgtttttatgctgttaaattaataggtgagtctgcactccagcctgggcaacagaataatcttgtctgtaaaatacaaaagaaagataaattaatagatactgactttgacatttcggataataatattttcataaaccgaatttaattatacccacattgttacctacaccttcactgaaaagttcctagttatgttgagttccatcaacactccacatgttcaaatctggacatccaagagagtctagagaataaaacgcaatgagggcagtgaaacttgcgtatattcagcacctcttaactcaggaggactcaatacaccctggaacactctgcttttctgaatggctcacaatgactccagctcactctccaacctcctcaaacatctggcctctgtttgccctaagttcacgctctgctcttagtctatgttctgaagtctttgtagaggtgaaaatgagctgtcagatggatcttccttctcactgcaacatggaatttgctatttcacttaatgaccactctttccacaatggttgatttcttttggcctgttcattactggtgattttcaagggaatctcagttgaatctttactgttttgcattttgtctccatgacaatgttgggaagtttttcttctagcagcataacatgatctagtgacctgacacatttgcagcaaacaatacctacaaattcagaagctctttggttttctttccacgaaatataattcttgctcttctgtgtatgagcacatcctagcatccctgtacacacccaggtagatgtaaacacgccgatgaaatattccctgtaaataaaaaaagtatctcagtttctctcaatgttcataattctcctgagggtgaggaaggtacttctgggtctgctcaaacaaatggcccagagaccacctggtaggtaggtaaggagctcacctcgctctggatattgagtctgtctctttccctctgtcgtctcatagaaggccagcccacttgttcagctcctaagaagagagcccaggtttatccagattatacaacacaaccagcttctgatgactctcctgttacaacatccatggagatattttgtgtattatataattcaccaaactaatgtgaaatgcccaagttgcaatactgcacaccctagggtatgttcttgcaattcagcggaggagaaattctttcagagacagatggatctgaattggtaaatatgtgggtacgaattctgggtttgagtgtcattgtccagccatgtttcacaggtgtgacctgtcagggaagaaccagagttccttgttctctcagagggtagagctcacagaggtcctctctggttcccaggaaaggtaatttcactaatcttggtgatgagactatcctccagtgctgatgtactatagagttttcatctgaagctgtcactgctatccccaatgtacatcttttcacacagaaatgtttagaggtcaggccatattctcagggttacacattgagaaggatggagatatattctactaccttctcctgagatctcacacacaatctcaaatttcaaaaggtctcagaagggcagctctcaggtactatttaaaaataacccacttcctgggacaggtagcatccttctaaccatgatggatgttctgaactacagtacacattgcatggatccaggtttgtctcaattcactgtgattattacactcagcagctgtttcaatatgtctgaaggggtaaatgacaatttaggtgacctgggtgtatggttggtgttatatgaatctttaaatgtagaacagtattaactgtattccaaaatctgtctttgatccatgatcacacttgtctcccagaccagctccttcagcacatttcctacctggaagaagaggactctgggtttggtgaggggaggccacaggaagagaactgagttctcagagggcacagccagcatacacctcccagggtgagcccaaaagactggggcctccctcatccctttttacctatccatacaaaggcaccacccacatgcaaatcctcacttaggcacccacaggaaatgactacacatttccttaaattcagggtccagctcacatgggaagtgctttctgagagtcatggacctcctgcacaagaacggtaccgggctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 88 - DNA, 42 bp, Artificial Sequence
  Oligonucleotide Primer
gacggtatcgataagcttgatatcgaattcctgcagcccggg

SEQ ID NO: 89 - DNA, 42 bp, Artificial Sequence
  Oligonucleotide primer
gacctcctgcacaagaacggtaccgggctagagcggccgcca

SEQ ID NO: 90 - DNA, 25 bp, Artificial Sequence
  Oligonucleotide primer
cagtgctgcaatgataccgcgagac

SEQ ID NO: 91 - DNA, 13722 bp, Artificial Sequence
  Plasmid KW2
cagtgtggttttgcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggcaataaaaagacagaataaaacgcacgggtgttgggtcgtttgttcggatccgtcgactttatcatcatcatctttataatctttatcatcatcatctttataatcgttatgtcctaatttcgaaggcacttgggagtagctagccaccacagactcgtgctgctcaacggtcttccaatcgtcacttatgttcagcttttccatacgcaaatgatgaaagtgaagttctggaagctttacctctttcttcgacttgtaagttgtttccatgaagcaagaaagatgacctccgtcagccagcataagtgcgggggtatcgcgaagcagaagtccaccatctcgtgggatgactgtttcagttgatggctcccatccacaggtcttcttttgcatcactggaccgtttgcgggaaaattggtgccaaggactttaactttgcagatgatgcaatcaccctgcaaacttgtttcttgggtagctgaaagcactcctccatcttcataggtgcttactctttcccaagaaaaaccaccaggtagagactgcttaaagaagtcaggaatttctttggggtacttggcgaagaccttgatgccatactgaaaggcgtgtgacagaatgtcaaaagcaaatggcagaggacctccttcagttattgtaatattcaggctttgggtgccttcgtatggttttccctctccttttccagtgcacttgaaggcgtggtagttaacactaccctccatagaaagctgaacgcgcatggtttccttgatggaaggatacatggtaccatgactctcagaaagcacttcccatgtgagctggaccctgaatttaaggaaatgtgtagtcatttcctgtgggtgcctaagtgaggatttgcatgtgggtggtgcctttgtatggataggtaaaaagggatgagggaggccccagtcttttgggctcaccctgggaggtgtatgctggctgtgccctctgagaactcagttctcttcctgtggcctcccctcaccaaacccagagtcctcttcttccaggtaggaaatgtgctgaaggagctggtctgggagacaagtgtgatcatggatcaaagacagattttggaatacagttaatactgttctacatttaaagattcatataacaccaaccatacacccaggtcacctaaattgtcatttaccccttcagacatattgaaacagctgctgagtgtaataatcacagtgaattgagacaaacctggatccatgcaatgtgtactgtagttcagaacatccatcatggttagaaggatgctacctgtcccaggaagtgggttatttttaaatagtacctgagagctgcccttctgagaccttttgaaatttgagattgtgtgtgagatctcaggagaaggtagtagaatatatctccatccttctcaatgtgtaaccctgagaatatggcctgacctctaaacatttctgtgtgaaaagatgtacattggggatagcagtgacagcttcagatgaaaactctatagtacatcagcactggaggatagtctcatcaccaagattagtgaaattacctttcctgggaaccagagaggacctctgtgagctctaccctctgagagaacaaggaactctggttcttccctgacaggtcacacctgtgaaacatggctggacaatgacactcaaacccagaattcgtacccacatatttaccaattcagatccatctgtctctgaaagaatttctcctccgctgaattgcaagaacataccctagggtgtgcagtattgcaacttgggcatttcacattagtttggtgaattatataatacacaaaatatctccatggatgttgtaacaggagagtcatcagaagctggttgtgttgtataatctggataaacctgggctctcttcttaggagctgaacaagtgggctggccttctatgagacgacagagggaaagagacagactcaatatccagagcgaggtgagctccttacctacctaccaggtggtctctgggccatttgtttgagcagacccagaagtaccttcctcaccctcaggagaattatgaacattgagagaaactgagatactttttttatttacagggaatatttcatcggcgtgtttacatctacctgggtgtgtacagggatgctaggatgtgctcatacacagaagagcaagaattatatttcgtggaaagaaaaccaaagagcttctgaatttgtaggtattgtttgctgcaaatgtgtcaggtcactagatcatgttatgctgctagaagaaaaacttcccaacattgtcatggagacaaaatgcaaaacagtaaagattcaactgagattcccttgaaaatcaccagtaatgaacaggccaaaagaaatcaaccattgtggaaagagtggtcattaagtgaaatagcaaattccatgttgcagtgagaaggaagatccatctgacagctcattttcacctctacaaagacttcagaacatagactaagagcagagcgtgaacttagggcaaacagaggccagatgtttgaggaggttggagagtgagctggagtcattgtgagccattcagaaaagcagagtgttccagggtgtattgagtcctcctgagttaagaggtgctgaatatacgcaagtttcactgccctcattgcgttttattctctagactctcttggatgtccagatttgaacatgtggagtgttgatggaactcaacataactaggaacttttcagtgaaggtgtaggtaacaatgtgggtataattaaattcggtttatgaaaatattattatccgaaatgtcaaagtcagtatctattaatttatctttcttttgtattttacagacaagattattctgttgcccaggctggagtgcagactcacctattaatttaacagcataaaaacgatcagtccaatttacgtagtcctgtaatctccatcaaggatttaggtccatgtggcctgtgacagagctctagtcacagagggaagagagtggtttgttgggttgatgctgcttcttcagaggggaatttaaacaactcctcacctcatcaagtctatttttataactgtgcaagacccttgggaatgcactcaccatttcttacataatgggagttgactgtgtcatgaaggtaacacgaagatgtgcaaatttaaagcctggttacataacctgttgaatttaaaatgcctggagccaatcacattctggcatcttgtttaattagttctgaatgtattttcagttggttagttcaagttcccataattcactttctgctaaaatagtcacatacaataatcttgagatattaaaataaaaaaactaatttgaaaatgaacccaacttccaggagagatgaaagttcctttgtggtgaagggttgaaatatggttgaacactgaggttgtcttcacaattgttttaattaggggaacttctatacatcccttatatttattagaaactcccattgagaaccttgaactaacgtaattagttgatggagcacacaacaacaatgctgaaggttattaagcaggaattgctattacaacgttagccttgctttaaagcacatatttctacctgatgtgaaattagcccaggtgcgctgatgagagtttgtcagttaatcaaagaccaggaaatggatacacgtgtttctggagcagggcatggctttgggatgctttgcgaaaaaagtggcttctcacgtctttgggaaaacccatcaaaatgggcaagttaaggatctcttaggagcacccgtctatcccatattcttggctaatattaaagcggaactcaatgcaaaatgagatactatggaagttcagaaaactgctgtaccactgcctcagctcagcacagctgcctccttcctcaggctttctgaaactctcgggatgtgggtttccacactgtgtacttcgcacagtaatactcggccgtgtcctcagcctttaggctgctgatctgaagacacgccgtgctgacagacgtgtccatggagaagacaaaccatcctgtgaagccgtgggtatacgttgggttcccagtgtaggtgatgatccatcacatccactcaaagccctgtccaggggtctatcatacccaattcataccatagatggtgaaggtgtaaccagaagacttataggagaccttcactgaggccccaggcttcttcacctcaggcccagactgcaccagctgcagggagtgggcacctgtggagtggacacaagagtgggtgaagtctcacatgactggcctggtttcttcctcagccctgagactggggagccccttacctgttgctgctgccatcaagaagaggatccttcaggtccagtccatggtgaggagctgtgatctaggggcttctccagaggaggggtgtggttgttgggtgaggctctcagggaacggagatactatagtcacctcagttaattgcatattcatgaaggatgctatttaatagcccaattcctgacccagtatgagaaacagacacatgggtgacacaactgtagaagctgagggttcaagccgtaatcctgttagaggccatgtgtcccctacacatccctgaactctgtgttgacagagcttcccccactggagaacaagctcctcaaggacagcacctcactttgaaaccacatttgactgtctcagggtcaacttgcatcatttctagaccataatatgtgaatgcgttatttagggaatgactgtgtttatccaaaaattgcatttatttataagaaaggatctcttcctgacctccagcagagtttgaaatccccattgtaaaagtggttctcattacaacatccagtttgataaatgctcacaattgaatagatatttatacaaacttcagcagtctttgtgaaatacttattttagatatttttaaaggaagtcccaggccctgggaggaacctctccccagcctcctgtgcacctgctctggggcgggagcctgtgctgggtgtatccggagcgccccctgcagcccagccccaaccatgcagggaggtttctatctgagctgacagagtatattcctaccagtgtatccagctcagtataaagtggttgtgccctggctcagaattctcctttagggacaccacatgctcctcacaccatcttttgaaatagtgaattgcctttaggaaacccagtgaactctgcagagagactccaagaaaagatctcatgcatcaccagggagccctttcctggagctcaagaggcacagaatcattggacacacggtgaacccaaacactcttcaggggttgaggggagactcttatttcctttagggtcctacagttgattatggcacctgagaatacctgcaggtgcaggtacatgtggatagaaacccactccaactctgctattcaactcacacatgcgcgcgcgtgtgtacacacacacacacacacacacacacatatatatatatacatcgtggctaatttttatattaacggatcccatgtttgccatttttttctggtatccatctcatggaaagcgctccctacactggtactaagactgaatatgtgtctactttctgtaaacagaagtaaagaaacagaatacaagtggacacttgggaagtgcatgcacattgaattcacctggtctcactttggaaccctgcagatgccccgtgaaaactaaattatcgattgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggcaaacgtgcgcgccaggtcgcagatcgtcggtatggagcctggggtggtgacgtgggtctggaccatcccggaggtaagttgcagcagggcgtcccggtagccggcgggcgattggtcgtaatccaggataaagacgtgcatgggacggaggcgtttggccaagacgtccaaggcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgtccccgaaagctgtccccaatcctcccgccacgacccgccgccctgcagataccgcaccgtattggcaagcagcccgtaaacgcggcgaatcgcggccagcatagccaggtcaagccgctcgccggggcgctggcgtttggccaggcggtcgatgtgtctgtcctccggaagggcccccaacacgatgtttgtgccgggcaaggtcggcgggatgagggccacgaacgccagcacggcctggggggtcatgctgcccataaggtatcgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacctgccagtaagtcatcggctcgggtacgtagacgatatcgtcgcgcgaacccagggccaccagcagttgcgtggtggtggttttccccatcccgtgaggaccgtctatataaacccgcagtagcgtgggcattttctgctccaggcggacttccgtggcttcttgctgccggcgagggcgcaacgccgtacgtcggttgctatggccgcgagaacgcgcagcctggtcgaacgcagacgcgtgttgatggcaggggtacgaagccatacgcgcttctacaaggcgcttgccgaagaggtgcgggagtttcacgccaccaagatctgcggcacgctgttgacgctgttaagcgggtcgctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgcaagcttggcgtaatcatggtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgcccagcccagcctagctcagcccagcccagctcaattcagcccagccctgcccagctcagcccaccttaatgcagccaagcccagctcaactcagctcacctggtgcaacttagcccagctcagctcagctcagctcagcccagttcaactcagcccagttcagctcagctcagcccagttcggccttgtttagtctaggtcaacttaggtcagttttgcccatctgagtccatttctgaaaactggatggagttgtcatggccagaaatggtcagcccaccagacctgcttgtctcagctaaagccatctcattgccgggttcctgcacagccaggctggcttccatcttttgtctccctctacttgataccccagttccctgcagtcctgccccagcgccacctgggttttggttccaaagcattaccaatcattaccaccctccactacctgggtggaatatttctttgctgctttaaagtcattaaaacatcttgagaatgagaccaagaatttaggagcctgtgctgtgataaaaatgagcaggtccccttgctctagaagtggcagcatatcttctgcaccaagaggagggtattgagatgctcagagcctccaccttcccggagcatcccctcccttctgagtctgcagtaaacccctgcctttaaattccctctagataacagtcatcattggaaacaaccaagaaatgcattttatctgaatttgccacttaaaattctgccatttaccataaatcgctttggaaggcatgggctactttcaagggtgcgatgatgacctacagtcaatgacttagacaagggcgatgccagtgggacttggtatgttctcaagcatcattacccatgccatccccattcagaggttgtggagcagctcgtgcgacctctccttcaaatgggctttagggaaagttaaatgggagtgacccagacaatggtcactcaaaagactcacataaatgagtctcctgctcttcatcaagcaattaagaccagttccccttctagtggaaataagacgtcaaatacaaagttttaagagaagcaaatgcagcagcggcggctgcctgtctcttaccatgtcgggcgcctggtcactgcgagccttgcaaagctttggcatggaatcattcctccaagtccattaacaagggctggggcctgagcagccagtcggcccggcagcagaagccacgcatcccagctctgggtagtccggggagacccagagcccaggccgggcctggcagccaccctcccagagcctccgctaggccagtcctgctgacgccgcatcggtgattcggaacagaatctgtccttctaaggtgtctccacagtcctgtcttcagcactatctgattgagttttctcttatgccaccaactaacatgcttaactgaaataattcaggataatgatgcacatttttcctaaaacttatcctaaagtgagtagttgaaaagtggtcttgaaaaatactaaaatgaaggccactctatcagaatatcaaagtgtttctccttaatcacaaagagaaaacgagttaacctaaaaagattgtgaacacagtcattatgaaaataatgctctgaggtatcgaaaaagtatttgagattaattatcacatgaagggataacaagctaatttaaaaaactttttgaatacagtcataaactctccctaagactgtttaatttcttaaacatcttactttaaaaatgaatgcagtttagaagttgatatgctgtttgcacaaactagcagttgataagctaagattggaaatgaaattcagatagttaaaaaaagccttttcagtttcggtcagcctcgccttattttagaaacgcaaattgtccaggtgttgttttgctcagtagagcactttcagatctgggcctgggcaaaaccacctcttcacaaccagaagtgataaatttaccaattgtgtttttttgcttcctaaaatagactctcgcggtgacctgcttcctgccacctgctgtgggtgccggagacccccatgcagccatcttgactctaattcatcatctgcttccagcttcgctcaattaattaaaaaaataaacttgatttatgatggtcaaaacgcagtcccgcatcggggccgacagcactgtgctagtatttcttagctgagcttgctttggcctcaattccagacacatatcactcatgggtgttaatcaaatgataagaatttcaaatacttggacagttaaaaaaattaatatacttgaaaatctctcacatttttaagtcataattttcttaaccatttttctcagaagccacttcaaacatatcctgtcttttaacagtaagcatgcctcctaagataaacaatccttttctcatggaaaccagcttcaaggcactgaggtcctggagcctccctaagcccctgtcaggacggcagccaccgtttctgggctacccctgcccccaaccctgctctcatcaagaccggggctacgcgtccctcctggctggattcacccactccgacagttctctttccagccaataaagaatttaagatgcaggttgacacacagcgcacctcataattctaaagaaaatatttcacgattcgctgctgtgcagcgatcttgcagtcctacagacaccgctcctgagacacattcctcagccatcactaagacccctggtttgttcaggcatctcgtccaaatgtggctccccaagcccccaggctcagttactccatcagacgcacccaacctgagtcccattttccaaaggcatcggaaaatccacagaggctcccactcgag

SEQ ID NO: 92 - DNA, 33 bp, Artificial Sequence
  Oligonucleotide primer
ccgctcgagatgtatccttccatcaaggaaacc

SEQ ID NO: 93 - DNA, 69 bp, Artificial Sequence
  Oligonucleotide primer
tctagattattatttatcatcatcatctttataatctttatcatcatcatctttataatcagcggccgc

SEQ ID NO: 94 - DNA, 31 bp, Artificial Sequence
  Oligonucleotide primer
ctagtctagattattatttatcatcatcatc

SEQ ID NO: 95 - DNA, 33 bp, Artificial Sequence
  Oligonucleotide primer
gttatgtcctaatttcgaaggcacttgggagta

SEQ ID NO: 96 - protein, 30 aa, Homo sapiens
MKTPWKVLLGLLGAAALVTIITVPVVLLNK

SEQ ID NO: 97 - DNA, 90 bp, Homo sapiens
atgaagacaccgtggaaggttctcctgggactgctgggtgctgctgcgcttgtcaccatcatcaccgtgcccgtggttctgctgaacaaa

SEQ ID NO: 98 - DNA, 50 bp, Artificial Sequence
  Oligonucleotide primer
cgtgcccgtggttctgctgaacaaaatgtatccttccatcaaggaaacca

SEQ ID NO: 99 - DNA, 51 bp, Artificial Sequence
  Oligonucleotide primer
ccgtgcccgtggttctgctgaacaaagtgtatccttccatcaaggaaacca

SEQ ID NO: 100 - DNA, 52 bp, Artificial Sequence
  Oligonucleotide primer
tgctgctgcgcttgtcaccatcatcaccgtgcccgtggttctgctgaacaaa

SEQ ID NO: 101 - DNA, 51 bp, Artificial Sequence
  Oligonucleotide primer
ggaaggttctcctgggactgctgggtgctgctgcgcttgtcaccatcatca

SEQ ID NO: 102 - DNA, 47 bp, Artificial Sequence
  Oligonucleotide primer
ccggaattcatgaagacaccgtggaaggttctcctgggactgctggg

SEQ ID NO: 103 - DNA, 49 bp, Artificial Sequence
  Oligonucleotide primer
gactagtttattatttatcatcatcatctttataatctttatcatcatc

SEQ ID NO: 104 - DNA, 3735 bp, Artificial Sequence
  Plasmid pME18sCD26asfp499
aagcttggctgtggaatgtgtgtcagttagggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgatcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctctgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattcatgaagacaccgtggaaggttctcctgggactgctgggtgctgctgcgcttgtcaccatcatcaccgtgcccgtggttctgctgaacaaaatgtatccttccatcaaggaaaccatgcgcgttcagctttctatggagggtagtgttaactaccacgccttcaagtgcactggaaaaggagagggaaaaccatacgaaggcacccaaagcctgaatattacaataactgaaggaggtcctctgccatttgcttttgacattctgtcacacgcctttcagtatggcatcaaggtcttcgccaagtaccccaaagaaattcctgacttctttaagcagtctctacctggtggtttttcttgggaaagagtaagcacctatgaagatggaggagtgctttcagctacccaagaaacaagtttgcagggtgattgcatcatctgcaaagttaaagtccttggcaccaattttcccgcaaacggtccagtgatgcaaaagaagacctgtggatgggagccatcaactgaaacagtcatcccacgagatggtggacttctgcttcgcgatacccccgcacttatgctggctgacggaggtcatctttcttgcttcatggaaacaacttacaagtcgaagaaagaggtaaagcttccagaacttcactttcatcatttgcgtatggaaaagctgaacataagtgacgattggaagaccgttgagcagcacgagtctgtggtggctagctactcccaagtgccttcgaaattaggacataacgcggccgctgattataaagatgatgatgataaagattataaagatgatgatgataaataataaactagtctagagaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccgggtaccgagctcgaattaattcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgc

SEQ ID NO: 105 - DNA, 28 bp, Artificial Sequence
  Oligonucleotide primer
atggacagcctcttgatgaaccggagga

SEQ ID NO: 106 - DNA, 25 bp, Artificial Sequence
  Oligonucleotide primer
caaagtcccaaagtacgaaatgcgt

SEQ ID NO: 107 - DNA, 596 bp, Homo sapiens
atggacagcctcttgatgaaccggaggaagtttctttaccaattcaaaaatgtccgctgggctaagggtcggcgtgagacctacctgtgctacgtagtgaagaggcgtgacagtgctacatccttttcactggactttggttatcttcgcaataagaacggctgccacgtggaattgctcttcctccgctacatctcggactgggacctagaccctggccgctgctaccgcgtcacctggttcacctcctggagcccctgctacgactgtgcccgacatgtggccgactttctgcgagggaaccccaacctcagtctgaggatcttcaccgcgcgcctctacttctgtgaggaccgcaaggctgagcccgaggggctgcggcggctgcaccgcgccggggtgcaaatagccatcatgaccttcaaagattatttttactgctggaatacttttgtagaaaaccatgaaagaactttcaaagcctgggaagggctgcatgaaaattcagttcgtctctccagacagcttcggcgcatccttttgcccctgtatgaggttgatgacttacgagacgcatttcgtactttgggactttg

SEQ ID NO: 108 - DNA, 33 bp, Artificial Sequence
  Oligonucleotide primer
ccctcgagatggacagcctcttgatgaaccgga

SEQ ID NO: 109 - DNA, 33 bp, Artificial Sequence
  Oligonucleotide primer
gctctagacaaagtcccaaagtacgaaatgcgt

SEQ ID NO: 110 - DNA, 12990 bp, Artificial Sequence
  Plasmid KW3
gtcgacggtaccatgactctcagaaagcacttcccatgtgagctggaccctgaatttaaggaaatgtgtagtcatttcctgtgggtgcctaagtgaggatttgcatgtgggtggtgcctttgtatggataggtaaaaagggatgagggaggccccagtcttttgggctcaccctgggaggtgtatgctggctgtgccctctgagaactcagttctcttcctgtggcctcccctcaccaaacccagagtcctcttcttccaggtaggaaatgtgctgaaggagctggtctgggagacaagtgtgatcatggatcaaagacagattttggaatacagttaatactgttctacatttaaagattcatataacaccaaccatacacccaggtcacctaaattgtcatttaccccttcagacatattgaaacagctgctgagtgtaataatcacagtgaattgagacaaacctggatccatgcaatgtgtactgtagttcagaacatccatcatggttagaaggatgctacctgtcccaggaagtgggttatttttaaatagtacctgagagctgcccttctgagaccttttgaaatttgagattgtgtgtgagatctcaggagaaggtagtagaatatatctccatccttctcaatgtgtaaccctgagaatatggcctgacctctaaacatttctgtgtgaaaagatgtacattggggatagcagtgacagcttcagatgaaaactctatagtacatcagcactggaggatagtctcatcaccaagattagtgaaattacctttcctgggaaccagagaggacctctgtgagctctaccctctgagagaacaaggaactctggttcttccctgacaggtcacacctgtgaaacatggctggacaatgacactcaaacccagaattcgtacccacatatttaccaattcagatccatctgtctctgaaagaatttctcctccgctgaattgcaagaacataccctagggtgtgcagtattgcaacttgggcatttcacattagtttggtgaattatataatacacaaaatatctccatggatgttgtaacaggagagtcatcagaagctggttgtgttgtataatctggataaacctgggctctcttcttaggagctgaacaagtgggctggccttctatgagacgacagagggaaagagacagactcaatatccagagcgaggtgagctccttacctacctaccaggtggtctctgggccatttgtttgagcagacccagaagtaccttcctcaccctcaggagaattatgaacattgagagaaactgagatactttttttatttacagggaatatttcatcggcgtgtttacatctacctgggtgtgtacagggatgctaggatgtgctcatacacagaagagcaagaattatatttcgtggaaagaaaaccaaagagcttctgaatttgtaggtattgtttgctgcaaatgtgtcaggtcactagatcatgttatgctgctagaagaaaaacttcccaacattgtcatggagacaaaatgcaaaacagtaaagattcaactgagattcccttgaaaatcaccagtaatgaacaggccaaaagaaatcaaccattgtggaaagagtggtcattaagtgaaatagcaaattccatgttgcagtgagaaggaagatccatctgacagctcattttcacctctacaaagacttcagaacatagactaagagcagagcgtgaacttagggcaaacagaggccagatgtttgaggaggttggagagtgagctggagtcattgtgagccattcagaaaagcagagtgttccagggtgtattgagtcctcctgagttaagaggtgctgaatatacgcaagtttcactgccctcattgcgttttattctctagactctcttggatgtccagatttgaacatgtggagtgttgatggaactcaacataactaggaacttttcagtgaaggtgtaggtaacaatgtgggtataattaaattcggtttatgaaaatattattatccgaaatgtcaaagtcagtatctattaatttatctttcttttgtattttacagacaagattattctgttgcccaggctggagtgcagactcacctattaatttaacagcataaaaacgatcagtccaatttacgtagtcctgtaatctccatcaaggatttaggtccatgtggcctgtgacagagctctagtcacagagggaagagagtggtttgttgggttgatgctgcttcttcagaggggaatttaaacaactcctcacctcatcaagtctatttttataactgtgcaagacccttgggaatgcactcaccatttcttacataatgggagttgactgtgtcatgaaggtaacacgaagatgtgcaaatttaaagcctggttacataacctgttgaatttaaaatgcctggagccaatcacattctggcatcttgtttaattagttctgaatgtattttcagttggttagttcaagttcccataattcactttctgctaaaatagtcacatacaataatcttgagatattaaaataaaaaaactaatttgaaaatgaacccaacttccaggagagatgaaagttcctttgtggtgaagggttgaaatatggttgaacactgaggttgtcttcacaattgttttaattaggggaacttctatacatcccttatatttattagaaactcccattgagaaccttgaactaacgtaattagttgatggagcacacaacaacaatgctgaaggttattaagcaggaattgctattacaacgttagccttgctttaaagcacatatttctacctgatgtgaaattagcccaggtgcgctgatgagagtttgtcagttaatcaaagaccaggaaatggatacacgtgtttctggagcagggcatggctttgggatgctttgcgaaaaaagtggcttctcacgtctttgggaaaacccatcaaaatgggcaagttaaggatctcttaggagcacccgtctatcccatattcttggctaatattaaagcggaactcaatgcaaaatgagatactatggaagttcagaaaactgctgtaccactgcctcagctcagcacagctgcctccttcctcaggctttctgaaactctcgggatgtgggtttccacactgtgtacttcgcacagtaatactcggccgtgtcctcagcctttaggctgctgatctgaagacacgccgtgctgacagacgtgtccatggagaagacaaaccatcctgtgaagccgtgggtatacgttgggttcccagtgtaggtgatgatccatcacatccactcaaagccctgtccaggggtctatcatacccaattcataccatagatggtgaaggtgtaaccagaagacttataggagaccttcactgaggccccaggcttcttcacctcaggcccagactgcaccagctgcagggagtgggcacctgtggagtggacacaagagtgggtgaagtctcacatgactggcctggtttcttcctcagccctgagactggggagccccttacctgttgctgctgccatcaagaagaggatccttcaggtccagtccatggtgaggagctgtgatctaggggcttctccagaggaggggtgtggttgttgggtgaggctctcagggaacggagatactatagtcacctcagttaattgcatattcatgaaggatgctatttaatagcccaattcctgacccagtatgagaaacagacacatgggtgacacaactgtagaagctgagggttcaagccgtaatcctgttagaggccatgtgtcccctacacatccctgaactctgtgttgacagagcttcccccactggagaacaagctcctcaaggacagcacctcactttgaaaccacatttgactgtctcagggtcaacttgcatcatttctagaccataatatgtgaatgcgttatttagggaatgactgtgtttatccaaaaattgcatttatttataagaaaggatctcttcctgacctccagcagagtttgaaatccccattgtaaaagtggttctcattacaacatccagtttgataaatgctcacaattgaatagatatttatacaaacttcagcagtctttgtgaaatacttattttagatatttttaaaggaagtcccaggccctgggaggaacctctccccagcctcctgtgcacctgctctggggcgggagcctgtgctgggtgtatccggagcgccccctgcagcccagccccaaccatgcagggaggtttctatctgagctgacagagtatattcctaccagtgtatccagctcagtataaagtggttgtgccctggctcagaattctcctttagggacaccacatgctcctcacaccatcttttgaaatagtgaattgcctttaggaaacccagtgaactctgcagagagactccaagaaaagatctcatgcatcaccagggagccctttcctggagctcaagaggcacagaatcattggacacacggtgaacccaaacactcttcaggggttgaggggagactcttatttcctttagggtcctacagttgattatggcacctgagaatacctgcaggtgcaggtacatgtggatagaaacccactccaactctgctattcaactcacacatgcgcgcgcgtgtgtacacacacacacacacacacacacacatatatatatatacatcgtggctaatttttatattaacggatcccatgtttgccatttttttctggtatccatctcatggaaagcgctccctacactggtactaagactgaatatgtgtctactttctgtaaacagaagtaaagaaacagaatacaagtggacacttgggaagtgcatgcacattgaattcacctggtctcactttggaaccctgcagatgccccgtgaaaactaaattatcgattgccagccctgggaccgaaccccgcgtttatgaacaaacgacccaacacccgtgcgttttattctgtctttttattgccgtcatagcgcgggttccttccggtattgtctccttccgtgtttcagttagcctcccccatctcccgggcaaacgtgcgcgccaggtcgcagatcgtcggtatggagcctggggtggtgacgtgggtctggaccatcccggaggtaagttgcagcagggcgtcccggtagccggcgggcgattggtcgtaatccaggataaagacgtgcatgggacggaggcgtttggccaagacgtccaaggcccaggcaaacacgttatacaggtcgccgttgggggccagcaactcgggggcccgaaacagggtaaataacgtgtccccgatatggggtcgtgggcccgcgttgctctggggctcggcaccctggggcggcacggccgtccccgaaagctgtccccaatcctcccgccacgacccgccgccctgcagataccgcaccgtattggcaagcagcccgtaaacgcggcgaatcgcggccagcatagccaggtcaagccgctcgccggggcgctggcgtttggccaggcggtcgatgtgtctgtcctccggaagggcccccaacacgatgtttgtgccgggcaaggtcggcgggatgagggccacgaacgccagcacggcctggggggtcatgctgcccataaggtatcgcgcggccgggtagcacaggagggcggcgatgggatggcggtcgaagatgagggtgagggccgggggcggggcatgtgagctcccagcctcccccccgatatgaggagccagaacggcgtcggtcacggcataaggcatgcccattgttatctgggcgcttgtcattaccaccgccgcgtccccggccgatatctcaccctggtcgaggcggtgttgtgtggtgtagatgttcgcgattgtctcggaagcccccagcacctgccagtaagtcatcggctcgggtacgtagacgatatcgtcgcgcgaacccagggccaccagcagttgcgtggtggtggttttccccatcccgtgaggaccgtctatataaacccgcagtagcgtgggcattttctgctccaggcggacttccgtggcttcttgctgccggcgagggcgcaacgccgtacgtcggttgctatggccgcgagaacgcgcagcctggtcgaacgcagacgcgtgttgatggcaggggtacgaagccatacgcgcttctacaaggcgcttgccgaagaggtgcgggagtttcacgccaccaagatctgcggcacgctgttgacgctgttaagcgggtcgctgcaggtcgaaaggcccggagatgaggaagaggagaacagcgcggcagacgtgcgcttttgaagcgtgcagaatgccgggcctccggaggaccttcgggcgcccgccccgcccctgagcccgcccctgagcccgcccccggacccaccccttcccagcctctgagcccagaaagcgaaggagcaaagctgctattggccgctgccccaaaggcctacccgcttccattgctcagcggtgctgtccatctgcacgagactagtgagacgtgctacttccatttgtcacgtcctgcacgacgcgagctgcggggcgggggggaacttcctgactaggggaggagtagaaggtggcgcgaaggggccaccaaagaacggagccggttggcgcctaccggtggatgtggaatgtgtgcgaggccagaggccacttgtgtagcgccaagtgcccagcggggctgctaaagcgcatgctccagactgcaagcttggcgtaatcatggtcatagctgtttcctgtgtgaaattgttatccgctcacaattccacacaacatacgagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgcccagcccagcctagctcagcccagcccagctcaattcagcccagccctgcccagctcagcccaccttaatgcagccaagcccagctcaactcagctcacctggtgcaacttagcccagctcagctcagctcagctcagcccagttcaactcagcccagttcagctcagctcagcccagttcggccttgtttagtctaggtcaacttaggtcagttttgcccatctgagtccatttctgaaaactggatggagttgtcatggccagaaatggtcagcccaccagacctgcttgtctcagctaaagccatctcattgccgggttcctgcacagccaggctggcttccatcttttgtctccctctacttgataccccagttccctgcagtcctgccccagcgccacctgggttttggttccaaagcattaccaatcattaccaccctccactacctgggtggaatatttctttgctgctttaaagtcattaaaacatcttgagaatgagaccaagaatttaggagcctgtgctgtgataaaaatgagcaggtccccttgctctagaagtggcagcatatcttctgcaccaagaggagggtattgagatgctcagagcctccaccttcccggagcatcccctcccttctgagtctgcagtaaacccctgcctttaaattccctctagataacagtcatcattggaaacaaccaagaaatgcattttatctgaatttgccacttaaaattctgccatttaccataaatcgctttggaaggcatgggctactttcaagggtgcgatgatgacctacagtcaatgacttagacaagggcgatgccagtgggacttggtatgttctcaagcatcattacccatgccatccccattcagaggttgtggagcagctcgtgcgacctctccttcaaatgggctttagggaaagttaaatgggagtgacccagacaatggtcactcaaaagactcacataaatgagtctcctgctcttcatcaagcaattaagaccagttccccttctagtggaaataagacgtcaaatacaaagttttaagagaagcaaatgcagcagcggcggctgcctgtctcttaccatgtcgggcgcctggtcactgcgagccttgcaaagctttggcatggaatcattcctccaagtccattaacaagggctggggcctgagcagccagtcggcccggcagcagaagccacgcatcccagctctgggtagtccggggagacccagagcccaggccgggcctggcagccaccctcccagagcctccgctaggccagtcctgctgacgccgcatcggtgattcggaacagaatctgtccttctaaggtgtctccacagtcctgtcttcagcactatctgattgagttttctcttatgccaccaactaacatgcttaactgaaataattcaggataatgatgcacatttttcctaaaacttatcctaaagtgagtagttgaaaagtggtcttgaaaaatactaaaatgaaggccactctatcagaatatcaaagtgtttctccttaatcacaaagagaaaacgagttaacctaaaaagattgtgaacacagtcattatgaaaataatgctctgaggtatcgaaaaagtatttgagattaattatcacatgaagggataacaagctaatttaaaaaactttttgaatacagtcataaactctccctaagactgtttaatttcttaaacatcttactttaaaaatgaatgcagtttagaagttgatatgctgtttgcacaaactagcagttgataagctaagattggaaatgaaattcagatagttaaaaaaagccttttcagtttcggtcagcctcgccttattttagaaacgcaaattgtccaggtgttgttttgctcagtagagcactttcagatctgggcctgggcaaaaccacctcttcacaaccagaagtgataaatttaccaattgtgtttttttgcttcctaaaatagactctcgcggtgacctgcttcctgccacctgctgtgggtgccggagacccccatgcagccatcttgactctaattcatcatctgcttccagcttcgctcaattaattaaaaaaataaacttgatttatgatggtcaaaacgcagtcccgcatcggggccgacagcactgtgctagtatttcttagctgagcttgctttggcctcaattccagacacatatcactcatgggtgttaatcaaatgataagaatttcaaatacttggacagttaaaaaaattaatatacttgaaaatctctcacatttttaagtcataattttcttaaccatttttctcagaagccacttcaaacatatcctgtcttttaacagtaagcatgcctcctaagataaacaatccttttctcatggaaaccagcttcaaggcactgaggtcctggagcctccctaagcccctgtcaggacggcagccaccgtttctgggctacccctgcccccaaccctgctctcatcaagaccggggctacgcgtccctcctggctggattcacccactccgacagttctctttccagccaataaagaatttaagatgcaggttgacacacagcgcacctcataattctaaagaaaatatttcacgattcgctgctgtgcagcgatcttgcagtcctacagacaccgctcctgagacacattcctcagccatcactaagacccctggtttgttcaggcatctcgtccaaatgtggctccccaagcccccaggctcagttactccatcagacgcacccaacctgagtcccattttccaaaggcatcggaaaatccacagaggctcccactcgagcagtgtggttttgcaagaggaagcaaaaagcctctccacccaggcctggaatgtttccacccaatgtcgagcagtgtggttttgcaagaggaagcaaaaagcctctccacccaggcctggaatgtttccacccaatgtcgagcaaaccccgcccagcgtcttgtcattggcgaattcgaacacgcagatgcagtcggggcggcgcggtcccaggtccacttcgcatattaaggtgacgcgtgtggcctcgaacaccgagcgaccctgcagccaatatgggatcggccattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaggggatcggcaataaaaagacagaataaaacgcacgggtgttgggtcgtttgttcggatcc

SEQ ID NO: 111 - DNA, 33 bp, Artificial Sequence
  Oligonucleotide primer
ccgctcgagtgggagcctctgtggattttccga

SEQ ID NO: 112 - DNA, 32 bp, Artificial Sequence
  Oligonucleotide primer
tgaccggacgtcgcccagcccagcctagctca

SEQ ID NO: 113 - DNA, 48 bp, Artificial Sequence
  Oligonucleotide primer
cccaagcttatggactggacctggaggatcctcttcttggtggcagca

SEQ ID NO: 114 - DNA, 51 bp, Artificial Sequence
  Oligonucleotide primer
cccaagcttatggacacactttgctccacgctcctgctgctgaccatccct

SEQ ID NO: 115 - DNA, 48 bp, Artificial Sequence
  Oligonucleotide primer
cccaagcttatggagtttgggctgagctgggttttccttgttgctatt

SEQ ID NO: 116 - DNA, 48 bp, Artificial Sequence
  Oligonucleotide primer
cccaagcttatgaaacacctgtggttcttcctcctgctggtggcagct

SEQ ID NO: 117 - DNA, 51 bp, Artificial Sequence
  Oligonucleotide primer
cccaagcttatggggtcaaccgccatcctcgccctcctcctggctgttctc

SEQ ID NO: 118 - DNA, 51 bp, Artificial Sequence
  Oligonucleotide primer
cccaagcttatgtctgtctccttcctcatcttcctgcccgtgctgggcctc

SEQ ID NO: 119 - DNA, 51 bp, Artificial Sequence
  Oligonucleotide primer
cccaagcttatggactggacctggaggatcctcttcttggtggcagcagca

SEQ ID NO: 120 - DNA, 33 bp, Artificial Sequence
  Oligonucleotide primer
ccggaattcgcggtacctgaggagacggtgacc

SEQ ID NO: 121 - DNA, 10143 bp, Artificial Sequence
  Plasmid 3kb15a7-4-TdeltaMluI-NaeI
ctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagcccgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgacttaaaaatgtgagagattttcaagtatattaatttttttaactgtccaagtatttgaaattcttatcatttgattaacacccatgagtgatatgtgtctggaattgaggccaaagcaagctcagctaagaaatactagcacagtgctgtcggccccgatgcgggactgcgttttgaccatcataaatcaagtttatttttttaattaattgagcgaagctggaagcagatgatgaattagagtcaagatggctgcatgggggtctccggcacccacagcaggtggcaggaagcaggtcaccgcgagagtctattttaggaagcaaaaaaacacaattggtaaatttatcacttctggttgtgaagaggtggttttgcccaggcccagatctgaaagtgctctactgagcaaaacaacacctggacaatttgcgtttctaaaataaggcgaggctgaccgaaactgaaaaggctttttttaactatctgaatttcatttccaatcttagcttatcaactgctagtttgtgcaaacagcatatcaacttctaaactgcattcatttttaaagtaagatgtttaagaaattaaacagtcttagggagagtttatgactgtattcaaaaagttttttaaattagcttgttatcccttcatgtgataattaatctcaaatactttttcgatacctcagagcattattttcataatgactgtgttcacaatctttttaggttaactcgttttctctttgtgattaaggagaaacactttgatattctgatagagtggccttcattttagtatttttcaagaccacttttcaactactcactttaggataagttttaggaaaaatgtgcatcattatcctgaattatttcagttaagcatgttagttggtggcataagagaaaactcaatcagatagtgctgaagacaggactgtggagacaccttagaaggacagattctgttccgaatcaccgatgcggcgtcagcaggactggcctagcggaggctctgggagggtggctgccaggcccggcctgggctctgggtctccccggactacccagagctgggatgcgtggcttctgctgccgggccgactggctgctcaggccccagcccttgttaatggacttggaggaatgattccatgccaaagctttgcaaggctcgcagtgaccaggcgcccgacatggtaagagacaggcagccgccgctgctgcatttgcttctcttaaaactttgtatttgacgtcttatttccactagaaggggaactggtcttaattgcttgatgaagagcaggagactcatttatgtgagtcttttgagtgaccattgtctgggtcactcccatttaactttccctaaagcccatttgaaggagaggtcgcacgagctgctccacaacctctgaatggggatggcatgggtaatgatgcttgagaacataccaagtcccactggcatcgcccttgtctaagtcattgactgtaggtcatcatcgcacccttgaaagtagcccatgccttccaaagcgatttatggtaaatggcagaattttaagtggcaaattcagataaaatgcatttcttggttgtttccaatgatgactgttatctagagggaatttaaaggcaggggtttactgcagactcagaagggaggggatgctccgggaaggtggaggctctgagcatctcaataccctcctcttggtgcagaagatatgctgccacttctagagcaaggggacctgctcatttttatcacagcacaggctcctaaattcttggtctcattctcaagatgttttaatgactttaaagcagcaaagaaatattccacccaggtagtggagggtggtaatgattggtaatgctttggaaccaaaacccaggtggcgctggggcaggactgcagggaactggggtatcaagtagagggagacaaaagatggaagccagcctggctgtgcaggaacccggcaatgagatggctttagctgagacaagcaggtctggtgggctgaccatttctggccatgacaactccatccagttttcagaaatggactcagatgggcaaaactgacctaagttgacctagactaaacaaggccgaactgggctgagctgagctgaactgggctgagttgaactgggctgagctgagctgagctgagctgggctaagttgcaccaggtgagctgagttgagctgggcttggctgcattaaggtgggctgagctgggcagggctgggctgaattgagctgggctgggctgagctaggctgggctgggcgatatcgaattcctgcagcccgggggatccgcccccatcgataatttagttttcacggggcatctgcagggttccaaagtgagaccaggtgaattcaatgtgcatgcacttcccaagtgtccacttgtattctgtttctttacttctgtttacagaaagtagacacatattcagtcttagtaccagtgtagggagcgctttccatgagatggataccagaaaaaaatggcaaacatgggatccgttaatataaaaattagccacgatgtatatatatatatgtgtgtgtgtgtgtgtgtgtgtgtgtacacacgcgcgcgcatgtgtgagttgaatagcagagttggagtgggtttctatccacatgtacctgcacctgcaggtattctcaggtgccataatcaactgtaggaccctaaaggaaataagagtctcccctcaacccctgaagagtgtttgggttcaccgtgtgtccaatgattctgtgcctcttgagctccaggaaagggctccctggtgatgcatgagatcttttcttggagtctctctgcagagttcactgggtttcctaaaggcaattcactatttcaaaagatggtgtgaggagcatgtggtgtccctaaaggagaattctgagccagggcacaaccactttatactgagctggatacactggtaggaatatactctgtcagctcagatagaaacctccctgcatggttggggctgggctgcagggggcgctccggatacacccagcacaggctcccgccccagagcaggtgcacaggaggctggggagaggttcctcccagggcctgggacttcctttaaaaatatctaaaataagtatttcacaaagactgctgaagtttgtataaatatctattcaattgtgagcatttatcaaactggatgttgtaatgagaaccacttttacaatggggatttcaaactctgctggaggtcaggaagagatcctttcttataaataaatgcaatttttggataaacacagtcattccctaaataacgcattcacatattatggtctagaaatgatgcaagttgaccctgagacagtcaaatgtggtttcaaagtgaggtgctgtccttgaggagcttgttctccagtgggggaagctctgtcaacacagagttcagggatgtgtaggggacacatggcctctaacaggattacggcttgaaccctcagcttctacagttgtgtcacccatgtgtctgtttctcatactgggtcaggaattgggctattaaatagcatccttcatgaatatgcaattaactgaggtgactatagtatctccgttccctgagagcctcacccaacaaccacacccctcctctggagaagcccctagatcacagctcctcaccatggactggacctgaaggatcctcttcttgatggcagcagcaacaggtaaggggctccccagtctcagggctgaggaagaaaccaggccagtcatgtgagacttcacccactcttgtgtccactccacaggtgcccactccctgcagctggtgcagtctgggcctgaggtgaagaagcctggggcctcagtgaaggtctcctataagtcttctggttacaccttcaccatctatggtatgaattgggtatgatagacccctggacagggctttgagtggatgtgatggatcatcacctacactgggaacccaacgtatacccacggcttcacaggatggtttgtcttctccatggacacgtctgtcagcacggcgtgtcttcagatcagcagcctaaaggctgaggacacggccgagtattactgtgcgaagtacacagtgtggaaacccacatcccgagagtttcagaaagcctgaggaaggaggcagctgtgctgagctgaggcagtggtacagcagttttctgaacttccatagtatctcattttgcattgagttccgctttaatattagccaagaatatgggatagacgggtgctcctaagagatccttaacttgcccattttgatgggttttcccaaagacgtgagaagccacttttttcgcaaagcatcccaaagccatgccctgctccagaaacacgtgtatccatttcctggtctttgattaactgacaaactctcatcagcgcacctgggctaatttcacatcaggtagaaatatgtgctttaaagcaaggctaacgttgtaatagcaattcctgcttaataaccttcagcattgttgttgtgtgctccatcaactaattacgttagttcaaggttctcaatgggagtttctaataaatataagggatgtatagaagttcccctaattaaaacaattgtgaagacaacctcagtgttcaaccatatttcaacccttcaccacaaaggaactttcatctctcctggaagttgggttcattttcaaattagtttttttattttaatatctcaagattattgtatgtgactattttagcagaaagtgaattatgggaacttgaactaaccaactgaaaatacattcagaactaattaaacaagatgccagaatgtgattggctccaggcattttaaattcaacaggttatgtaaccaggctttaaatttgcacatcttcgtgttaccttcatgacacagtcaactcccattatgtaagaaatggtgagtgcattcccaagggtcttgcacagttataaaaatagacttgatgaggtgaggagttgtttaaattcccctctgaagaagcagcatcaacccaacaaaccactctcttccctctgtgactagagctctgtcacaggccacatggacctaaatccttgatggagattacaggactacgtaaattggactgatcgtttttatgctgttaaattaataggtgagtctgcactccagcctgggcaacagaataatcttgtctgtaaaatacaaaagaaagataaattaatagatactgactttgacatttcggataataatattttcataaaccgaatttaattatacccacattgttacctacaccttcactgaaaagttcctagttatgttgagttccatcaacactccacatgttcaaatctggacatccaagagagtctagagaataaaacgcaatgagggcagtgaaacttgcgtatattcagcacctcttaactcaggaggactcaatacaccctggaacactctgcttttctgaatggctcacaatgactccagctcactctccaacctcctcaaacatctggcctctgtttgccctaagttcacgctctgctcttagtctatgttctgaagtctttgtagaggtgaaaatgagctgtcagatggatcttccttctcactgcaacatggaatttgctatttcacttaatgaccactctttccacaatggttgatttcttttggcctgttcattactggtgattttcaagggaatctcagttgaatctttactgttttgcattttgtctccatgacaatgttgggaagtttttcttctagcagcataacatgatctagtgacctgacacatttgcagcaaacaatacctacaaattcagaagctctttggttttctttccacgaaatataattcttgctcttctgtgtatgagcacatcctagcatccctgtacacacccaggtagatgtaaacacgccgatgaaatattccctgtaaataaaaaaagtatctcagtttctctcaatgttcataattctcctgagggtgaggaaggtacttctgggtctgctcaaacaaatggcccagagaccacctggtaggtaggtaaggagctcacctcgctctggatattgagtctgtctctttccctctgtcgtctcatagaaggccagcccacttgttcagctcctaagaagagagcccaggtttatccagattatacaacacaaccagcttctgatgactctcctgttacaacatccatggagatattttgtgtattatataattcaccaaactaatgtgaaatgcccaagttgcaatactgcacaccctagggtatgttcttgcaattcagcggaggagaaattctttcagagacagatggatctgaattggtaaatatgtgggtacgaattctgggtttgagtgtcattgtccagccatgtttcacaggtgtgacctgtcagggaagaaccagagttccttgttctctcagagggtagagctcacagaggtcctctctggttcccaggaaaggtaatttcactaatcttggtgatgagactatcctccagtgctgatgtactatagagttttcatctgaagctgtcactgctatccccaatgtacatcttttcacacagaaatgtttagaggtcaggccatattctcagggttacacattgagaaggatggagatatattctactaccttctcctgagatctcacacacaatctcaaatttcaaaaggtctcagaagggcagctctcaggtactatttaaaaataacccacttcctgggacaggtagcatccttctaaccatgatggatgttctgaactacagtacacattgcatggatccaggtttgtctcaattcactgtgattattacactcagcagctgtttcaatatgtctgaaggggtaaatgacaatttaggtgacctgggtgtatggttggtgttatatgaatctttaaatgtagaacagtattaactgtattccaaaatctgtctttgatccatgatcacacttgtctcccagaccagctccttcagcacatttcctacctggaagaagaggactctgggtttggtgaggggaggccacaggaagagaactgagttctcagagggcacagccagcatacacctcccagggtgagcccaaaagactggggcctccctcatccctttttacctatccatacaaaggcaccacccacatgcaaatcctcacttaggcacccacaggaaatgactacacatttccttaaattcagggtccagctcacatgggaagtgctttctgagagtcatggacctcctgcacaagaacggtaccgggctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccac

SEQ ID NO: 122 - DNA, 2570 bp, Artificial Sequence
  Region of SEQ ID NO121 that is integrated
ggtaccgggctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtgggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagcc

SEQ ID NO: 123 - DNA, 1131 bp, Artificial Sequence
  Parental 3kb15a-7-4Tdelta Mlu I-Nae I reference sequence
gcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtgggtgtcacgctcgtcgtttggtatggcttcattcagctccggttcccaacgatcaaggcgagttacatgaa

SEQ ID NO: 124 - DNA, 29 bp, Artificial Sequence
  Oligonucleotide primer
gagggttaattgcgcgcttggcgtaatca

SEQ ID NO: 125 - DNA, 31 bp, Artificial Sequence
  Oligonucleotide primer
agatgctgaagatcagttgggtgcacgagtg

SEQ ID NO: 126 - DNA, 34 bp, Artificial Sequence
  Oligonucleotide primer
gttatccgctcacaattccacacaacatacgagc

SEQ ID NO: 127 - DNA, 33 bp, Artificial Sequence
  Oligonucleotide primer
agttctgctatgtggcgcggtattatcccgtat

SEQ ID NO: 128 - DNA, 16 bp, Artificial Sequence
  Oligonucleotide primer
ggccgcgttgctggcg

SEQ ID NO: 129 - DNA, 39 bp, Artificial Sequence; identical to SEQ ID NO: 73
  Oligonucleotide primer

SEQ ID NO: 130 - DNA, 3786 bp, Artificial Sequence
  Plasmid pME18sEGPFCD26
aagcttggctgtggaatgtgtgtcagttagggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctcctcgatcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctctgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattcatgaagacaccgtggaaggttctcctgggactgctgggtgctgctgcgcttgtcaccatcatcaccgtgcccgtggttctgctgaacaaaaccggtatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagaggatccgggcccagggcgccgattataaagatgatgatgataaagattataaagatgatgatgataaataataaactagtctagagaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatccccgggtaccgagctcgaattaattcctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgc

(19) United States
(12) Patent Application Publication
(10) Pub. No.: US 2006/0099611 A1
(43) Pub. Date: May 11, 2006

(54) In vivo affinity maturation scheme

(75) Inventors: Robert Irving, Pakenham (AU); Peter J. Hudson, Blackburn (AU); Huseyin Mustafa, Glenroy (AU); Kim Wark, St. Kilda (AU); Matias E. Abregu, Sunshine (AU)

Correspondence Address: MARSHALL, GERSTEIN & BORUN LLP, 233 S. WACKER DRIVE, SUITE 6300, SEARS TOWER, CHICAGO, IL, 60606, US

(73) Assignee: DIATECH PTY LTD, Brisbane (AU)

(21) Appl. No.: 11/156,014

(22) Filed: Jun. 17, 2005

Related U.S. Application Data
(63) Continuation-in-part of application No. PCT/AU03/01697, filed on Dec. 18, 2003.

(30) Foreign Application Priority Data
Dec. 18, 2002 (AU) 2002953381

(51) Int. Cl. C12Q 1/68; C12P 21/06; C12N 1/21; C12N 1/18; C12N 5/06

(52) U.S. Cl. 435/6; 435/69.1; 435/455; 435/252.3; 435/254.2; 435/348; 435/325

(57) ABSTRACT

The present invention relates to the field of evolution of nucleic acids in vivo and provides methods and compositions for introducing diversity into gene products. The present invention allows generation of new sequences that have desirable properties by virtue of high frequency mutation events within a cell. The high frequency mutation of a polynucleotide sequence results in, the production of a large population of new sequence variants. Appropriate selection and/or screening permits identification and isolation of mutant forms of the polynucleotide sequence as well as products resulting from expression of the mutant sequences.

### FIELD OF THE INVENTION

The present invention relates to the field of evolution of nucleic acids in vivo and provides methods and compositions for introducing diversity into gene products. The present invention allows generation of new sequences that have desirable properties by virtue of high frequency mutation events within a cell. The high frequency mutation of a polynucleotide sequence results in the production of a large population of new sequence variants. Appropriate selection and/or screening permits identification and isolation of mutant forms of the polynucleotide sequence as well as products resulting from expression of the mutant sequences.

### BACKGROUND OF THE INVENTION

In vitro evolution of proteins involves generating diversity by introducing mutations into known gene sequences to produce a library of mutant sequences, translating the sequences to produce a very large number of mutant gene products, which are then selected for the desired properties. Such schemes can be divided into two distinct groups; i) partial in vitro methods in which the mutated target protein is displayed on the surface of the either phage, bacteria or yeast but the mutation step is performed outside the cell and ii) entirely in vitro methods in which the target is displayed as part of a ribosome quaternary complex or polysome and all other steps including mutation are performed in a cell free environment. These processes have the potential for generating proteins with improved diagnostic and therapeutic utilities. Unfortunately, however, the potential of such processes has been limited by deficiencies in methods currently available for mutation, library generation and display of correctly folded proteins.

For example, in the case of partial in vitro methods, the DNA must be synthesised in vitro or extracted from the cells for mutagenesis. Although various mutagenesis approaches (including error prone PCR, DNA shuffling, chain shuffling and site directed mutagenesis) have been successfully used to generate mutant libraries, some of this diversity is lost due to limitations in subsequent transformation efficiency. Consequently, the generation of large libraries (e.g. beyond a library size of 10¹⁰) of unique individual genes and their encoded proteins has proven difficult particularly with phage display systems. A further disadvantage is that methods which utilise phage display systems require several sequential steps of mutation, amplification, selection and further mutation. Given that extraction and reintroduction of DNA into the cell is required for these systems, their potential to generate large diversity in the target gene library is further restricted.

To circumvent this problem, entirely in vitro methods such as continuous in vitro evolution (CIVE) have been developed and are described, for example, in WO 99/58661. In theory any diversity created through mutagenesis in these systems is not lost. In the case of CIVE, a mutating enzyme is used to introduce nucleic acid base changes into the target sequence. The only factor limiting diversity here is the mutation rate of this enzyme. Reports of mutation rates using other in vitro mutation methods such as error prone PCR, DNA shuffling (sexual PCR), chain shuffling and site directed mutagenesis over selected CDRs which can be used in this scheme vary significantly. Despite using different mechanisms all these approaches operate in an artificial environment in which only defined components required for these processes are present. It is possible there may be additional unknown factors involved, which are not supplied. Furthermore, this cell-free environment lacks the secretory and post-translational machinery required to produce a correctly folded and processed protein. As a result, this restricts the type of targets which can be “evolved” in these systems and allows incorrectly folded, unmodified mutant proteins which have no functional relevance in a clinical setting to be selected. Bacterial and phage display also have the same associated problems.

In vivo evolution of proteins involves the same steps and principles as previously described for in vitro evolution (i.e. mutation, display, selection and amplification). However, in vivo systems overcome many of the problems associated with the in vitro approaches. One in vivo cyclical procedure that has been reported involves Escherichia coli mutator cells that were used as a vehicle for mutation of recombinant antibody genes. The E. coli mutator cells, MUTD5-HIT which carried a mutated DNAQ gene were used as the source of the S-30 extracts and therefore allowed mutations to be introduced into DNA during replication as a result of proofreading errors. However, mutation rates were low compared to the required rate. For example, to mutate 20 residues with the complete permutation of 20 amino acid requires a library size of 1×10²⁶, an extremely difficult task with currently available phage library display methodology. Obviously, the disadvantages with using bacteria and phage in terms of transformation efficiencies, and protein folding etc. make this a less desirable scheme.

In view of the above it is clear that the current affinity maturation schemes are somewhat limited in their ability to generate and select functionally superior binders.

### SUMMARY OF THE INVENTION

The present inventors have now developed a novel process for the in vivo evolution of gene products. This process generates mutants of target nucleic acid sequences by somatic hypermutation, yielding mutant products capable of undergoing any post-translational modifications that may be required for biological activity. A selection system particular to the properties of the target product is then utilized to identify desirable mutants.

Accordingly, in a first aspect, the present invention provides a method for producing and selecting a gene product with desired characteristics, the method comprising
(i) introducing into a hypermutating cell a target nucleic acid molecule encoding a gene product such that the target nucleic acid molecule is integrated into an immunoglobulin locus of the genome of the hypermutating cell;
(ii) culturing the hypermutating cell such that the target nucleic acid molecule undergoes hypermutation during DNA and/or RNA synthesis, giving rise to a population of cells expressing mutant gene products; and
(iii) selecting a mutant gene product with desired characteristics.

The term “immunoglobulin locus” refers to a variable region of an antibody molecule or all or a portion of a regulatory nucleotide sequence that controls expression of an antibody molecule. Immunoglobulin loci for heavy chains may include but are not limited to all or a portion of the V, D, J, and switch regions (including intervening sequences called introns) and flanking sequences associated with or adjacent to the particular heavy chain constant region gene expressed by the antibody-producing cell to be transfected and may include regions located within or downstream of the constant region (including introns). Immunoglobulin loci for light chains may include but are not limited to the V and J regions, their upstream flanking sequences, and intervening sequences (introns), associated with or adjacent to the light chain constant region gene expressed by the antibody-producing cell to be transfected and may include regions located within or downstream of the constant region (including introns). Immunoglobulin loci for heavy chain variable regions may include but are not limited to all or a portion of the V, D, and J regions (including introns) and flanking sequences associated with or adjacent to the particular variable region gene expressed by the antibody-producing cell to be transfected. Immunoglobulin loci for light chain variable regions may include but are not limited to the V and J region (including introns) and flanking sequences associated with or adjacent to the light chain variable region gene expressed by the antibody-producing cell to be transfected.

In the human, the immunoglobulin heavy chain (IgH) locus is located on chromosome 14. In the 5′-3′ direction of transcription, the locus comprises a large cluster of variable region genes (V_{H}), the diversity (D) region genes, followed by the joining (J_{H}) region genes and the constant (C_{H}) gene cluster. The size of the locus is estimated to be about from 1,500 to about 2,500 kilobases (kb). During B-cell development, discontinuous gene segments from the germ line IgH locus are juxtaposed by means of a physical rearrangement of the DNA. In order for a functional heavy chain Ig polypeptide to be produced, three discontinuous DNA segments, from the V_{H}, D, and J_{H} regions must be joined in a specific sequential fashion; first D to J_{H} then V_{H} to DJ_{H}, generating the functional unit V_{H}DJ_{H}. Once a V_{H}DJ_{H} has been formed, specific heavy chains are produced following transcription of the Ig locus, utilizing as a template the specific V_{H}DJ_{H}C_{H} unit comprising exons and introns.

There are two loci for immunoglobulin light chains (IgL), the kappa locus on human chromosome 2 and the lambda locus on human chromosome 22. The organization of the IgL loci is similar to that of the IgH locus, except that the D region is not present. Following IgH rearrangement, rearrangement of a light chain locus is similarly accomplished by V_{L} to J_{L} joining of the kappa or lambda chain. The sizes of the lambda and kappa loci are each approximately 1000 kb to 2000 kb. Expression of rearranged IgH and an Ig kappa or Ig lambda light chain in a particular B-cell allows for the generation of antibody molecules.

In a further preferred embodiment of the invention the immunoglobulin locus is a rearranged V_{H}4 gene. In a further preferred embodiment, the immunoglobulin locus is a rearranged VH₄₋₃₄ allele.

By “hypermutation” we mean a mechanism by which mutagenesis occurs at a rate approaching that naturally occurring in the immunoglobulin variable region, which is preferably in the range of 10⁻⁴ to 10⁻³/base pair/generation/cell but more preferably in the range of 5×10⁻⁵ to 5×10⁻⁴/base pair/generation/cell.

A “hypermutating cell” is a cell or cell line containing hypermutation elements.

By “hypermutation elements” we mean an intronic enhancer (Ei), matrix attachment regions (MAR), and a 3′ enhancer. The intronic enhancer may be, for example, E mu or E kappa. The 3′ enhancer may be,, for example, a 3′ kappa enhancer or a 3′H enhancer.

A “matrix attachment region” (MAR) is defined by its ability to bind to the nuclear matrix. Matrix attachment region sequences flank the IgH intronic enhancer.

In one embodiment the hypermutating cell is an immunoglobulin-expressing cell which is capable of expressing at least one immunoglobulin V gene. A V gene may be a variable light chain (V_{L}) or a variable heavy chain (V_{H}) gene, and may be produced as part of an entire immunoglobulin molecule. Preferred hypermutating cells for use in the present invention are derived from B-cell lines. Lymphoma cells may be used for the isolation of constitutively hypermutating cell lines for use in the present invention.

In a preferred embodiment of the present invention, following integration into the immunoglobulin locus of the hypermutating cell, the target nucleic acid molecule is located in proximity to one or more endogenous hypermutation elements. Preferably, the immunoglobulin locus is a V_{H} gene and the target nucleic acid molecule is located in proximity to an endogenous intronic enhancer, and endogenous matrix attachment regions.

The phrase “located in proximity to” means that hypermutation elements are located close enough to the target nucleic acid molecule to effect hypermutation of the target nucleic acid molecule.

In an alternative embodiment of the invention, following integration into the immunoglobulin locus of the hypermutating cell, the target nucleic acid molecule is located in proximity to at least one exogenous hypermutation element. For example, the target nucleic acid molecule may be located in proximity to an exogenous intronic enhancer, an exogenous matrix attachment region and/or an exogenous 3′ kappa enhancer. Any one or more of these exogenous elements may be integrated into the immunoglobulin locus simultaneously with the target nucleic acid molecule.

A suitable exogenous “intronic enhancer” may be, for example, the Xbal-EcoRI fragment described in Grosschedl et al (1985) Cell Vol 41:885-897, the intronic enhancer described in Rabbitts et al (1983) Nature 306 (5945):806-809, or the intronic enhancer described in Ravetch et al (1981) Cell 27 (3 Pt 2): 583-591; or can be one or more sub-fragments thereof determined to have hypermutation activity.

A suitable exogenous “3′ kappa enhancer” is the ScaI-XbaI fragment described in Meyer et al (1989) EMBO Journal Vol. 8, no. 7 p. 1959-1964 and can be one or more sub-fragments determined to have hypermutation activity.

Hypermutation-competent fragments of exogenous intronic enhancers or the 3′ kappa enhancer can be identified in a number of ways. One way is to perform deletional analysis by constructing hypermutation cassettes containing various enhancer deletion mutants and a reporter gene. The hypermutation efficency of the enhancer deletion mutant can be assessed by determining the rate of mutation of the reporter gene. Deletion mutants can be prepared in a variety of ways. Oligonucleotides can be designed containing fragment sequences to be tested. Alternatively, a more random approach is to linearize the expression vector by restriction digest within an enhancer, followed by subsequent exonuclease treatment and religation. Yet another method is to simply use restriction digests to remove sections of DNA.

It is preferred that following integration, a 3′ enhancer and/or an intronic enhancer are positioned at a location 3′ of the target nucleic acid sequence. It is further preferred that the intronic enhancer be located in greater proximity to the target gene than the 3′ enhancer. The 5′ end of the intronic enhancer is preferably positioned up to 3 kb 3′ of the 3′ end of the target gene, preferably less than 2 kb, more preferably less than 1 kb, and most preferably immediately adjacent to the target nucleic acid sequence. The intronic enhancer can be positioned greater than 3 kb 3′ of the target gene, but this is less preferred. The 3′ enhancer is preferably located up to 20 kb and preferably 5-15 kb 3′ of the intronic enhancer. The 3′ enhancer can be located as close as 1 kb 3′ of the intronic enhancer, but this is less preferred. In another embodiment, the 3′ enhancer fragment is located 5′ relative to the target gene. The intronic enhancer can also be positioned 5′ relative to the target gene, although this embodiment is less preferred.

In a further preferred embodiment, the enhancers are present in a genomic orientation. The enhancer sequence present in the genomic immunoglobulin gene is present in a “genomic orientation”. If it is flipped in the construct so that it now appears in a 3′ to 5′ orientation (as opposed to the 5′ to 3′ orientation in the native genomic configuration), it is present in the “reverse orientation”. However, the 3′ enhancer can be present in reverse orientation. The enhancer can also be present in reverse orientation, but this is less preferred.

In a further preferred embodiment of the first aspect, following integration of the target nucleic acid molecule into the immunoglobulin locus, the target nucleic acid molecule is operatively linked to a promoter. Preferably, the target nucleic acid molecule is located downstream of the promoter and upstream of an intronic enhancer.

The term “promoter” is well-known in the art and encompasses nucleic acid regions ranging in size and complexity from minimal promoters to promoters including upstream elements and enhancers.

In one preferred embodiment of the invention, the promoter is a naturally occurring promoter that exists within the immunoglobulin locus. In one embodiment, the promoter is an immunoglobulin heavy or light chain promoter. Preferably, the promoter is an immunoglobulin heavy chain promoter of a V_{H}4 allele. There is strong conservation between the promoter sequences of the V_{H}4 alleles (FIG. 1). A wide range of hypermutating cell lines (including RAMOS and BL2 cell lines) carry rearranged V_{H}4 alleles.

Alternatively, the promoter may be a heterologous or exogenous promoter selected from those which are functional in mammalian cells, although prokaryotic promoters and promoters functional in other eukaryotic cells may be used. The promoter may be derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of α-actin, β-actin, tubulin) or, alternatively, a tissue-specific manner (such as promoters of the genes for pyruvate kinase). They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter. In a preferred embodiment, the promoter is an immunoglobulin promoter sequence such as a murine immunoglobulin promoter sequence or a human immunoglobulin heavy chain promoter.

The promoter region of mammalian/human genes can contain several regulatory elements in the DNA sequences, and span several hundred bases or more, it is generally observed that one of these elements, designated “TATA box” sequence, in eukaryotic promoter regions is usually found approximately 300 bases or more upstream of the translation initiation site (start) sequence, ATG. Rearrangements which bring the V gene promoter into closer proximity to the ATG translation start signal also brings the promoter closer to the enhancer which is 3′ and located in the C region. This activates the promoter which indicates that the close proximity to the enhancer may affect the rate at which the DNA in the VH locus is mutated.

The present inventors have found that in a RAMOS RA-1 cell line in which the rearrangement generates a functional VH4-34 allele, the promoter is significantly closer to the translation initiation start site than in other mammalian/human genes (based upon the number of nucleotides between the 3′ end of the “TATA box” and the initiation codon of the immunoglobulin leader sequence). This proximity of the promoter to the start codon and the three dimensional structure of the promoter caused significant difficulties in cloning this region from RAMOS RA-1.

Accordingly, in a further preferred embodiment of the invention, following integration of the target nucleic acid molecule into the immunoglobulin locus, the target nucleic acid molecule is located within close proximity to the promoter. Preferably, the initiation codon of the target nucleic acid molecule is located within 500 bp of the 3′ end of the promoter, more preferably within 200 bp of the 3′ end of the promoter, more preferably within 100 bp of the 3′ end of the promoter and more preferably within 20 bp of the 3′ end of the promoter.

It may also be advantageous for the promoters to be inducible so that the levels of expression of the heterologous gene can be regulated during the life-time of the cell. Inducible means that the levels of expression obtained using the promoter can be regulated.

In addition, any of these promoters may be modified by the addition of further regulatory sequences, for example enhancer sequences. Chimeric promoters may also be used comprising sequence elements from two or more different promoters described above.

In a further preferred embodiment of the present invention, the target nucleic acid molecule is introduced into the cell by way of an integration vector comprising a sequence homologous to a region of at least 500 bp, more preferably at least 2 kb, more preferably approximately 5 kb upstream of a rearranged V gene and a sequence homologous to a region of at least 500 bp, more preferably at least 2 kb, more preferably approximately 5 kb downstream of the same rearranged V gene. Preferably, the sequence homologous to a region downstream of the rearranged V gene comprises an intronic enhancer and matrix attachment regions or portions thereof. It is preferred that the upstream and downstream homologous sequences are at least 500 bp in length, more preferably about 5-kb in length.

A “target nucleic acid molecule” can be any nucleic acid molecule of interest (including DNA and RNA molecules) encoding a gene product where diversification of the gene product is desired.

The “gene product” may be any biologically active molecule of interest. For example, the gene product may be a catalytic molecule such as a ribozyme, a DNAzyme, an LNAzyme or an RNAi/siRNA molecule. Alternatively, the gene product may be an antibody or fragment thereof, an enzyme, a hormone, a receptor, a cell surface molecule, a viral protein, transcription factor or any other biologically active polypeptide.

The selected mutant target sequence may be recycled through the methods of the present invention in order to introduce further diversification. Accordingly, in a further preferred embodiment of the first and second aspects, at least steps (ii) and (iii) of the method are repeated.

The hypermutating cell may be a mammalian, avian, yeast, fungi, insect or bacterial cell. In a preferred embodiment, the hypermutating cell is a mammalian cell. The mammalian hypermutating cell may be selected from the group consisting of RAMOS, BL2, BL41, BL70 and Nalm.

The method of the present invention may include further steps to increase the rate of mutation of the target nucleic acid sequence. For example, the hypermutating cell may be cultured in the presence of chemical mutagens. Suitable chemical mutagens include, for example, sodium bisulfite, nitrous acid, hydroxylamine, hydrazine or formic acid. Other agents which are analogues of nucleotide or nucleoside precursors include nitrosoguanidine, ribavirin, 5-bromouracil, 2-aminopurine, 5-formyl uridine, isoguanosine, acridine and of N⁴-aminocytidine, N¹-methyl-N⁴-aminocytidine, 3,N⁴-ethenocytidine, 3-methylcytidine, 5-hydroxycytidine, N⁴-dimethylcytidine, 5-(2-hydroxyethyl)cytidine, 5-chlorocytidine, 5-bromocytidine, N⁴-methyl-N.sup.4-aminocytidine, 5-aminocytidine, 5-nitrosocytidine, 5-(hydroxyalkyl)-cytidine, 5-(thioalkyl)-cytidine and cytidine glycol, 5-hydroxyuridine, 3-hydroxyethyluridine, 3-methyluridine, O²-methyluridine, O²-ethyluridine, 5-aminouridine, O⁴-methyluridine, O⁴-ethyluridine, O⁴-isobutyluridine, O⁴-alkyluridine, 5-nitrosouridine, 5-(hydroxyalkyl)-uridine, and 5-(thioalkyl)-uridine, 1,N⁶-ethenoadenosine, 3-methyladenosine, and N⁶-methyladenosine, 8-hydroxyguanosine, O⁶-methylguanosine, O⁶-ethylguanosine, O⁶-isopropylguanosine, 3,N²-ethenoguanosine, O⁶-alkylguanosine, 8-oxo-guanosine, 2,N³-ethenoguanosine, and 8-aminoguanosineas well as derivatives/analogues thereof. Examples of suitable nucleoside precursors, and synthesis thereof, are described in further detail in USSN 20030119764. Generally, these agents are added to the replication or transcription reaction thereby mutating the sequence. Intercalating agents such as proflavine, acriflavine, quinacrine and the like can also be used.

Random mutagenesis of the target nucleic acid molecule can also be achieved by irradiation with X-rays or ultraviolet light.

Antigen stimulation of the hypermutating cells, or exposure of the cells to interleukins (such as IL-2, IL4 or IL-10) or CD40 ligand or B cell activating factor (BAAFF) may also be used to increase the mutation frequency.

In one preferred embodiment, the level or activity of activation-induced cytidine deaminase (AID) within the hypermutating cell is increased. This may be achieved, for example, by increasing expression levels of AID within the hypermutating cells. For example, the hypermutating cells may be transfected with plasmid vectors which encode and express AID.

It will be appreciated by those skilled in the art that any process of selecting a mutant product can be used in the methods of the present invention.

In one embodiment, selection can be achieved by binding to a target molecule or by measurement of a biological response affected by the mutant product.

In another example, if the product of interest is an agent that promotes or reduces cell growth or division, the selection process can involve exposing mutant products to a population of cells and monitoring the biological responses of those cells.

In another example, if the mutant product is a receptor ligand, the process can involve exposing mutant proteins to cells expressing the receptor and monitoring a biological response effected by signalling of the receptor.

In one embodiment, the mutant product is selected by way of an assay performed within the hypermutating cell. For example, if the target nucleotide sequence encodes an enzyme, the assay may simply measure enzymatic activity.

Alternatively , the assay performed within the hypermutating cell may be a protein-fragment complementation assay (PCA). PCAs rely on the complementation of enzyme fragments fused to interacting proteins that reconstitute enzymatic activity once dimerised. Examples of PCA protocols are described in Michnick (2001) Current opinion in structural biology 11:472-477; Wehrman et al. (2002). PNAS 99(6):3469-3474; and Galarneau et al. (2002). Nature 20:616-622.

Suitable enzyme fragments may be derived, for example, from β-lactamase. Studies using the β-lactamase system in both bacteria and mammalian cells have successfully validated it as a suitable reporter system to detect protein-protein interactions inside the cell.

The target nucleic acid molecule may therefore encode a fusion protein comprising a binding partner and a β-lactamase fragment. Its binding partner may be introduced into the cell as a fusion protein comprising a complementary β-lactamase fragment. Selection occurs inside the cell with the binding of the target protein to its cognate partner which is detected by β-lactamase activity on a substrate supplied.

If selection assays such as PCAs are used, it is not necessary to display the target polypeptide on the surface of the hypermutating cell. These selection assays are particularly advantageous for targets which are naturally found intracellularly.

In an alternative embodiment of the present invention, the target nucleic acid molecule is linked to a sequence encoding an anchor domain such that following expression, the mutant gene product is displayed on the surface of the hypermutating cell. Examples of suitable anchor domains include attachment signals from glycosylphosphatidylinositol (GPI) anchored membrane proteins or transmembrane domains of other cell surface proteins.

If the gene product to be displayed is not normally found on the cell surface, it is preferred that the target nucleic acid molecule is also linked to a sequence encoding an N-terminal signal peptide (or a leader peptide). This signal peptide facilitates targeting of the gene product to the plasma membrane.

Following display on the surface of the hypermutating cell, the mutant gene product may be selected by detecting binding of a binding partner to the mutant gene product. This may involve, for example, labelling cells with a detectable marker such as a fluorescent dye and allowing binding to occur between the mutant protein on the cell surface and its binding partner. If the binding partner has been immobilized on a plate, a suitable detection system, such as a fluorimeter, can be used to identify wells containing a mutant of interest.

Alternatively, the binding partner may be labelled with a fluorescent tag, and cells expressing a mutant gene product of interest may be sorted using flow cytometric techniques. The binding partner may be selected from the group consisting of an antibody, receptor, hormone, enzyme, cell surface molecule, transcription factor, DNA or RNA molecule.

In another embodiment of the invention, the target gene product is expressed in soluble form.

To prevent repeated mutation after selection, hypermutation may be arrested prior to culturing the selected cells. This can be accomplished in a number of ways, including fusion to a myeloma or repression of an inducible promoter.

In a further preferred embodiment of the invention, the mutant nucleic acid sequence encoding the selected gene product is recovered from the hypermutating cell. This recovery can be achieved by amplification of the mutant nucleic acid sequence in whole or in part by polymerase chain reaction, using oligonucleotides that will anneal to locations outside the region of hypermutation or within the target sequence itself. Alternatively, the mutant nucleic acid sequence may be amplified using RT-PCR. The mutant nucleic acid sequence can then be subcloned for other purposes, such as expression, purification, or characterization.

The conditioned media from the transfected cells can be concentrated if desired and applied to the selection system. Specific binders can be identified directly or indirectly, for example by antibody recognition of either the target gene product itself or an attached tag sequence. The mutant gene products of interest can then be further characterized by a number of protein chemistry techniques such as micro-sequencing.

In a second aspect the present invention provides a gene product produced by a method of the first aspect.

In a third aspect the present invention provides a vector for targeted integration into an immunoglobulin locus of a hypermutating cell, the vector comprising a sequence homologous to a region upstream of a rearranged V gene of the hypermutating cell, a sequence homologous to a region downstream of a rearranged V gene of the hypermutating cell and a site for insertion of a target nucleic acid molecule.

In a preferred embodiment of the third aspect, the region upstream of the rearranged VH gene of the-hypermutating cell is a region within nucleotides 1 to 5190 of SEQ ID NO:1. Preferably, the region is at least 500 bp, more preferably at least 2 kb, more preferably about 5 kb within nucleotides 1 to 5190 of SEQ ID NO:1. In a further preferred embodiment the region upstream of the rearranged V gene of the hypermutating cell comprises nucleotides 191 to 5190 of SEQ ID NO:1.

In a further preferred embodiment of the third aspect, the region downstream of the rearranged VH gene of the hypermutating cell is a region within nucleotides 5709 to 8699 of SEQ ID NO:1. Preferably, the region is at least 500 bp, more preferably at least 3 kb, 5709 to 8699 of SEQ ID NO:1. In a further preferred embodiment the region downstream of the rearranged VH gene of the hypermutating cell comprises nucleotides 5709 to 8634 of SEQ ID NO:1.

In a further preferred embodiment of the third aspect, the vector further comprises a selectable marker. The term “selection marker” or “selectable marker” includes both positive and negative selection markers. A “positive selection marker” is a nucleic acid sequence that allows the survival of cells containing the positive selection marker under growth conditions that kill or prevent growth of cells lacking the marker. An example of a positive selection marker is a nucleic acid sequence which promotes expression of the neomycin resistance gene, or the kanamycin resistance gene. Cells not containing the neomycin resistance gene are selected against by application of G418, whereas cells expressing the neomycin resistance gene are not harmed by G418 (positive selection). A “negative selection marker” is a nucleic acid sequence that kills, prevents growth of or otherwise selects against cells containing the negative selection marker, usually upon application of an appropriate exogenous agent. An example of a negative selection marker is a nucleic acid sequence which promotes expression of the thymidine kinase gene of herpes simplex virus (HSV-TK). Cells expressing HSV-TK are selected against by application of ganciclovir or 1-2′-deoxy-2′fluoro-b-D-arabinofuranosyl-5-iodouracil (FIAU); negative selection), whereas cells not expressing the gene are relatively unharmed by ganciclovir or FIAU.

In a further preferred embodiment the vector encodes an anchor molecule suitable for display of the protein encoded by the target nucleic acid molecule.

In a further preferred embodiment the vector comprises a target nucleic acid molecule with an epitope tag(s) (for example, two flag tags).

In a further preferred embodiment, the vector for targeted integration comprises a sequence as set out in SEQ ID NO:110.

The present invention provides a novel approach for generating diversity in a gene product (see FIG. 2). An important feature of the present invention is that the target nucleic acid molecule is integrated into the immunoglobulin locus of a host cell genome. This integration is achieved by including sequences homologous to regions upstream and downstream of the rearranged V gene in the integration vector. This directed integration ensures that the target nucleic acid molecule is positioned in proximity to elements that effect hypermutation (e.g. in proximity to elements such as an intronic enhancer, matrix attachment regions and/or a 3′ enhancer) following integration.

The integration process also ensures that only one copy of the target nucleic acid molecule is present in each cell. This facilitates the recovery of mutant target nucleic acid sequences of interest following the selection process by, for example, PCR or RT-PCR. In contrast, methods which involve the introduction of a target nucleic acid on a self replication vector or on a vector which integrates randomly into the host genome are likely to result in multiple mutated copies of target nucleic acid molecule in the host cell. This makes it difficult to determine which sequence encodes the mutant gene product which has been selected for its desired properties.

It will be appreciated that the methods of the present invention may be used for a variety of purposes. For example, the methods of the present invention can be used to effect affinity maturation of antibodies. In one aspect, the invention may be applied toward improving the affinity of antibodies from “naive,” i.e., non-immune, phage human antibody libraries. Such libraries already exist and yield antibodies to any antigen. However, since they are made from non-immunized individuals, their affinities are low. In another aspect of the invention, the affinity of antibodies that were generated by conventional hybridoma techniques can be improved by applying a high rate mutagenesis system of the invention to the isolated target nucleic acid encoding for the initial low-affinity antibody. These enhanced-affinity antibodies can be utilized as improvements over many antibody-based diagnostics and therapeutics currently available.

The methods of the present invention allow a very large library of peptides and single-chain antibodies to be screened and the polynucleotide sequence encoding the desired peptide(s) or single-chain antibodies to be selected. The pool of polynucleotides can then be isolated and shuffled to recombine combinatorially the amino acid sequence of the selected peptide(s) (or predetermined portions thereof) or single-chain antibodies (or just V_{H}, V_{L}, or CDR portions thereof). Using these methods, one can identify a peptide or single-chain antibody as having a desired binding affinity for a molecule and can exploit the process of the invention to converge rapidly to a desired high-affinity peptide or scFv. The peptide or antibody can then be synthesized in bulk by conventional means for any suitable use (e.g., as a therapeutic or diagnostic agent).

The mutagenesis system can also be used to effect receptor or ligand modification. In one aspect, the invention can generate a ligand or receptor with enhanced binding V characteristics for its corresponding receptor or ligand. In another aspect, the mutagenesis system can be used to generate an inhibitor of functional receptor-ligand interaction by creating a ligand or receptor that still binds, but does not elicit a functional response. In yet another aspect of the invention, multiple biologically active variants of a target protein can be identified and recovered, thereby, providing a means to study structure-function relationships of the protein. Additionally, species diversity can be investigated by comparing results obtained by selections utilizing receptors or other molecules from different species.

A receptor or ligand can be modified such that it can still bind, but does not signal any more. Alternatively, a better signalling ligand can be selected, which would provide a lower effective dosage of a pharmacologically active therapeutic.

The mutagenesis system of the present invention may also be used, for example, on a target such as caspase, an initiation factor target involved in a novel survival mechanism. This involves a cascade of essentially signalling reactions on the route to programmed cell death (apoptosis). Caspase-3 once activated binds to, and cleaves (activates) the ‘cell death’ proteins (including Id3). In vivo mutation and expression of mutated caspases, especially caspase-3, would have an effect on apoptosis. Therefore caspase 3 would be a preferred target molecule. This could be relevant to diagnostics in cell signal transduction for monitoring and detection of cancer, and with potential therapeutic outcomes.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### A BRIEF DESCRIPTION OF THE FIGURES

FIG. 1: Alignment of human immunoglobulin heavy chain promoters for VH4 alleles. Sequences provided as SEQ ID NO's 2 to 7.

FIG. 2: Schematic representation of a preferred method of the present invention.

FIG. 3: Schematic representation of the gene targeting region—the preferred site for direct insertion of the target nucleic acid into a rearranged V gene.

FIG. 4: Schematic representation of vector 3 kb15a-7-4T.

FIG. 5: Schematic representation of vector KW2.

FIG. 6: Schematic representation of vector for targeted integration, KW3.

FIG. 7: RAMOS cells stained with CFSE showing successive divisions on each day.

FIG. 8: Schematic representation of vector pME18SasFP499.

FIG. 9: The effect of DNA amount and electroporation parameters on transfection of RAMOS RA-1 cells with pME18SEGFP.

FIG. 10: Comparison of RAMOS RA-1 cells transfected with pME18sEGFP, pME18sasFP499 or mock transfected.

FIG. 11: (A): Quantum Simply Cellular Beads stained with mouse anti-human IgM-Alexa 488. (B) RAMOS cells stained with mouse anti-human IgM-Alexa 488. (C) Quantitation of IgM molecules on the surface of RAMOS RA-1 cells.

FIG. 12: Comparison of IgM expression on RAMOS RA-1 cells of different passage number. (A) RAMOS RA-1 passage 1 (B) RAMOS RA-1 passage 14.

FIG. 13: IgM expression on RAMOS RA-1 samples from different sources.

FIG. 14: Schematic representation of sequential overlap extension PCR.

FIG. 15: Schematic representation of mammalian expression vector pME18sCD26asfp499.

FIG. 16: Comparison of expression of asFP499 with and without the CD26 anchor in RAMOS RA-1.

FIG. 17: Comparison of expression of asFP499 with and without the CD26 anchor in HEK 293 T.

FIG. 18. Structure of plasmid 3 kb15a-7-4TΔMluI-NaeI.

FIG. 19. Enrichment and sorting protocol used in Example 13.

FIG. 20. Frequency of mutated sequences in three independent experiments.

FIG. 21. Frequency of transitions and transversions in three independent experiments.

FIG. 22. Spectra of nucleotide substitutions.

FIG. 23. Distribution of base changes in the amplified region of foreign integrated sequence.

FIG. 24. Mutation frequency in relation to hotspot motif frequency. Near hotspot defined as 5 or less bases upstream or downstream of the hotspot motif.

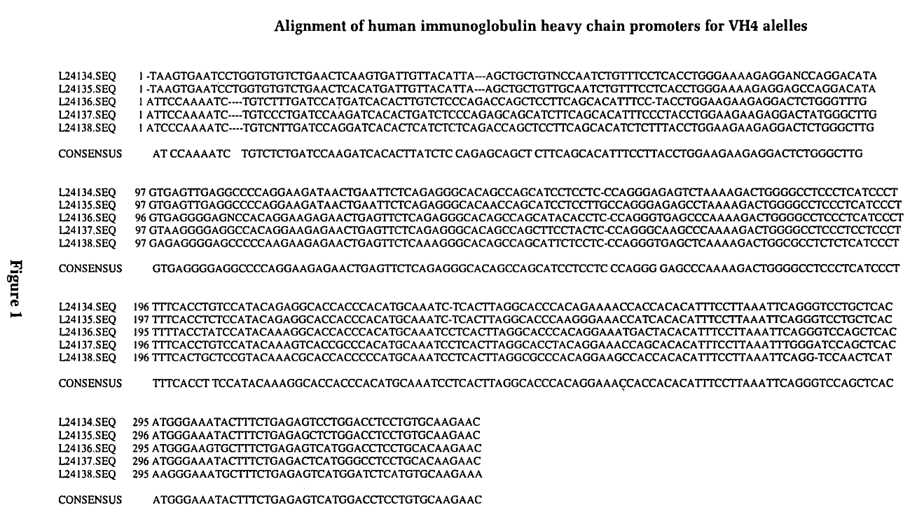

### KEY TO THE SEQUENCE LISTING

SEQ ID NO:1—Heavy chain locus of Ramos RA-1 cells.

SEQ ID NO:2—Sequence of promoter region of a VH4 allele.

SEQ ID NO:3—Sequence of promoter region of a VH4 allele.

SEQ ID NO:4—Sequence of promoter region of a VH4 allele.

SEQ ID NO:5—Sequence of promoter region of a VH4 allele.

SEQ ID NO:6—Sequence of promoter region of a VH4 allele.

SEQ ID NO:7—Consensus sequence of SEQ ID NO's 2 to 6.

SEQ ID NO:8—Plasmid pME18SasFP499.

SEQ ID NO:9—Sequence of enhancer of human immunoglobulin D segment locus.

SEQ ID NO:10—Sequence of enhancer of human immunoglobulin heavy locus on chromosome 14.

SEQ ID NO:11—Sequence of sheep immunoglobulin heavy chain 5′ intronic enhancer.

SEQ ID NO:12—Sequence of mouse 3′ IgH regulatory enhancer.

SEQ ID NO:13—Sequence of murine IgH enhancer.

SEQ ID NO:14—Sequence of mouse 3′ kappa enhancer.

SEQ ID NO:15—Promoter sequence of mouse immunoglobulin VH gene.

SEQ ID NO:16—Promoter sequence of mouse immunoglobulin V1 gene.

SEQ ID NO:17—Promoter sequence of mouse immunoglobulin mu heavy chain gene.

SEQ ID NO:18—Promoter sequence of mouse immunoglobulin VH gene.

SEQ ID NO:19—Homo sapiens germline IgH chain (ProV4-39) gene fragment.

SEQ ID NO:20—Homo sapiens germline IgH chain (ProV3-30) gene fragment.

SEQ ID NO:21—Homo sapiens germline IgH chain (ProV3-9) gene fragment.

SEQ ID NO:22—Homo sapiens germline IgH chain (ProV1-18) gene fragment.

SEQ ID NO:23—GPI signal from human decay-accelerating factor.

SEQ ID NO:24—Polynucleotide encoding SEQ ID NO:23.

SEQ ID NO:25—GPI signal from porcine membrane dipeptidase.

SEQ ID NO:26—Polynucleotide encoding SEQ ID NO:25.

SEQ ID NO:27—GPI signal from rat ceruloplasmin.

SEQ ID NO:28—Polynucleotide encoding SEQ ID. NO:27.

SEQ ID NO:29—GPI signal from mouse Thy-1.

SEQ ID NO:30—Polynucleotide encoding SEQ ID NO:29.

SEQ ID NO:31—Transmembrane domain of murine B7-1.

SEQ ID NO:32—Polynucleotide encoding SEQ ID NO:31.

SEQ ID NO:33—Signal sequence of CD59.

SEQ ID NO:34—Polynucleotide encoding SEQ ID NO:33.

SEQ ID NO's 35 to 86, 88 to 90, 92 to 103, 105, 106, 108 to 120, 124 to 129 Oligonucleotide primers.

SEQ ID NO:87—Plasmid 3 kb15a-7-4T.

SEQ ID NO:91—Plasmid KW2.

SEQ ID NO:104—Plasmid pME18sCD26asFP499.

SEQ ID NO:107—Sequence of coding region of AICDA cDNA.

SEQ ID NO:110—Plasmid KW3.

SEQ ID NO:121—Plasmid 3 kb15a-7-4TΔMluI-NaeI.

SEQ ID NO:122—Region of Plasmid 3 kb15a-7-4TΔMluI-NaeI that is integrated as described in Example 13.

SEQ ID NO:123—Parental 3 kb15a-7-4TΔMlu I-Nae I reference sequence—see Example 13.

SEQ ID NO:130—Plasmid pME18sEGFPCD26.

### DETAILED DESCRIPTION OF THE INVENTION

General Techniques

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Animal Cell Culture: Practical Approach, Third Edition (John R. W. Masters, ed., 2000), ISBN 0199637970, whole of text;
6. Immobilized Cells and Enzymes: A Practical Approach (1986) IRL Press, Oxford, whole of text;
7. Perbal, B., A Practical Guide to Molecular Cloning (1984);
8. Methods In Enzymology (S. Colowick and N. Kaplan, eds., Academic Press, Inc.), whole of series;
9. J. F. Ramalho Ortigão, “The Chemistry of Peptide Synthesis” In: Knowledge database of Access to Virtual Laboratory website (Interactiva, Germany);
10. Sakakibara, D., Teichman, J., Lien, E. L. and Fenichel, R. L. (1976). Biochem. Biophys. Res. Commun. 73 336-342
11. Merrifield, R. B. (1963). J. Am. Chem. Soc. 85, 2149-2154.
12. Barany, G. and Merrifield, R. B. (1979) in The Peptides (Gross, E.; and Meienhofer, J. eds.), vol. 2, pp. 1-284, Academic Press, New York.
13. Wüinsch, E., ed. (1974) Synthese von Peptiden in Houben-Weyls Metoden der Organischen Chemie (Muler, E., ed.), vol. 15, 4th edn., Parts 1 and 2, Thieme, Stuttgart.
14. Bodanszky, M. (1984) Principles of Peptide Synthesis, Springer-Verlag, Heidelberg.
15. Bodanszky, M. & Bodanszky, A. (1984) The Practice of Peptide Synthesis, Springer-Verlag, Heidelberg.
16. Bodanszky, M. (1985) Int. J. Peptide Protein Res. 25, 449-474.
   Hypermutating Cells

In the context of the present invention, the hypermutating cells may be bacterial, yeast, avian, fungal, insect or mammalian cells. Examples of suitable hypermutating cells are described below.

Bacterial Hypermutating Strains:

(i) Epicurian coli mutator strain XL1-Red (triple DNA repair deficient-mutD, mutS, mut T) by Stratagene;
(ii) Escherichia coli mutator strain MutD5 (MutD5-FIT, mutated DNAQ gene) Irving R A, Kortt A A, Hudson P J (1996). Immunotechnology 2(2) 127-43;
(iii) Escherichia coli strain FC40. Foster P L (2000) Bioessays 22(12): 1067-74 and Powell S C & Wartwell R M (2001) Mutation Research 473 (2) 219-28;
(iv) Serogroup B meningococcal strains BF18, BF21 (defect in methyl-directed mismatch repair—lack DNA adenine methyltransferase (Dam) activity). Bucci et al. (1999) Mol Cell 3 (4) 435-45.
   Yeast Hypermutating Strains:
(i) Saccharomyces cerevisiae strain (mlh1 Δ mutant). Shcherbakova & Kunkel (1999). Molecular and Cellular Biology 19(4) 3177-3183.
(ii) Saccharomyces cerevisiae strain DAG60 (msh2 mutant). (deficient in mismatch repair system). Drotschmann et al. (1999) Proc. Natl Acad Sci USA 96 2970-2975.
   Mammalian Hypermutating Strains:
(i) Human B cell lines from Burkitt's lymphoma strains: RAMOS, BL2, BL41, BL70, Nalm-6. Sale & Neuberger (1998) Immunity 9 (6) 859-869.
(ii) BL2: Denepouxs et al. (1997) Immunity 6(1) 35-46 and Poltorasky et al. (2001) Proc Natl Acad Sci USA 98 (14) 7976-81
(iii) Human pre B cell line strain 18.81. Bachl et al. (2001) Journal Immunology 166 (8) 5051-7.
   Intronic Enhancers

Examples of suitable exogenous intronic enhancers for use in the present invention include the following:

1) 1) DNA sequence of the human immunoglobulin D segment locus (Rabbitts et al (1983) Nature 306 (5945), 806-809):

| | |
|---|---|
| 5′ CGGCCCCGATGCGGGACTGCGTTTTGACCATCATAAATCAAGTTTATTTT | (SEQ ID NO:9) |
| TTTAATTAATTGAGCGAAGCTGGAAGCAGATGATGAATTAGAGTCAAGATG | |
| GCTGCATGGGGGTCTCCGGCACCCACAGCAGGTGGCAGGAAGCAGGTCAC | |
| CGCGAGAGTCTATTTTAGGAAGCAAAAAAACACAATTGGTAAATTTATCAC | |
| TTCTGGTTGTGAAGAGGTGGTTTTGCCCAGGCCCAGATCTGAAAGTGCTCT | |
| ACTGAGCAAAACAACACCTGGACAATTTGCGTTTCTAAAATAAGGCGAGGC | |
| TGACCGAAACTGAAAAGGCTTTTTTTAACTATCTGATTTCATTTCCAATCT | |
| TAGCTTATCAACTGCTAGTTTGTGCAAACAGCATATCAACTTCTAAACTGCA | |
| TTCATTTTTAAAGTAAGATGTTTAAGAAATTAAACAGTCTTAGGGAGAGTTT | |
| ATGACTGTATTCAAAAAGTTTTTTAAATTAGCTTGTTATCCCTTCATGTGTA | |
| ATTAATCTCAAATACTTTTTCGATACCTCAGAGCATTATTTTCATAATGACT | |
| GTGTTCACAATCTTTTT 3′ | |

2) Homo sapiens immunoglobulin heavy locus (IGH.1@) on chromosome 14 (Ravetch et al (1981) Cell 27(3 Pt 2), 583-591):

| | |
|---|---|
| 5′ GGCCCCGATGCGGGACTGCGTTTTGACCATCATAAATCAAGTTTATTTTTT | (SEQ ID NO:10) |
| TAATTAATTGAGCGAAGCTGGAAGCAGATGATGAATTAGAGTCAAGATGG | |
| CTGCATGGGGGTCTCCGGCACCCACAGCAGGTGGCAGGAAGCAGGTCACC | |
| GCGAGAGTCTATTTTAGGAAGCAAAAAAACACAATTGGTAAATTTATCACT | |
| TCTGGTTGTGAAGAGGTGGTTTTGCCCAGGCCCAGATCTGAAAGTGCTCTA | |
| CTGAGCAAAACAACACCTGGACAATTTGCGTTTCTAAAATAAGGCGAGGCT | |
| GACCGAAACTGAAAAGGCTTTTTTAACTATCTGAATTTCATTTCCAATCTT | |
| AGCTTATCAACTGCTAGTTTGTGCAAACAGCATATCAACTTCTAAACTGCAT | |
| TCATTTTTAAAGTAAGATGTTTAAGAAATTAAACAGTCTTAGGGAGAGTTT | |
| ATGACTGTATTCAAAAAGTTTTTTAAATTAGCTTGTTATCCCTTCATGTGAT | |
| AATTAATCTCAAATACTTTTTCGATACCTCAGAGCATTATTTTCATAATGAC | |
| TGTGTTCACAATCTTTTT 3′ | |

3) Ovis aries immunoglobulin heavy chain 5′ intronic enhancer (Dufour et al (1996) J. Immunol 156:2163-2170):

| | |
|---|---|
| 5′ CTGCGAATACCGAGACGGGGCCTCTCAAAGCCACCCCTGATAGTCTGGAA | (SEQ ID NO:11) |
| AATTGAAACTTTAAAAAGAGAGATGTTTAAAGTATTTTAAATTTTTATCATT | |
| TAATTAACAACTGCGAATCATGGCTTTGGAGAGTTGAGTAAGAGTTTGGCT | |
| GAAAAGTACTAACTAGGTTCCATCGGCCCTCGGCCCCAATTCAGGGCTGTT | |
| TTGAGAATAATAAATTCAGCTTATTTTTTTAATGTAATTGGTGGTGCCGAGT | |
| TAGTCAAGATGGCCACGGGCCAGACTGACCACCTGCAGCAGGTGGCAGGA | |
| AGCATGTCCACTTGAGAGTCTGTTTTTGGAAGCAAGAAAAAACAGTTGGTA | |
| AATTTATCGCTTCTGGTTTCCAAAAGGTGGTTTGCGGCTGGTTTTGCCCAGC | |
| CCCACAGAACCGAAAGTGTTCCACTGAGCACAACAGCACCTGGCTAATTTG | |
| CATTTCTAAAATAAGGCGCAGATGCTGACCGAAACTGGAAGGTTCCTCTTC | |
| TAACTATTTGAGTTAACTTCAGCTTTAGCTTATCAACTGCTCACTTATCTTCA | |
| TTTTCAAAGTCGATGTTTAAGAAAGCCACCTGTCTCGGGTGCACTGTCTCGG | |
| TGCATTGCTGCACTCTCTGATGAGCCGTCCTTCAAGGTGGTTGAGCTGAG 3′ | |

4) Mouse 3′ IgH regulatory enhancers (C alpha3′E and hs3) (Saleque et al (1997) J. Immunol. 158 (10), 4780-4787):

| | |
|---|---|
| 5′ CTAGATACTGAGTTCTGGTTCTAATAACTGGCTCCTGTACTGATGGATGG | (SEQ ID NO:12) |
| GTCCTGACTAGTCATTGGGCCCTGATCCTCAACATTGACTTCAAAACCTGAA | |
| CTCTAGCCCCATGCCTCATTCACATTAGGATGATCCCTACAGGGGATTCCTG | |
| CAGAAGATTCCAGAATCCCCACAACACTGTTCACACACTGGGCTGCAACTG | |
| GGACAGTGACCCTTTTGACTCATAGGACTTGCCAGGCACAGAGGCACAGAA | |
| TGGAGACAAAGCAAGCCCAGGACCCTGGAGATGGAGCCTCTGGTGGGGTC | |
| TACAGATGTGGGGTCAGCATCGTAGGGAGGTTTGCAGGGCAGGTGTGGGG | |
| CAGGGCAGAGGTAGTCATGCTTATAGATACTATTTTTCTCTCCTCTGGAGCC | |
| TCCTTTGTCTATCACCTGCTGTCCTGGGATCTCTATCTGGGGTCAACAATGT | |
| TTGCAGTACAGGTGTGGGGGTAGGGCAGGGATGCTCACATTAGCAACTTGT | |
| TTTTCTCTCTTCTGAAGTCTCTGTTGTCTATCACCTGCTGAAACATTCAAAGC | |
| AGCTCTCAGCTGAGGGCAGCTGAGTCATCCTGAGCCTGTCTCAGCACAGGT | |
| GCCCCAAACCAGAGCTACTGTTCTGAGAATCACATCACACTGGACCAGGCC | |
| AGGTGGGCCTGGGACATGGATGAGGGGTGGGAGCCAGGGGAGCCTGCCAG | |
| GGGCTGAGGAGGCCCCAACCCCCACTACCCAAGGCCATCCACACCTGTGCC | |
| TTAGTGAGGCCATGTTCTGTCCCAATGAGAACAAGTCCAATTAAGATTAAG | |
| TATGGTCTTCCCAGGACTATCCAGAGCTAAGGGGTGTCAGCCAGGGACAAC | |
| CCAGACCAGCCTGAGGTCAGCCAGCATCACCCAAGGCCACACAGCTATTCT | |
| GGCTAGAGGACTAGATAGCTAGCTCATCGAGGCCCTGGAGATGCAGAATG | |
| GAAGAGTTTATCCCTGCCAGACAGGGCTCATCAGAAAGGCAGGTATCTCAC | |
| TACACATGACCTCCCTGAATATTTCCCAGAGTCCAGTTGGTTCTAG 3′ | |

5) Murine IgH enhancer (Kadesch et al (1986) Nucleic Acids Res. 14 (20), 8209-8221):

| | |
|---|---|
| 5′ AGTCAAGATGGCCGATCAGAACCAGAACACCTGCAGCAGCTGGCAGGAA | (SEQ ID NO:13) |
| GCAGGTCATGTGGCAAGGCTATTTGGGGAAGGGAAAATAAAACCACTAGG | |
| TAAACTTGTAGCTGTGGTTTGAAGAAGTGGTTTTGAAACACTCTGTCCAGCC | |
| CCACCAAACCGAAAGTCCAGGCTGAGCAAAACACCACCTGGGTAATTTG | |
| CATTTCTAAA ATAAGTTGAGG 3′ | |

3′ Kappa Enhancers

An example of a suitable exogenous 3′ kappa enhancer for use in the present invention is as follows.

1) Murine 3′ kappa enhancer (Meyer and Neuberger (1989) EMBO J. 8 (7), 1959-1964):

| | |
|---|---|
| 5′ AGCTCAAACCAGCTTAGGCTACACAGAGAAACTATCTAAAAAATAATTA | (SEQ ID NO:14) |
| CTAACTACTTAATAGGAGATTGGATGTTAAGATCTGGTCACTAAGAGGCAG | |
| AATTGAGATTCGAACCAGTATTTTCTACCTGGTATGTTTTAAATTGCAGTAA | |
| GGATCTAAGTGTAGATATATAATAATAAGATTCTATTGATCTCTGCAACAA | |
| CAGAGAGTGTTAGATTTGTTTGGAAAAAAATATTATCAGCCAACATCTTCT | |
| ACCATTTCAGTATAGCACAGAGTACCCACCCATATCTCCCCACCCATCCCCC | |
| ATACCAGACTGGTTATGATTTTCATGGTGACTGGCCTGAGAAGATTAAAA | |
| AAAGTAATGCTACCTTATTGGGAGTGTCCCATGGACCAAGATAGCAACTGT | |
| CATAGCTACCGTCACACTGCTTTGATCAAGAAGACCCTTTGAGGAACTGAA | |
| AACAGAACCTTAGGCACATCTGTTGCTTTCGCTCCCATCCTCCTCCAACAGC | |
| CTGGGTGGTGCACTCCACACCCTTTCAAGTTTCCAAAGCCTCATACACCTGC | |
| TCCCTACCCCAGCACCTGGCCAAGGCTGTATCCAGCACTGGGATGAAAATG | |
| ATACCCCACCTCCATCTTGTTTGATATTACTCTATCTCAAGCCCCAGGTTAG | |
| TCCCCAGTCCCAATGCTTTTGCACAGTCAAAACTCAACTTGGAATAATCAGT | |
| ATCCTTGAAGAGTTCTGATATGGTCACTGGGCCCATATACCATGTAAGACA | |
| TGTGGAAAAGATGTTTCATGGGGCCCAGACACGTTCTAG 3′ | |

Promoter Sequences

Examples of suitable exogenous promoter sequences for use in the present invention include:

1) (Kataoka et al (1982). J Biol. Chem. 257: 2777-285):

(SEQ ID NO:15)

5′ AAGCAGCCCTCAGGCAGAGGATAAAAGCTCACACTAACTGAGAAGC
TCCATCCTCTTCTC 3′

2) (Clarke et al (1982). Nucleic Acids Res. 10: 7731):

(SEQ ID NO:16)

5′ AATTAGGCCACCCTCATCACATGAAAACCAGCCCAGAGTGACTCTAG
CAGTGGGATCCTG 3′

3) Grosschedl and Baltimore D., (1985). Cell 41: 885-897):

(SEQ ID NO:17)

5′ CATGTGCGACTGTGATGATTAATATAGGGATATCCACACCAAACATC
ATATGAGCCCTAT 3′

4) (Schiff et al (1986). J. Exp. Med. 163: 573-587):

(SEQ ID NO:18)

5′AACATGAGTCTGTGATTATAAATACAGAGATATCCATACCAAACAAC
TTATGAGCACTGT 3′

Murine B lymphocyte VH promoters (e.g. V1 Ig VH promoter, BCL1 VH promoter) may also be used in the methods of the present invention.

Human Immunoglobulin Heavy Chain Promoters

The following human heavy chain promoters are described in in Haino et al (1994) J. Biol. Chem. 269 (4), 2619-2626:

1) Homo sapiens germline IGH chain (ProV4-39) gene fragment:

| | |
|---|---|
| 5′ ATCCCAAAATCTGTCNTTGATCCAGGATCACACTCATCTCTCAGACCAGC | (SEQ ID NO:19) |
| TCCTTCAGCACATCTCTTTACCTGGAAGAAGAGGACTCTGGGCTTGGAGAG | |
| GGGAGCCCCCAAGAAGAGAACTGAGTTCTCAAAGGGCACAGCCAGCATTC | |
| TCCTCCCAGGGTGAGCTCAAAAGACTGGCGCCTCTCTCATCCCTTTTCACTG | |
| CTCCGTACAAACGCACCACCCCCATGCAAATCCTCACTTAGGCGCCCACAG | |
| GAAGCCACCACACATTTCCTTAAATTCAGGTCCAACTCATAAGGGAAATGC | |
| TTTCTGAGAGTCATGGATCTCATGTGCAAGAAA 3′ | |

2) Homo sapiens germline IGH chain (ProV3-30) gene fragment:

| | |
|---|---|
| 5′ AGATATAACTATATTTTCCTGAATGATGGAATTACTACCAGTCTCCCCCA | (SEQ ID NO:20) |
| GGACACTTCATCTGCCCTGAGCCCAGCCTCTCCTCAGATGTCCCACCCAGA | |
| GCTTGCTATATAGTGGGGGACATGCAAATAGGGCCCTCCCTCTACTGATGA | |
| AAACCAGCCCAGCCCTGACCCTGCAGCTCTGGGAGAGGAGCCCAGCACTA | |
| GAAGTCGGCGGTGTTTCCATTCGGTGATCAGCACTGAACACAGAGGAC | |
| TCACC 3′ | |

3) Homo sapiens germline IGH chain (ProV3-9) gene fragment:

| | |
|---|---|
| 5′ CAGTAGAAATGCTAATAAGAATTAATTGTTTATGAAGTGTAATCACTCTG | (SEQ ID NO:21) |
| GGACACAGCCCACTCAGAGGCATCCCTTCCAGAACCCGCTATATAGTAGGA | |
| GACATGCAAATAGGGCCCTCCCTCTGCTGATGAAAACCAGCCCAGCCCTGA | |
| CCCTGCAGCTCTGGGAGAGGAGCCCCAGCCCTGAGATTCCCAGGTGTT | |
| TCCATTCAGTGATCAGCACTGAACACAGAGGACTCACC 3′ | |

4) Homo sapiens germline IGH chain (ProV1-18) gene fragment:

| | |
|---|---|
| 5′ GATGGGTAGGGGATGCGTGTCCTCTAACAGGATTACGTCTTGAACCCTCA | (SEQ ID NO:22) |
| GCTTCTACAATTGTGTCGTCCATGTGTCATGTATTTGCTCTTTCTCATCCTGG | |
| GTCAGGAATTGGGCTATTAAATAGCATCCTTCATGAATATGCAAATAACTG | |
| AGGTGAATATAGATATCTGTGTGCCCTGAGAGCATCACCCAAAAACCACAC | |
| CCCTCCTTGGGAGAATCCCCTAGATCACAGCTCCTCACC 3′ | |

Methods for Selection of Nucleic Acids or Proteins/Peptides with an Altered Phenotype

The terms “altered phenotype”, “desired activity” and “altered activity” are generally used interchangeably herein.

In particular embodiments of the present invention, the mutated nucleic acid, or gene product encoded thereby, is subjected to an assay for identifying an altered phenotype. Suitable procedures for identifying altered phenotypes include, but are not limited to, those described below.

Protein/Peptide Display and Selection

In one embodiment of the invention, proteins encoded by nucleic acids obtained using the methods of the invention are displayed on the surface of the hypermutating cells.

One well-known peptide display method involves the presentation of a peptide sequence on the surface of a filamentous bacteriophage, typically as a fusion with a bacteriophage coat protein. The bacteriophage library can be incubated with an immobilized, predetermined macromolecule or small molecule (e.g., a receptor) so that bacteriophage particles which present a peptide sequence that binds to the immobilized macromolecule can be differentially partitioned from those that do not present peptide sequences that bind to the predetermined macromolecule. The bacteriophage particles (i.e., library members) which are bound to the immobilized macromolecule are then recovered and replicated to amplify the selected bacteriophage subpopulation for a subsequent round of affinity enrichment and phage replication. After several rounds of affinity enrichment and phage replication, the bacteriophage library members that are thus selected are isolated and the nucleotide sequence encoding the displayed peptide sequence is determined, thereby identifying the sequence(s) of peptides that bind to the predetermined macromolecule (e.g., receptor). Such methods are further described in WO 91/17271, WO 91/18980, WO 91/19818 and WO 93/08278.

WO 93/08278 describes a recombinant DNA method for the display of peptide ligands that involves the production of a library of fusion proteins with each fusion protein composed of a first polypeptide portion, typically comprising a variable sequence, that is available for potential binding to a predetermined macromolecule, and a second polypeptide portion that binds to DNA, such as the DNA vector encoding the individual fusion protein. When transformed host cells are cultured under conditions that allow for expression of the fusion protein, the fusion protein binds to the DNA vector encoding it. Upon lysis of the host cell, the fusion protein/vector DNA complexes can be screened against a predetermined macromolecule in much the same way as bacteriophage particles are screened in the phage-based display system, with the replication and sequencing of the DNA vectors in the selected fusion protein/vector DNA complexes serving as the basis for identification of the selected library peptide sequence(s).

The displayed protein/peptide sequences can be of varying lengths, typically from 3-5000 amino acids long or longer, frequently from 5-100 amino acids long, and often from about 8-15 amino acids long. A library can comprise library members having varying lengths of displayed peptide sequence, or may comprise library members having a fixed length of displayed peptide sequence. Portions or all of the displayed peptide sequence(s) can be random, pseudorandom, defined set kernal, fixed, or the like. The display methods include methods for display of single-chain antibodies, such as nascent scFv on polysomes or scFv displayed on phage, which enable large-scale screening of scFv libraries having broad diversity of variable region sequences and binding specificities.

Another method of display is bacterial surface display. The protein of interest is expressed on the bacterial cell surface as a fusion with one of the following proteins, OmpA, LamB, PhoE, FliC, PALD, and EaeA intimin. Alternatively it may be expressed in the periplasm (periplasmic expression with cytometric screening, (PECS)) (Chent et al., 2001). Whilst experiments demonstrate expression on the bacterial cell surface, this display system is not operating as well or used as frequently as the yeast surface display. The nature of a prokaryotic system itself may explain why the system is not always preferred. Firstly, bacteria lack the protein folding and post-translational modification machinery required for the presentation of an eukaryotic protein. Secondly, the protein of interest needs to be expressed in a soluble form. Thirdly, steric interference from the bacterial lipopolysaccharide layer can potentially impede binding to larger macromolecular antigens. Despite being a single cell system (one plasmid to each bacterium), capable of displaying thousands of copies of the protein of interest on the cell surface and being amenable to screening using flow cytometry, this system offers no additional advantages over the yeast surface display system.

A further method of display, is yeast surface display. The protein of interest is fused to an alpha agglutinin subunit called Aga2p and expressed on the surface of the yeast cell. This form of display appears to be very successful and offers several advantages over other display systems. These include; correct protein folding and secretion (homologous to that in mammalian cells), display of large numbers of protein on yeast cell surface, single cell system (i.e. each yeast cell contains only one plasmid copy), system is amenable to screening using flow cytometry which offers finer quantitative discrimination between mutants and the dissociation constant (K_{D}) can be estimated in situ in the display format without having to subclone. The major disadvantage of this system is that the library size is restricted due to low transfection efficiency. To date no one has managed to overcome this limitation of the system.

Proteins can be anchored to the surface of mammalian cells via a number of different anchors including, but not limited to, Type I transmembrane domains (TM I), type II transmembrane (TM II) and glycosylphosphatydlinisitol (GPI).

Examples of suitable GPI anchor attachment signals include:

(i) GPI signal from human decay-accelerating factor (DAF). Caras I W, Weddell G N, Davits M A, Nussenzweig W, Martin D W. (1987) Science 238(4831):1280-3 Accession No.: AY055758

| | |
|---|---|
| Peptide sequence: | |
| PNKGSTTSGTTRLLSGHTCFTLTGLLGTLVTMGLLT | (SEQ ID NO:23) |
| Nucleotide sequence: 1158-1268 | |
| CCAAATAAAGGAAGTGGAACCACTTCAGGTACTACCCGTCTTCTATCTGGG | (SEQ ID NO:24) |
| CACACGTGTTTCACGTTGACAGGTTTGCTTGGGACGCTAGTAACCATGGGC | |
| TTGCTGACT | |

(ii) GPI signal from porcine membrane dipeptidase. Hooper N M, Low M G, Turner A J. (1987) Biochem. J. 244(2):465-9 Accession No.: E04233

| | |
|---|---|
| Peptide sequence: | |
| CRTNYGYSAAPSLHLPPGSLLASLVPLLLLSLP | (SEQ ID NO:25) |
| Nucleotide sequence: 1248-1346 | |
| TGCCGGACGAATTACGGCTACTCAGCCGCCCCCAGCCTCCACCTCCCGCCG | (SEQ ID NO:26) |
| GGCTCGCTGCTGGCCTCCCTCGTGCCCCTCCTCCTCCTCAGTCTTCCG | |

(iii) GPI signal from rat ceruloplasmin. Patel B N, Dunn R J & David S. (2000) J. Biol. Chem. 275(6):4305-10 Accession No.: AF202115

| | |
|---|---|
| Peptide sequence: | |
| ASSQSYRMTWNILYTLLISMTTLFQISTKE | (SEQ ID NO:27) |
| Nucleotide sequence: 3161-3252 | |
| GCATCGTCTCAGAGCTACAGGATGACCTGGAACATACTCTATACACTGTTA | (SEQ ID NO:28) |
| ATCAGCATGACTACTTTATTCCAAATATCTACCAAGGAG | |

(iv) GPI signal from mouse Thy-1. Bernasconi E, Fasel N & Wittek R. (1996) J. Cell Sci. 109(6):1195-201

| | |
|---|---|
| Peptide sequence: | |
| SSNKSISVYRDKLVKCGGISLLVQNTSWMLLLLLSLSLLQALDFISL | (SEQ ID NO:29) |
| Nucleotide sequence: | |
| AGCTCCAATAAAAGTATCAGTGTGTATAGAGACAAGCTGGTCAAGTGTGGC | (SEQ ID NO:30) |
| GGCATAAGCCTGCTGGTTCAGAACACATCCTGGATGCTGCTGCTGCTGCTTT | |
| CCCTCTCCCTCCTCCAAGCCCTGGACTTCATT | |

(v) Dictyostelium discoideum protein 1I. Stevens B A, White I J, Hames B D Hooper N M. (2001) Biochimica et Biophysica Acta 1511: 317-329.

(vi) Plasmodium falciparum merozoite surface protein-1. Burghas P A, Gerold P, Pan W, Schwartz R T, Lingelbach K, Burjard H. (1999) Molecular and Biochemical Parisitology 104:171-183.

An example of a suitable transmembrane domain for use as an anchor domain in the present invention is:

(i) The transmembrane domain of murine B7-1. Chou W., Liao K., Jiang S. Y., Yeh M. Y. and Roffler S. R. (1999) Biotechnol Bioeng. 1999 Oct. 20;65(2):160-9. Accession No.: AH00465S3

| | |
|---|---|
| Peptide sequence: | |
| PEDPPDSKNTLVLFGAGFGAVITVVVIVVIIKCFCKH | (SEQ ID NO:31) |
| Nucleotide sequence: 171-281: | |
| CCCAGAAGACCCTCCTGATAGCAAGAACACACTTGTGCTCTTTGGGGCAGG | (SEQ ID NO:32) |
| ATTCGGCGCAGTAATAACAGTCGTCGTCATCGTTGTCATCATCAAATGCTTC | |
| TGTAAGCAC | |

Other suitable examples include those provided in Table 1.

TABLE 1
Examples of anchors suitable for surface display:

| | Molecule | |
|---|---|---|
| Anchor type | name | Accession No. |
| Transmembrane type I | CD1a | X04450 (gi 32495) |
| (TM I) | CD68 | BC05557 (gi 33869409) |
| Transmembrane type II | CD10 | Y00811 (gi 29625) |
| (TM II) | CD13 | X13276 (gi 28677) |
| | CD26 | M74777 (gi 180082) |
| Glycosylphosphatydlinisitol | CD14 | X113334 (gi 29740) |
| (GPI) | CD24 | M58664 (gi 180167) |
| | CD48 | M59904 (gi 180138) |
| | CDw52 | X62466 (gi 29645) |
| | CD55 | M31516 (gi 181467) |
| | CD59 | X16447 (gi 29805) |
| | CD67 | X52378 (gi 29918) |

Signal Peptide Sequences

For TM1 and GPI anchors, an N-terminal signal sequence as well as a C-terminal signal sequence is preferably be added to the polypeptide if it is not normally found on the cell surface. For TM2 the signal and anchor sequence are one and the same and are added to the N-terminus of the polypeptide. To achieve adequate levels of surface expression, it may be necessary to mutate the initiation codon of the target gene so that only chimeric proteins consisting of the signal and/or anchor fused to the target protein are produced.

An example of an appropriate signal sequence is the signal sequence of CD59: Accession No.: X16447 (gi 180082)

| | |
|---|---|
| Peptide sequence: | |
| MGIQGGSVLFGLLLVLAVFCHSGHS | (SEQ ID NO:33) |
| Nucledtide sequence: 64-138 | |
| 5′ ATGGGAATCCAAGGAGGGTCTGTCCTGTTCGGGCTGCTGCTCGTCCTGGC | (SEQ ID NO:34) |
| TGTCTTCTGCCATTCAGGTCATAGC 3′ | |

Proteins/peptides encoded by mutant nucleic acids obtained using the methods of the invention can be used in a, number of yeast based methods to detect protein-protein interactions. One well known system is the yeast two-hybrid system (Fields and Song 1989) which has been used to identify interacting proteins and to isolate the corresponding encoding genes. In this system, prototrophic selectable markers which allow positive growth selection are used as reporter genes to facilitate identification of protein-protein interactions. Related systems which may be employed include the yeast three-hybrid system (Licitra and Liu 1996) and the yeast reverse two-hybrid system (Vidal et al. 1996). Such procedures are known to those skilled in the art.

### EXAMPLES

### Example 1

### Defining a region in RAMOS Cells for Integration of Target Genes

Methods & Materials

Cell Line and Cell Culture Conditions

The RAMOS strain RA 1 was obtained from the American Tissue Culture Collection (ATCC-CLR-1596). This strain is IgM positive and expresses the interleukin 4 (IL-4) and CD23 receptors. Cells were maintained in RPMI 1640 medium (Gibco BRL) supplemented with 10% heat inactivated fetal calf serum (FCS) and penicillin (100 U/ml) and streptomycin (100 μg/ml), and incubated at 37° C. with 5% CO₂.

Extraction of DNA from Cells

Cells were harvested and centrifuged at 1500 rpm and resuspended in PBS. DNA was extracted from cells using a Genoprep DNA isolation kit (Scientifix, Australia) according to manufacturer's instructions. Briefly, after removing the supernatant 375 μl of lysis and binding solution was added to cells (5×10⁵), together with 20 μl Genoprep DNA magnetic beads. This mixture was vortexed for five seconds then incubated at room temperature for a minimum of ten minutes on a rocker, or rotating wheel. Beads with attached DNA were collected using a magnet, the supernatant was removed and 450 μl of washing solution was added. The mixture was subsequently vortexed for five seconds and the beads were collected as described previously. This washing procedure was repeated twice, with the final wash solution being 70% ethanol. Beads were resuspended in 450 μl of 70% ethanol and transferred to a new tube. After removing the supernatant, 450 μl of sterile water was added to the beads and removed immediately. The beads were then resuspended in 200 μl of sterile water and incubated at 70° C. for two minutes to elute the DNA. The beads were collected again using a magnet and the supernatant containing the eluted DNA was transferred to a new tube.

The quantity and quality of isolated DNA was determined by spectrometry and electrophoresis. A culture containing 5×10⁵ cells consistently yielded 10 ng/μl of DNA. This DNA migrated as a single band at approximately 23 kbp on a 0.9% gel indicating that the genomic DNA was intact.

Sequencing of the 5′ Region Upstream of the Rearranged VH Allele

The homologous sequence upstream of the site of integration chosen for the vector corresponds to a ˜5 kb fragment between the VH₇₋₃₅ and VH₄₋₃₄ alleles of the immunoglobulin heavy chain locus of the RAMOS cell line (corresponding to the sequence gi 4512287 nucleotides 54521-59517).

RAMOS RA-1 genomic DNA was prepared using the Genoprep DNA isolation kit (Scientifix, Australia). Platinum PfX DNA polymerase (GibcoBRL, Life Technologies) was used to amplify fragments varying from 300 bp to 1000 bp from genomic DNA. The reaction included 1×PfX amplification buffer, 50 mM Magnesium sulfate, 1×PCR enhancer solution, forward primer (10 pM), reverse primer (10 pM), dNTPs (10 mM each) template DNA (100 ng), platinum PfX DNA polymerase (0.6 U) and sterile water in a final volume of 20 μl. Cycling conditions were as follows; one cycle of 95° C. for 5 minutes, 30 cycles of 95° C. for seconds, 60° C. for 30 seconds, 68° C. for one and a half seconds, and one cycle of 72° C. for 7 minutes. Annealing temperatures and extension times for some primer sets varied, ranging from 55° C. to 65° C. and one minute to two and a half minutes respectively. Primers were designed based on the human germline DNA for immunoglobulin heavy-chain variable region, complete sequence gi 4512287 nucleotides 54786 to 59721 (Table 2). A second PCR reaction was performed using 0.5/20 μl of the first PCR as a template to gain sufficient DNA for cloning.

PCR products were run on 1.0% agarose gels and DNA was extracted using Nucleospin Extraction Kit (Nagel-Macherey, Germany) according to manufacturer's instructions. The purified DNA was digested with restriction enzymes EcoR I and Hind III. Digested products were cleaned up and concentrated using phenol extraction followed by ethanol precipitation. These products were ligated into into pBluescript SK+ (Stratagene, Texas, USA) and transformed into Escherichia coli XL1 Blue electro-competent bacteria. Minipreps were prepared from 5 ml overnight cultures using QIAprep miniprep spin kit (Qiagen, Calif., USA) and sequenced using an ABI 373 DNA sequencer with primers T3 and T7.

Sequences were analysed using BLAST program (NBCI, http://www.ncbi.nlm.nih.gov/BLAST/) and assembled in Clone Manager Suite 7 (Scientific and Educational Software). The assembled sequence is set out as nucleotides 1 to 5190 of SEQ ID NO:1. This sequence shares 99% similarity with the published sequence gi 4512287 nucleotides 54521-59517.

Cloning of the 5 kb Fragment Upstream of the Rearranged VH Allele from Genomic DNA

Genomic DNA was prepared using Genoprep DNA isolation kit (Scientifix, Australia). Platinum PfX DNA polymerase (Invitrogen, Calif. USA ) was used to amplify the 5 kb fragment from genomic DNA. The reaction included 1×PfX amplification buffer, 50 mM magnesium sulfate, 1×PCR enhancer solution, forward primer 8771 (5′CCATCGATAATTTAGTTTTCACGGGGCATCTGCAGGGT 3′) 10 pM, reverse primer 8872 (5′GGGGTACCGTTCTTGTGCAGGAGGTCCATGACTCTCAG 3′) 10 pM, dNTPs (10 mM each) template DNA (100 ng), platinum PfX DNA polymerase (0.6 U) and sterile water in a final volume of 20 μl. Cycling conditions were as follows; one cycle of 94° C. for 15 seconds, fifteen cycles of 94° C. for 10 seconds, 68° C. for 3.5 minutes, 15 cycles of 94° C. for 10 seconds, 68° C. for 3.5 minutes with and an extra 15 seconds added each cycle, and one cycle of 72° C. for 7 minutes. A second PCR reaction was performed using 0.5/20.0 μl of the first PCR reaction as a template to gain sufficient DNA for detection using ethidium bromide.

PCR products were run on a 0.9% agarose gel and DNA was extracted using Gel Extraction Kit (Qiagen, Calif., USA). Purified DNA was cloned into pPCRScript using the PCR-Script Amp cloning kit (Stratagene, Tex., USA) at the Srf I site. The resulting construct was referred to as 5 kb PCRScript 15a-7 (7971 bp). This construct was sequenced using primers in Table 3.

TABLE 2

Primers used for genomic PCR to sequence the 5′ region
upstream of the rearranged VH allele (VH₄₋₃₄) in RAMOS RA-1

Homologous

| | |
|---|---|
| Primer | sequence in gi |

| | | |
|---|---|---|
| Name | Sequence | AB019439 |

| | | |
|---|---|---|
| 8444 | 5′ CCGGAATTCAATTTGAGATTGTGTGTGAGATCTCAGGAG 3′ | NT 58890-58920 |
| | (SEQ ID NO:35) | |
| 8436 | 5′ CCGGAATTC ATAGACAGCGCAGGTGAGGGACAG GGTCTC 3′ | NT 59702-59731 |
| | (SEQ ID NO:36) | |
| 8438 | 5′ CCGGAATTCCTG AGA ACTCAG TTCTCTTCCTGTGGCCTC 3′ | NT 59281-59310 |
| | (SEQ ID NO:37) | |
| 8440 | 5′ CCGGAATTCAATTTGAGATTGTGTGTGAGATCTCAGGAG 3′ | NT 58890-58920 |
| | (SEQ ID NO:38) | |
| 8441 | 5′ CCCAAGCTTTCCTGTTACAACATCCATGGAGATATTTTG 3′ | NT 58420-58450 |
| | (SEQ ID NO:39) | |
| 8442 | 5′ CCGGAATTC TGAATTGCAAGAACATACCCTAGGGTGTGC 3′ | NT 58500-58530 |
| | (SEQ ID NO:40) | |
| 8464 | 5′ CCGGAATTCTAGGGCAAACAGAGGCCAGATGTTTGAGGAG 3′ | NT 57720-57750 |
| | (SEQ ID NO:41) | |
| 8466 | 5′ CCGGAATTCAATTTAACAGCATAAAAACGATCAGTCCAA 3′ | NT 57330-57360 |
| | (SEQ ID NO:42) | |
| 8470 | 5′ CCGGAATTCCGTGTTTCTGGAGCAGGGCATGGCTTTGGG 3′ | NT 56550-56580 |
| | (SEQ ID NO:43) | |
| 8472 | 5′ CCGGAATTCGTTGGGTTCCCAGTGTAGGTGATGATCCAT 3′ | NT 56160-56190 |
| | (SEQ ID NO:44) | |
| 8550 | 5′ CCGGAATTCTCCCAGGAAGTGGGTTATTTTTAAATAGTA 3′ | NT 58951-58981 |
| | (SEQ ID NO:45) | |
| 8553 | 5′ CCGGAATTCACTATAGTCACCTCAGTTAATTGCATATTC 3′ | NT 55770-55800 |
| | (SEQ ID NO:46) | |
| 8554 | 5′ CCCAAGCTTGACTTCCTTTAAAAATATCTAAAATAAGTA 3′ | NT 55300-55330 |
| | (SEQ ID NO:47) | |
| 8555 | 5′ CCGGAATTCGGTTCTCATTACAACATCCAGTTTGATAAA 3′ | NT 55380-55410 |
| | (SEQ ID NO:48) | |
| 8557 | 5′ CCGGAATTCCTCCAAGAAAAGATCTCATGCATCACCAGG 3′ | NT 54990-55020 |
| | (SEQ ID NO:49) | |
| 8558 | 5′ CCCAAGCTTAATTTAGTTTTCACGGGGCATCTGCAGGGT 3′ | NT 54520-54550 |
| | (SEQ ID NO:50) | |
| 8606 | 5′ CCCAAGCTTTGCACACCCTAGGGTATGTTCTTGCAATTC 3′ | NT 58500-58530 |
| | (SEQ ID NO:51) | |
| 8607 | 5′ CCCAAGCTTCCCAAAGCCATGCCCTGCTCCAGAAACACG 3′ | NT 56550-56580 |
| | (SEQ ID NO:52) | |
| 8608 | 5′ CCGGAATTC CCATAATATGTGAATGCGTTATTTAGG GAA 3′ | NT 55500-55529 |
| | (SEQ ID NO:53) | |
| 8687 | 5′ CCCAAGCTTCATGTTCCACGCATTACGTC 3′ | NT 54336-54355 |
| | (SEQ ID NO:54) | |
| 8689 | 5′ CCCAAGCTTAAGAGTGTTTGGGTTCACCG 3′ | NT 54927-54946 |
| | (SEQ ID NO:55) | |

TABLE 3

Primers used for sequencing clones containing the 5 kb
region upstream of the rearranged VH allele (VH₄₋₃₄)
in RAMOS RA-1

Homologous

| | |
|---|---|
| Primer | sequence in gi |

| | | |
|---|---|---|
| Name | Sequence | AB019439 |

| | | |
|---|---|---|
| 8445 | 5′ CCCAAGCTTTTAACTCAGGAGGACTCAATACACCCTGGA 3′ | NT 57640-57670 |
| | (SEQ ID NO:56) | |
| 8443 | 5′ CCCAAGCTTAAACAATACCTACAAATTCAGAAGCTCTTT 3′ | NT 58030-58060 |
| | (SEQ ID NO:57) | |
| 8471 | 5′ CCCAAGCTT AAGTCTTCTGGTTACACCTTCACCAT TAT 3′ | NT 56080-56110 |
| | (SEQ ID NO:58) | |
| 8465 | 5′ CCCAAGCTTACTCTCTTCCCTCTGTGACTAGAGCTCTGT 3′ | NT 57250-57280 |
| | (SEQ ID NO:59) | |
| 8473 | 5′ CCCAAGCTTTCAGCTTCTACAGTTGTGTCACCCATGTGT 3′ | NT 55690-55720 |
| | (SEQ ID NO:60) | |
| 8609 | 5′ CCCAAGCTTTGCAGAGTTCACTGGGTTTCCTAAAGG CAA 3′ | NT 55027-55056 |
| | (SEQ ID NO:61) | |
| 8467 | 5′ CCCAAGCTTTCACCACAAAGGAACTTTCATCTCTCCTGG 3′ | NT 56860-56890 |
| | (SEQ ID NO:62) | |
| 8439 | 5′ CCCAAGCTTTTTCACACAGAAATGTTTAGAGGT CAGGCC 3′ | NT 58810-58840 |
| | (SEQ ID NO:63) | |
| 8606 | 5′ CCCAAGCTTTGCACACCCTAGGGTATGTTCTTGCAATTC 3′ | NT 58500-58530 |
| | (SEQ ID NO:64) | |
| 8551 | 5′ CCCAAGCTTCCCAAAGCCATGCCCTGCTCCAGAAACACG 3′ | NT 56550-56580 |
| | (SEQ ID NO:65) | |
| 0003 | 5′ CCCAAGCTTATGATGTAACCCTCATTGGCCTCA 3′ | NT 54497-54520 |
| | (SEQ ID NO:66) | |
| 0006 | 5′ CCGGAATTCGAGACTCCAAGAAAAGATCTCATG 3′ | NT 55001-55024 |
| | (SEQ ID NO:67) | |
| 0007 | 5′ CCCAAGCTTATGTTCTTGCAATTCAGCGGAGGA 3′ | NT 58514-58537 |
| | (SEQ ID NO:68) | |
| 0008 | 5′ CCGGAATTCTGTGTGAGATCTCAGGAGAAGGTA 3′ | NT 58885-58908 |
| | (SEQ ID NO:69) | |

PCR Amplification of Rearranged VH, D and JH Segments from Genomic DNA

Genomic DNA was prepared using the Genoprep DNA isolation kit (Scientifix, Australia). Platinum PfX DNA polymerase (Invitrogen, Calif., USA) was used to amplify the rearranged VH, D and JH genes from genomic DNA. The PCR reaction included 1×PfX amplification buffer, 50 mM magnesium sulfate, 1×PCR enhancer solution, forward primer (10 pM), reverse primer (10 pM), dNTPs (10 mM each), template (100 ng), platinum PfX DNA polymerase (0.6 U) and sterile water in a final volume of 20 μl. Cycling conditions were as follows; one cycle of 95° C. for 5 minutes, 30 cycles of 95° C. for 30 seconds, 65° C. for 30 seconds, 68° C. for 2.5 minutes, and one cycle of 72° C. for 7 minutes. Primers used were specific for each of the seven VH family leader sequences together with a previously described consensus JH primer, JOL48, that anneals to all six human JH segments (Table 4). A second PCR reaction was performed using 0.5/20 μl of the first PCR as template to gain sufficient DNA for detection using ethidium bromide.

TABLE 4

Primers for PCR amplification of rearranged VH, D and JH segments
from genomic DNA.

Primer

| | | |
|---|---|---|
| Name | Sequence | Specificity |

| | | |
|---|---|---|
| 8111 | 5′ CCCAAGCTTATGGACTGGACCTGGAGGATCCTCTTCTTGGTGGCAGCA 3′ | VH1 |
| | (SEQ ID NO:113) | leader |
| 8116 | 5′ CCCAAGCTTATGGACACACTTTGCTCCACGCTCCTGCTGCTGACC ATCCCT 3′ | VH2 |
| | (SEQ ID NO:114) | leader |
| 8112 | 5′ CCCAAGCTTATGGAGTTTGGGCTGAGCTGGGTTTTCCTTGTTGCTATT 3′ | VH3 |
| | (SEQ ID NO:115) | leader |
| 8113 | 5′ CCCAAGCTTATGAAACACCTGTGGTTCTTCCTCCTGCTGGTGGCAGCT 3′ | VH4 |
| | (SEQ ID NO:116) | leader |
| 8118 | 5′ CCCAAGCTTATGGGGTCAACCGCCATCCTCGCCCTCCTCCTGGCTGTTCTC 3′ | VH5 |
| | (SEQ ID NO:117) | leader |
| 8114 | 5′ CCCAAGCTTATGTCTGTCTCCTTCCTCATCTTCCTGCCCGTGCTGGGCCTC 3′ | VH6 |
| | (SEQ ID NO:118) | leader |
| 8115 | 5′ CCCAACTTATGGACTGGACCTGGAGGATCCTCTTCTTGGTGGCAGCAGCA 3′ | H7 leader |
| | (SEQ ID NO:119) | |
| N8336 | 5′ CCGGAATTCGCGGTACCTGAGGAGACGGTGACC 3′ | Consensus |
| (JOL48) | (SEQ ID NO:120) | JH |

PCR products were run on 1.5% agarose gels and DNA was extracted from bands using Nucleospin Extraction Kit (Nagel-Macherey, Germany) according to manufacturer's instructions. A third PCR was performed on the purified DNA as described above. Products were cloned into pBluescript SK+ (Stratagene, Tex., USA) using EcoR I and Hind III sites and sequenced as previously described.

Identification of Rearranged VDJ Segment in RAMOS RA-1

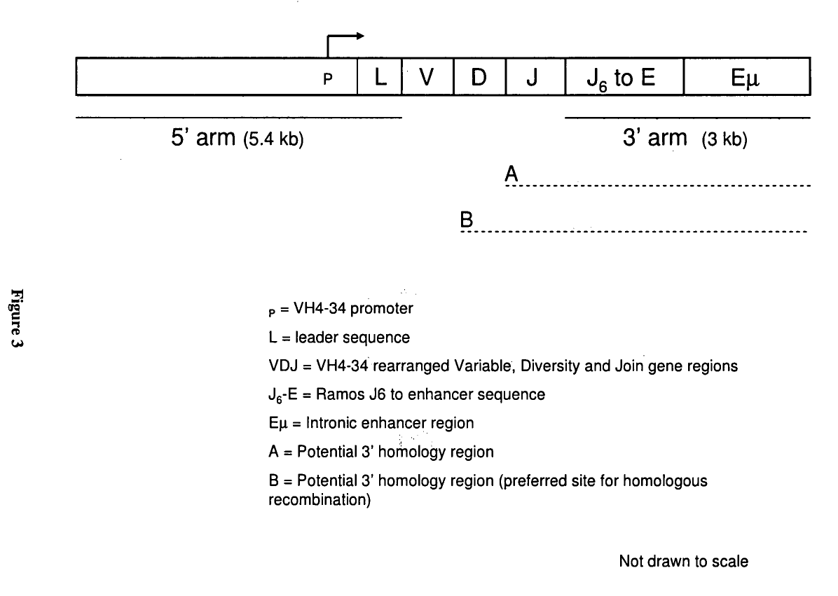

The sequenced VH allele for RAMOS RA-1 was identified as VH₄₋₃₄ (DP 63) using V-Base (http://www.mrc-cpe.cam.ac.uk/vbase). A schematic diagram of the gene targeting region is shown in FIG. 3 and the genomic sequence is set out in SEQ ID NO:1. The immunoglobulin. heavy chain promoter shown in FIG. 3 corresponds to nucleotides 4852-5190 of SEQ ID NO:1. The leader sequence (including intron 1) shown in FIG. 3 corresponds to nucleotides 5191-5329 of SEQ ID NO:1. The VDJ segment shown in FIG. 3 corresponds to nucleotides 5330-5708 of SEQ ID NO:1.

The consensus nucleotide sequence differs from the published VH₄₋₃₄ sequence (V-Base) by six nucleotides only (C₆₈→G, C₇₂→T, C₂₂₈→G, T₂₃₂→C, C₂₄₄→T, C₂₄₈→A) of which four were coding changes (A₂₃→G; N₇₆→K, F₇₈→L, T₈₃→N). The sequence was further classified as VH₄₋₃₄ subgroup 2 (Journal of Molecular Biology, 1987, Vol 195, 761-768) using the Kabat database (Kabat et al., 1991, Sequences of proteins of immunological interest, vol 1, 5^{th} edition). The C₆₈→G mutation in framework I and N₇₆→K mutation in framework 4 were unique to RAMOS RA-1 and occurred in otherwise conserved residues in the other four members of the group (Lee et al. 1987, Journal of Immunology, 142, 4054-4061, Sanz et al. 1988, Clinical Experimental Immunology 71, 508-516).

The nucleotide sequence generated from RAMOS DNA corresponding to the D allele differed significantly from the published alleles in V Base. Although no significant homology was identified, the closest related sequence similarity (and sequence length) was to D₃₋₁₆ (Corbett, S et al, Journal of Molecular Biology, 270, 587-597).

The sequenced JH allele for RAMOS RA-1 was identified as JH6b (Mattila et al. 1995) using V-Base. No nucleotide base changes were detected between the consensus sequence and the published JH_{6b} sequence.

Sequencing of the 3′ Region Downstream of the Rearranged VH Allele

The homologous sequence downstream of the site of integration chosen for the vector corresponds to a ˜3 kb region between the rearranged VJD genes through to the mu enhancer of the human immunoglobulin heavy chain locus of the RAMOS cell line (corresponding to sequence gi 29502084 nucleotides 960091-962947).

The region 3′ downstream of the rearranged VDJ segment was sequenced using methods previously described. Primers were designed based on published sequences, human-immunoglobulin heavy chain enhancer on chromosome 14 (gi 34819), human J6 to enhancer DNA of the immunoglobulin heavy-chain gene (gi 33100), human (AW-Ramos) translocated t(8;14) c-myc oncogene, exon 1 (gi 188910) and human mu switch DNA of the immunoglobulin heavy-chain gene locus (gi 33101)(Table 5).

Sequences were analysed using BLAST program (NBCI, http://www.ncbi.nlm.nih.gov/BLAST/) and assembled in Clone Manager Suite 7 (Scientific and Educational Software). The assembled sequence is set out as nucleotides 5709 to 8634 of SEQ ID NO: 1. This sequence shares 98% similarity with the published sequence gi 29502084 nucleotides 960091-962947.

Cloning of the 3 kb Fragment Downstream of the Rearranged VH Allele from Genomic DNA

The 3 kb fragment was amplified from genomic DNA extracted from RAMOS RA-1 cells using Platinum PfX DNA polymerase (Invitrogen, Calif., USA). The PCR reaction was the same as previously described except the forward primer 9779 (5′ CCGCTCGAGTGGGAGCCTCTGTGGATTTCCGA 3′) (SEQ ID NO:111) and reverse primer 9801 (5′ TGACCGGACGTCGCCCAGCCCAGCCTAGCTCA 3′) (SEQ ID NO:112) were used and the cycling conditions were as follows; one cycle of 94° C. for 15 seconds, fifteen cycles of 94° C. for 10 seconds, 68° C. for 2 minutes, 15 cycles of 94° C. for 10 seconds, 68° C. for 2 minutes with and an extra 15 seconds added each cycle, and one cycle of 72° C. for 7 minutes. A second PCR reaction was performed using 0.5/20.0 μl of the first PCR reaction as a template to gain sufficient DNA for detection using ethidium bromide.

TABLE 5

Primer sequences used for genomic PCR to sequence the 3 kb
region downstream of the rearranged VH allele (VH₄₋₃₄) in
RAMOS RA-1

Homologous

| | |
|---|---|
| Primer | sequence in NCBI |

| | | |
|---|---|---|
| Name | Sequence | database |

| | | |
|---|---|---|
| 8604 | 5′ CCCAAGCTTCGGCCCCGATGCGGGACTGCGTTTTGACCA 3′ | Gi 34819 NT 1-30 |
| | (SEQ ID NO:70) | |
| 8605 | 5′ CCGGAATTCATAACAAGCTAATTTAAAAAACTTTTTGAA 3′ | Gi 34819 NT 450-500 |
| | (SEQ ID NO:71) | |
| 8559 | 5′ CCCAAGCTTGCACAGACGGGAGGTACGGTATGGACGTCT 3′ | Gi 33100 NT 460-490 |
| | (SEQ ID NO:72) | |
| 8562 | 5′ CCGGAATTCAAAAAAATAAACTTGATTTATGAT GGTCAA 3′ | Gi 33100 NT 705-734 |
| | (SEQ ID NO:73) | |
| 8603 | 5′ CCGGAATTCCGCGGTGACCTGCTTCCTGCCACCTGCTGT 3′ | Gi 34819 NT 126-155 |
| | (SEQ ID NO:74) | |
| 8692 | 5′ CCGGAATTC AGTTAGTGCAGCCAAGCCCT 3′ | Gi 33101 NT 301-320 |
| | (SEQ ID NO:75) | |
| 8695 | 5′ CCGGAATTCAAAAGGCAAGTGGACTTCGGTGCTTACCTG 3′ | Gi 188910 NT 945-974 |
| | (SEQ ID NO:76) | |
| 8697 | 5′ CCCAAGCTT CAGCTCAGCTCAGTTCAGTTCAGCCCT 3′ | Gi 188910 NT 4-30 |
| | (SEQ ID NO:77) | |
| 8508 | 5′ CCCAAGCTTATGCGAGGGTCTGGACGGCTGAGGACCCCC 3′ | Gi 188910 NT 370-400 |
| | (SEQ ID NO:78) | |
| 8696 | 5′ CCGGAATTCATGCGGCAAGGGTTGCGGACCGCTGGCTGG 3′ | Gi 188910 NT 715-744 |
| | (SEQ ID NO:79) | |
| 8694 | 5′ CCGGAATTCGCCCAGCCCAGCCTAGCTCA 3′ | Gi 33101 NT 770-790 |
| | (SEQ ID NO:80) | |
| 8691 | 5′ CCCAAGCTTTATCAACTGCTAGTTTGTG 3′ | Gi 34819 NT 361-381 |
| | (SEQ ID NO:81) | |
| 9090 | 5′ CCGGAATTCAGGGCTGAACTGAACTGAGCTGAGCTG 3′ | Gi 188910 NT 1-30 |
| | (SEQ ID NO:82) | |
| 9879 | 5′ CGGCTGATATCTGGGAGCCTCTGTGGATTTTCCGA 3′ | Gi 33100 NT 1-24 |
| | (SEQ ID NO:83) | |
| 9805 | 5′ AGCCGGATATCGCCCAGCCCAGCCTAGCTCA 3′ | Gi 33101 NT 770-790 |
| | (SEQ ID NO:84) | |
| 0002 | 5′ GAAAGTTAAATGGGAGTGACCCAG 3′ | GI 29502084 NT |
| | (SEQ ID NO:85) | 962860-962884 |
| 0021 | 5′ GAGTGACCATCGCACCCTTGACAG 3′ | Gi 29502084 |
| | (SEQ ID NO:86) | |

The 3 kb fragment was cloned into pPCRScript using the PCR-Script Amp cloning kit (Stratagene, Tex., USA) at the Srf I site. The resulting construct was referred to as 3 kbPCRScript 10-1-3 (5822 bp). This construct was sequenced using primers in Table 5.

The size and GC rich stretches present in the 5 kb and 3 kb homology regions gave rise to structural regions which caused difficulties in cloning and thereafter problems with stability of the-cloned regions.

### Example 2

### Design and Construction of a Vector for Integration into the Rearranged VH Allele, VH4-34 in RAMOS RA-1

1) Construction of Vector for Integration

A 3 kb fragment, containing sequence homologous to the region downstream of the rearranged allele VH₄₋₃₄ was amplified from the construct 3 kbPCRScript 10-1-3 with the forward primer 9879 (5′ CGGCTGATATCTGGGAGCCTCTGTGGATTTTCCGA 3′) (SEQ ID NO:83) and the reverse primer 9805 (5′ AGCCGGATATCGCCCAGCCCAGCCTAGCTCA 3′) (SEQ ID NO:84) using Platinum PfX DNA polymerase (Invitrogen, Calif. USA). PCR products were purified using QIAquick PCR Purification Kit (Qiagen™) according to manufacturer's instructions then digested with EcoRV and ethanol precipitated. Digested fragments were ligated into construct 5 kbPCRScript 15a-7 containing the 5 kb sequence upstream of the rearranged VH allele. The DNA was transformed into Escherichia coli and grown at 37° C. overnight.

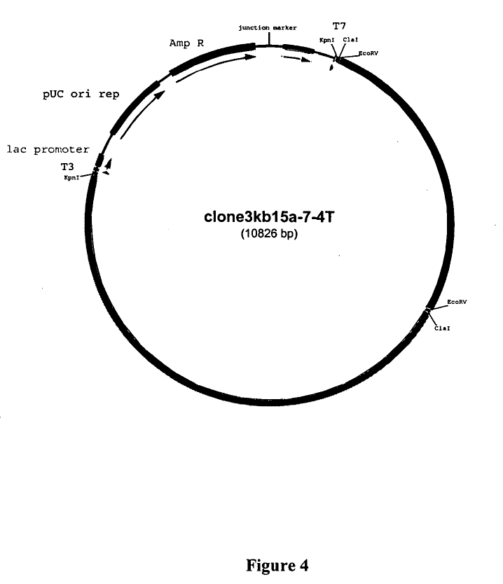

Bacterial colonies were screened by Southern blotting and probed using ³²P labeled oligonucleotides 8604 (5′ CCCAAGCTTCGGCCCCGATGCGGGACTGCGTTTTGACCA 3′) (SEQ ID NO:70) to detect the 3 kb fragment and a pool of labeled oligonucleotides 8687 (5′ CCCAAGCTTCATGTTCCACGCATTACGTC 3′) (SEQ ID NO:54), 8440 (5′ CCGGAATTCAATTFGAGATTGTGTGTGAGATCTCAGGAG 3′) (SEQ ID NO:38) and 8472 (5′ CCGGAATFCGTFGGGTFCCCAGTGTAGGTGATGATCCAT 3′) (SEQ ID NO:44) to detect the 5 kb fragment. Colonies that were positive for both fragments were subcultured and DNA was extracted using QIAprep Miniprep kit (Qiagen™). Clones were analysed by diagnostic restriction enzymes and PCR for each fragment and sequenced using an ABI 373 DNA sequencer, with T7 and T3 primers. This new construct is referred to as 3 kb15a-7-4T is 10 826 bp long (FIG. 4). The sequence of is shown in SEQ ID NO:87.

2) Transfection of3 kb15a-7-4T into RAMOS and Screening for Integration

Clone 3 kb15a-7-4T (5 μg) is transfected into 3×10⁶ RAMOS RA-1 cells using the following protocol; cells are centrifuged to remove spent media then resuspended at 1×10⁶ cells/ml in RPMI containing DEAE Dextran (25 μg/ml). Resuspended cells (1 ml) are transferred into electroporation cuvettes (4 mm gap), and DNA is added. The cuvette containing the cell/DNA mixture is then incubated at 37° C. for 10 minutes. Cells are then pulsed twice at 550 V and 25 μFd capacitance, using a Gene Pulser (BioRad). Following a 10 minute incubation on ice, the cells are removed from the cuvette and transferred to T₂₅ flasks containing 9 ml of RPMI+15% FCS. Flasks are incubated at 37° C. for at least 24 hours before the efficiency of transfection is assessed. Mock transfected cells are used as controls.

Three days after transfection, cells are stained for surface IgM and sorted by flow cytometry into an IgM positive population and IgM negative population. Prior to this experiment the background level of IgM negative cells is determined.

Genomic DNA is extracted from IgM negative cells using the Genoprep DNA isolation kit (Scientifx, Australia) according the manufacturers' instructions. The DNA is then digested using Xba I enzyme and run on a 0.6% agarose gel. DNA is transferred onto nitrocellulose membrane using standard methods for Southern blotting and probed with ³²P labeled oligonucleotides 0041 (5′ GACGGTATCGATAAGCTTGATATCGAATFCCTGCAGCCCGGG 3′) (SEQ ID NO:88) and 0042 (5′ GACCTCCTGCACAAGAACGGTACCGGGCTAGAGCGGCCGCCA 3′) (SEQ ID NO:89) that span the junction regions. A probe against the middle of the sequence that would be integrated, 9403 (5′ CAGTGCTGCAATGATACCGCGAGAC 3′) (SEQ ID NO:90) is also used.

The following patterns of radioactive probe binding to DNA extracted from cells transfected with i) vector only, no radioactive signals ii) the construct containing the 3 kb15a-7-4T shows radioactive signals with all three radioactive probes as is expected when the 5 kb and 3 kb recombination occurs with the chromosomal DNA integrating the middle sequence; the binding of the radioactive 9403 probe shows this iii) DNA from untransfected cells show no binding with the 9403 radioactive probe, iv) the lanes on the agarose gel with the plasmid 3 kb15a-7-4T show radioactive signal with all probes. Obtaining this pattern i-iv) is indicative of 3 kb15a-7-4T integration into the host cell genome.

### Example 3

### Design and Construction of a Vector for Integration into the Rearranged VH Allele, VH₄₋₃₄ in RAMOS RA-1 and Mutation of the asFP499 Gene

The components of the vector for integration are as follows:

i) Construct 5 kb in PCRScript 15a-7 is modified by removing the Nae I-Xho I fragment which effectively deletes an additional Kpn I site. This new construct is herein referred to as 5 kbPCRScript minus Nae I-Xho I and is 7608 bp in length.

ii) The cloned gene for asFP499, which is a fluorescent protein isolated from the sea anemone Anemonia sulcata, was obtained from J. Wiedenmann (University of Ulm, Germany) in the plasmid pQE32 (Qiagen, Calif. USA). Four sequential PCR reactions were performed to introduce restriction sites Kpn I and Not I at the, 5′ and 3′ ends of the asFP499 gene respectively and two C-terminal flag tags with a Sal I site at the 3′ end of the second flag tag. This final product (˜770 bp) is subcloned into pPCRscript (Stratagene, Tex., USA) and herein is referred to as target PCRScript as FP499-1.

iii) The gene encoding thymidine kinase is amplified by PCR and restriction sites Hind III and Cla I are introduced at the 5′ and 3′ end of the gene respectively. This product (1260 bp) is subcloned into pPCRscript (Stratagene, Tex., USA) and herein is referred to as construct TKPCRscript -64.

iv) pMC1neo Poly A (3800 bp) was obtained from Stratagene (Tex., USA).

These components are assembled in the following order:

Constructs targetPCRScriptasFP499-1 and 5 kbPCRScript minus Nae I-Xho I are digested with Kpn I and Sal I and run on 1.0 and 0.9% agarose gels respectively. The desired products (˜7608 bp and ˜770 bp) are cut out and DNA extracted using QIAquick gel extraction kit (QiagenTM). The asFP499 gene (˜770 bp) is ligated into 5 kbPCRScript minus Nae I-Xho I (˜7608 bp) to create 5 kb-asFP499PCRscript minus Nae I XhoI (8289 bp).

Constructs TKPCRscript-64 and 5 kb-asFP499PCRscript minus Nae I-XhoI are subsequently digested with Cla I and Sal I and run on agarose gels and DNA is extracted as described above. The asFP499-5 kb fragment (˜5770 bp) is ligated to the TKUCRScript-64 backbone (˜4737 bp) to generate TK-5 kb-asFP499 PCRscript (10464 bp).

The TK-5 kb-asFP499 cassette (˜7558 bp) is. digested out of TK-5 kb-asFP499 PCRscript with Sal I and Hind III and ligated into pMC1neo Poly A, cleaved with Sal I and Hind III (3837 bp). This new construct is designated KW 1.

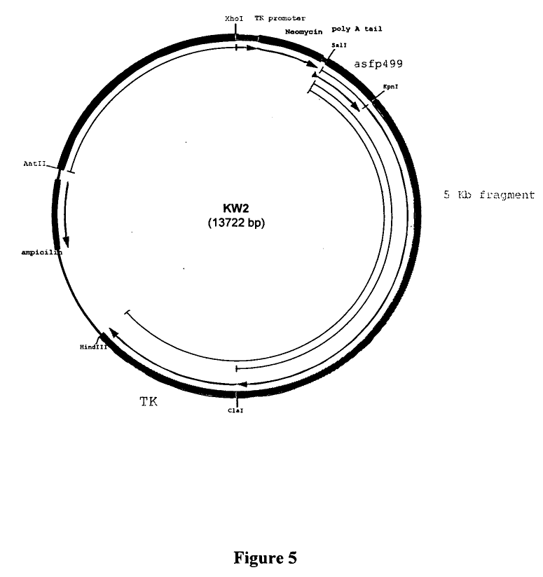

Finally, 3 kbPCRscript 10-1-3 and KW1 are digested with restriction enzymes Xho I and Aat II and gel purified as described above. The 3 kb fragment is ligated into the KW1 backbone (˜10868 bp) yielding'the final integration-vector designated KW2 (13722 bp) (FIG. 5). The sequence of integration vector KW2 is set out in SEQ ID NO:91.

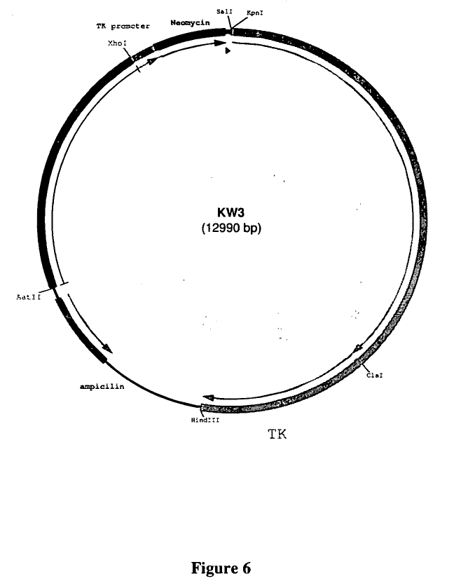

The asFP499 gene was deleted from vector KW2 to generate vector KW3 (FIG. 6). The sequence of integration vector KW3 is set out in SEQ ID NO:110. It will be appreciated that any target nucleic acid molecule of interest can be inserted into this vector for use in the affinity maturation process of the present invention.

### Example 4

### Optimal Culturing Conditions for RAMOS RA-1 Cells

Optimal growth conditions for RAMOS cells were determined by performing growth curve experiments using different supplements to the medium and different seeding concentrations. RAMOS cells were seeded at 1×10⁴, 5×10⁴, 1×10⁵, and 2×10⁵ (cells/ml) and cultured as 25 ml cultures in either RPMI medium with 10% heat inactivated FBS and 1 mM sodium pyruvate or RPMI medium with 15% heat inactivated FBS, I mM sodium pyruvate and 50% conditioned medium. Every 24 hours, 1 ml samples of cells were taken and the number of viable cells was determined using the Vi-Cell Viability. Analyser (Beckman Coulter, Calif., USA,). The results showed that RAMOS cells had a lag phase of 48 hours regardless of the seeding concentrations. Cultures reach an exponential growth phase at a density of 0.25-0.5×10⁶ cells/ml RPMI medium with 10% heat inactivated FBS and 1 mM sodium pyruvate whereas cells growing in RPMI medium with 15% heat inactivated FBS, 1 mM sodium pyruvate and 50% conditioned medium did not enter an exponential phase. The optimal seeding rate for RAMOS was 1×10⁵ cells/ml.

Mycoplasma infection can affect transfection rates, therefore we tested RAMOS RA-1 monthly. Cells were tested using 4′, 6-diamidino-2-phenylindole (DAPI) staining in which all DNA is stained specifically with this very bright dye. If mycoplasma is present small bright specks of dye are seen in the cytoplasm. Cells were fixed onto glass slides and viewed under a 100× objective with a UV filter system. Cells were negative for mycoplasma over a period of 12 months.

### Example 5

### RAMOS RA-1 Cell Division Rate

Transfection rates in RAMOS are affected by the viability of cells in culture. To identify, the optimal time during the growth cycle to transfect RAMOS cells, we first determined the cell division rate. RAMOS cells were stained with cell-permeant fluorescein-based dye carboxyfluorescein diacetate succinimidyl ester (CFSE) and analysed by flow cytometry based on established techniques in Current Protocols in Cytometry 9.11.2.

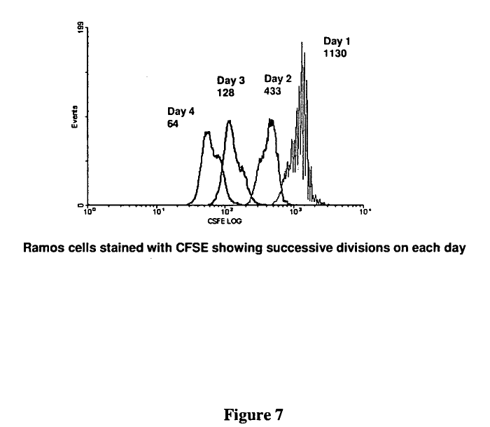

Briefly, RAMOS cells in exponential log phase growth were washed and re-suspended in phosphate buffered saline at 5×10⁶ cells/ml. A volume of 2 μl of 5 mM CFSE was added per ml of cells. Cells were incubated for 10 minutes at 37° C. after which 5 volumes of ice cold RPMI+10% FBS was added. Cells were then incubated on ice for a further 5 minutes and washed three times in culture medium before transferring to flasks and culturing under normal conditions (see example 1). Initially cells were not analysed until 12 hours post staining to allow. for the initial fluorescence decay. Thereafter cells were analysed by flow cytometry every 24 hours. The CFSE fluorescence was plotted against the number of cells analysed (FIG. 7). Upon cell division, the dye is equally distributed between daughter cells, allowing the resolution of up to eight cycles of cell division by flow cytometry. In the case of using cultured cells that divide in concert, the resulting fluorescent distribution is halved per cell after each division. Therefore, these results indicate that the cell population was dividing at least once every 24 hours.

### Example 6

### Optimisation of Conditions for Transfection of RAMOS RA-1 Cells

1) Construction of Vector for Expression of asFP499 in RAMOS RA-1

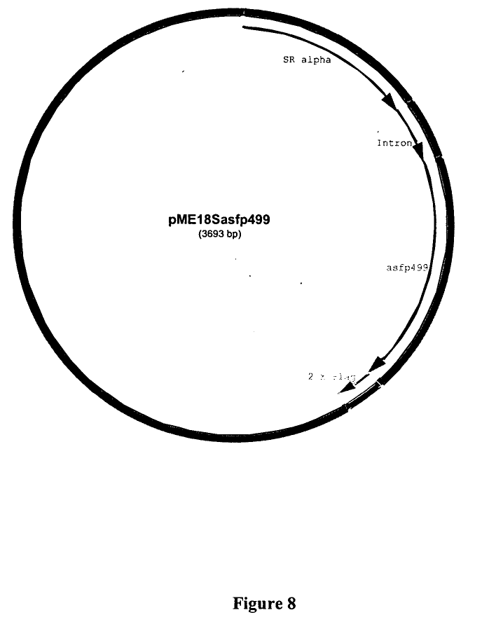

Primers were designed to add a Xho I site to the 5′ end of the asFP499 gene and two flag tags, two stop codons and a Xba I to the 3′ end of the gene to allow cloning into pME18s. The primers used are 8934 (5′ CCGCTCGAGATGTATCCTrCCATCAAGGAAACC 3′).(SEQ ID NO:92), 8935 (5′ TCTAGATTATTATTTATCATCATCATCTTTATAATCTTTATCATCATCATCTTTATAATCAGCGGCCGC 3′ (SEQ ID NO:93); 8936 (5′ CTAGTCTAGATTATTATTTATCAT,CATCATC 3′) (SEQ ID NO:94), and 8398 (5′ GTTATGTCCTAATTTCGAAGGCACTTGGGAGTA 3′) (SEQ ID NO:95). The cloned sequence was confirmed by DNA sequence analysis. The resulting construct, referred to as pME18sasFP499 (3693 bp) (FIG. 8) (SEQ ID NO:8) was used for subsequent transfection of RAMOS cells.

ii) Transfection of pME18sasFP499 into RAMOS RA-1 Cells

A number of different transfection reagents or methods can be used to transfect mammalian cells including Calcium Phosphate coprecipitation, Lipofectamine (Invitrogen), FuGENE6 (Roche, Germany), SuperFect (Qiagen, Calif., USA), Effectene (Qiagen, Calif., USA), GenePORTER (Gene Therapy Systems, Calif., USA) and Metafectene (Biontex, Germany). Electroporation is another method commonly used to transfect mammalian cells. Electroporation can be carried out using different electroporators such as the Gene Pulser (BioRad, Calif., USA) or the Electro Square Porator ECM 830 (BTX, Fisher Biotech, Australia) and various voltage and capacitance settings can also be used. Since RAMOS RA-1 cells are of B-lymphoid origin and it is known that lymphoid cells can be difficult to transfect, we optimised transfection of this cell line.

The efficiency of transfection of RAMOS RA-1 cells was monitored by flow cytometric analysis using an EPICS Elite (Beckman Coulter, Calif., USA). Samples of transfected cells were stained with 1 μg/mL Propidium Iodode (PI). The live cell population (based on forward and side scatter characteristics and PI staining) was gated and the percentage of Fluorescent Protein (FP) positive cells was assessed. FP expression was also assessed by fluorescence microscopy using an Olympus IX70 microscope and Olympus U-RFL-T burner.

In order to optimise the transfection process, a number of different transfection parameters were tested including: set voltage, set capacitance, amount of DNA, concentration of DEAE Dextran and analysis time post transfection.

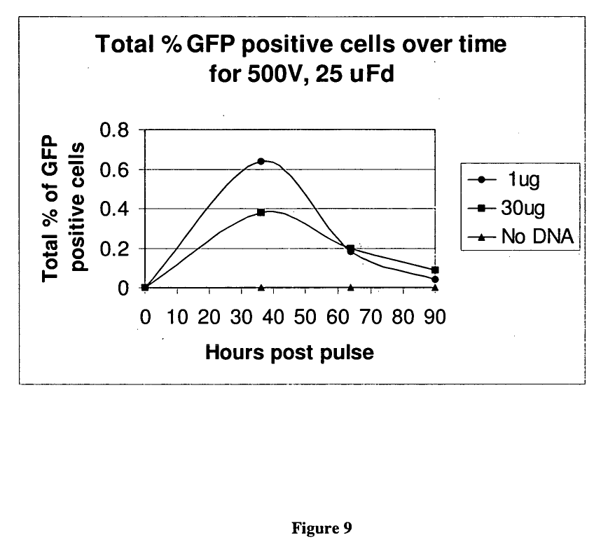

FIGS. 9 show the results of a representative set of optimisation experiments. The combination of 550 V and 25 μFd resulted in the highest transfection efficiency. No significant effect on the transfection efficiency was observed using 1 μg or 30 μg of pME18sEGFP. The optimum time to analyse FP expression was between 24 and 36 hours post electroporation. DEAE Dextran varying from 25 μg/ml to 1 mg/ml was tested and concentrations between 25 μg/ml and 100 μg/ml were optimal for transfection but concentrations above 500 μg/ml were found to be toxic to the cells (data not shown).

Using the optimum conditions, transfection rates of between 0.5% and 3.5% were achieved with the average being approximately 1.0%.

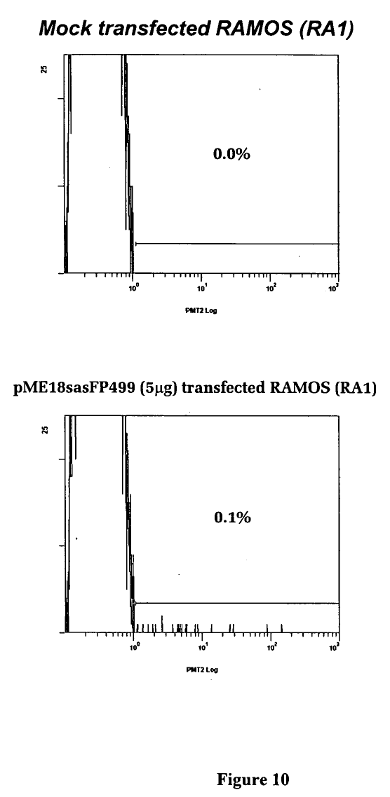

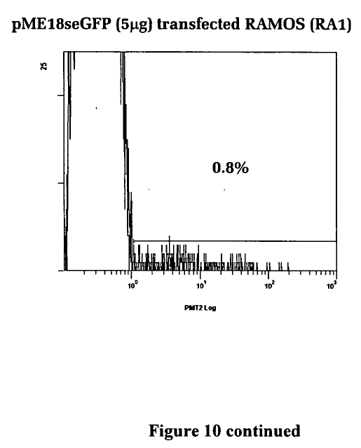

FIG. 10 shows the comparison of RAMOS RA-1 cells transfected with pME18sEGFP, pME18sasFP499 or mock transfected. The level of expression of asFP499 in RAMOS RA-1 was assessed 24 hours post transfection and was found to be approximately 10 fold lower than that of EGFP.

### Example 7

### Quantitation of the Number of IgM Molecules on the Surface of RAMOS RA-1 Cells

It is known in the art that the loss of surface IgM may be used to monitor integration into the rearranged VH allele.

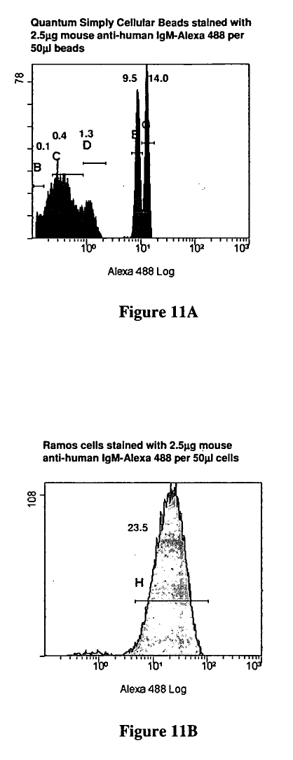

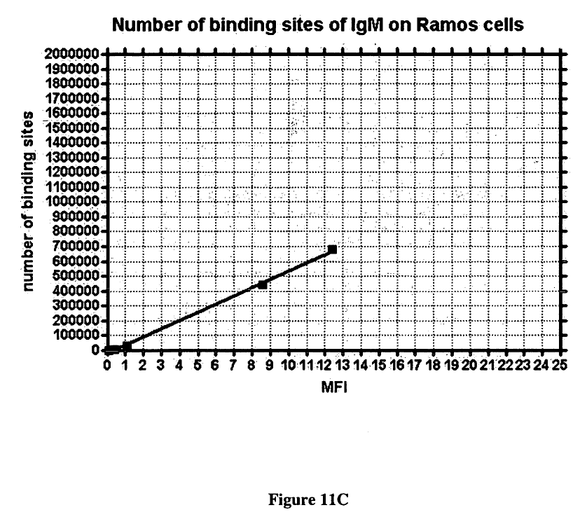

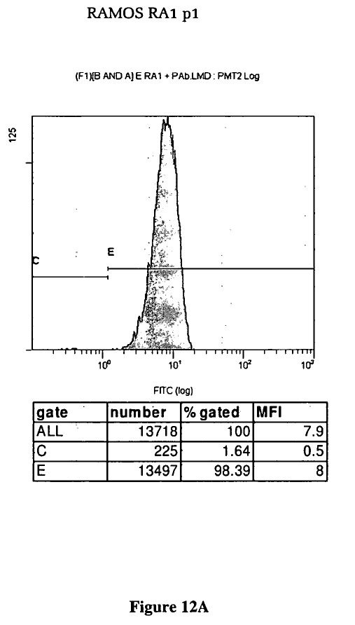

We therefore determined the number of natural IgM molecules on the surface of RAMOS cells. RAMOS cells (50 μl) and Quantum Simply Cellular™ beads (Bangs Laboratories, IN, USA) (50 μl) were separately incubated on ice for 1 hour with a saturating amount (2.5 μg) of a mouse anti-human IgM monoclonal antibody (Southern Biotech, AL, USA) conjugated to Alexa Fluor 488™ (Molecular Probes, OR, USA). Cells and beads were then analysed by flow cytometry using an EPICS Elite (Beckman-Coutler). Fluorescence intensity (at 488 nm) was plotted against the number of cells or beads analysed (FIGS. 11a and 11b). Five distinct populations of beads (labeled B, C, D, E, G) which have defined numbers of anti-mouse IgG binding sites (0, 2000, 20 000, 46 000 and 68 000) were observed (FIG. 11a). FIG. 11b (H) shows a normal distribution of fluorescence intensity, ranging from 2 to 100, as expected for a population of cells. The mean fluorescence for each of these populations was plotted against the corresponding number, of predetermined binding sites to generate a linear line graph (FIG. 11c) from which we extrapolated the average number of IgM molecules on RAMOS cells. The mean fluorescence intensity was calculated as 23.5 which corresponded to 1.25×10⁶ molecules for RAMOS RA-1.

### Example 8

### Monitoring Cell Surface IgM Loss on RAMOS RA-1

RAMOS RA-1 cell surface IgM decreases with time in culture. We therefore established the rate of natural loss of surface IgM in order to use this characteristic as a marker for integration. To evaluate the percentage of the population that were IgM positive, RAMOS RA-1 cells were stained for IgM and analysed by flow cytometry.

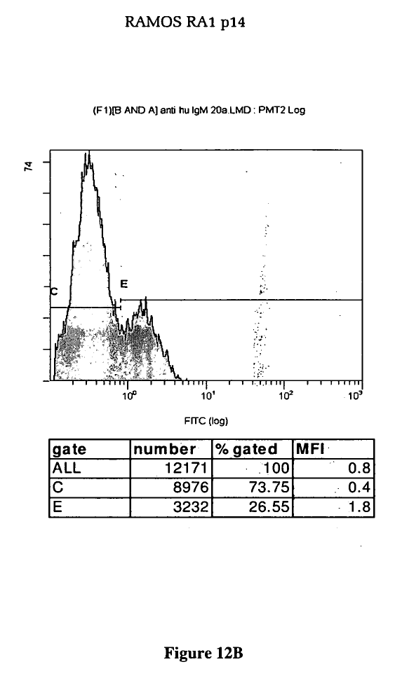

Briefly, cells were washed and resuspended at 1×10⁶ cells/100 μl. A sheep polyclonal antibody against human IgM (μ chain specific) FITC conjugated (Chemicon, Calif., USA) (10 μl) was added to cells which were incubated on ice for 1 hour. Cells were then washed with 2 ml of cold wash buffer (PBS+2% FBS, 0.01% sodium azide) and resuspended in 500 μl this buffer with 5 μl propidium iodide (1 mg/ml) (Sigma-Aldrich, Australia). Cells were analysed by flow cytometry using an EPICS Elite (Beckman Coulter, Calif., USA). Cells were gated on the basis of forward and side scatter and negative propidium iodide. In passage one of RAMOS RA-1 98.39% of the cell population was positive for surface IgM (FIG. 12a) by passage 14, 26.55% of the cell population was positive (FIG. 12b). This loss is attributed to either a high mutation rate or a growth advantage of IgM negative mutants. Therefore, RAMOS RA-1 cells were presorted to remove IgM negative cells and the IgM⁺ cells quantitated prior to transfection as the IgM loss is used as a measure of integration.

To further investigate IgM loss on RAMOS, we quantitated the number of cell surface IgM molecules using the methodology described above. We observed that the average number of cell surface IgM molecules. significantly decreased over time. In passage 4, RAMOS RA 1 cells possessed an average 9.0×10⁵ molecules of IgM on their surface, however by passage 16 the number of IgM molecules had decreased to 4.1×10⁵ molecules (FIG. 12). Together these data indicate that the decrease in cell surface IgM is dependent on passage number.

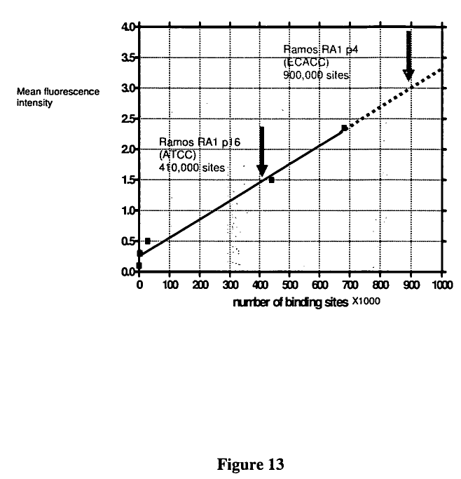

We have also observed and quantitated differences in the rate of IgM loss between RAMOS strains, ‘RAMOS RA-1’ supplied by American Tissue Culture Collection and ‘RAMOS’ supplied by European Collection of Cell Culture (ECAC) (FIGS. 13). Together these data suggest that although IgM loss is a marker for integration reported widely in the literature, we have opted to use an antibiotic marker, such as neomycin as a positive selection marker and thymidine kinase as a negative selection marker for integration in our system.

### Example 9

### Surface Display of asFP499 using the Anchor Domain of CD26

For cell surface display of the asFP499 gene product on RAMOS cells we used the transmembrane domain of CD26 (Tanaka, T et al., 1992, J. Immunol. 149 (2), 481-486) as an anchor.

Transmembrane domain of CD26 Accession No: M74777 (gi 180082)

| | |
|---|---|
| Peptide sequence: | |
| MKTPWKVLLGLLGAAALVTIITVPVVLLNK | (SEQ ID NO:96) |
| Nucleotide sequence: 10-100 | |
| 5′ ATGAAGACACCGTGGAAGGTTCTCCTGGGACTGCTGGGTGCTGCTGCGC | (SEQ ID NO:97) |
| TTGTCACCATCATCACCGTGCCCGTGGTTCTGCTGAACAAA 3′ | |

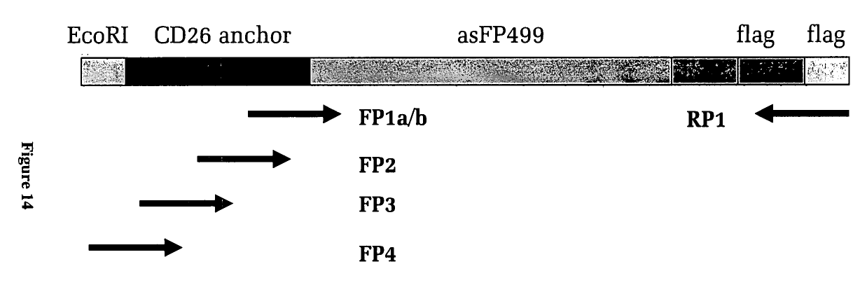

This anchor sequence was added to the 3′ end of the asFP499 gene, to provide an N-terminal anchor on the protein (CD26asFP499). This was achieved by sequential overlap extension PCR using primers that partially overlapped the existing DNA end and also added new sequence. A representation of this sequential overlap PCR is shown in FIG. 14. This method can be used to add sequence to either the 3′ or 5′ end of a sequence. Primers are listed in Table 6.

TABLE 6

Primers used for addition of CD26 anchor sequence to asFP499

| | |
|---|---|
| Primer | Sequence |

| | |
|---|---|
| 9712 | 5′ CGTGCCCGTGGTTCTGCTGAACAAAATGTATCCTTCCATCAAGGAAACCA 3′ |
| | (SEQ ID NO:98) |
| 9713 | 5′ CCGTGCCCGTGGTTCTGCTGAACAAAGTGTATCCTTCCATCAAGGAAACCA 3′ |
| | (SEQ ID NO:99) |
| 9726 | 5′ TGCTGCTGCGCTTGTCACCATCATCACCGTGCCCGTGGTTCTGCTGAACAAA 3′ |
| | (SEQ ID NO:100) |
| 9727 | 5′ GGAAGGTTCTCCTGGGACTGCTGGGTGCTGCTGCGCTTGTCACCATCATCA 3′ |
| | (SEQ ID NO:101) |
| 9728 | 5′ CCGGAATTCATGAAGACACCGTGGAAGGTTCTCCTGGGACTGCTGGG 3′ |
| | (SEQ ID NO:102) |
| 9963 | 5′ GACTAGTTTATTATTTATCATCATCATCTTTATAATCTTTATCATCATC 3′ |
| | (SEQ ID NO:103) |

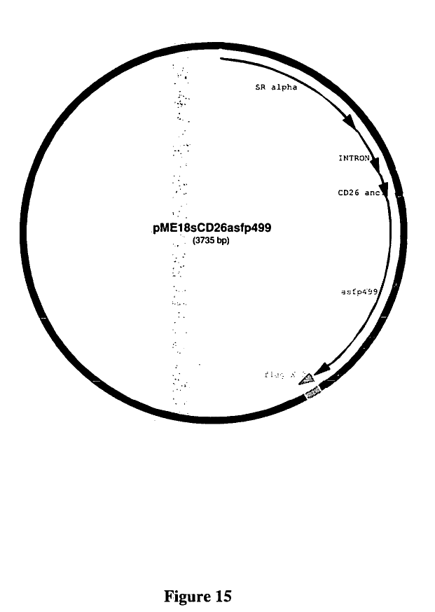

The final PCR product was cloned into pME18s as described previously except that the restriction sites SpeI and EcoRI were used. The cloned sequence was confirmed by DNA sequence analysis. The resulting vector was designated pME18sCD26asFP and is shown in FIG. 15. The sequence of pME18sCD26asFP is set out in SEQ ID NO:104.

### Example 10

### Transfection and Analysis of CD26asFP499

To demonstrate cell surface display of CD26asFP499, the plasmid pME18sasFP499CD26 was transfected into RAMOS RA-1 and control HEK 293T cells.

RAMOS RA-1 cells were transfected as previously described (Example 6). Transfections in HEK 293T cells, which were 60% -90% confluent, were carried out using FuGENE6 (Roche, Germany) according to the manufacturer's instructions. 5 μg of DNA was used in each transfection and pME18sasFP499 was used as a positive control.

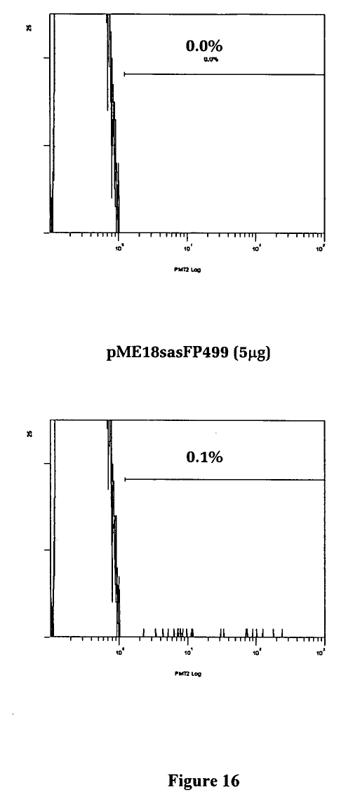

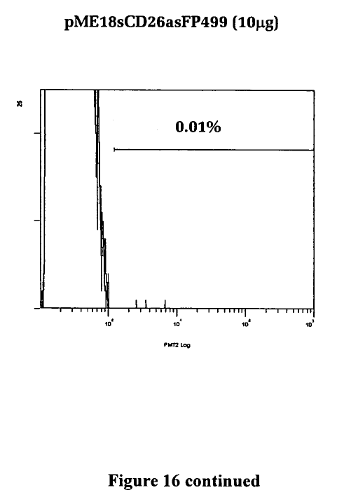

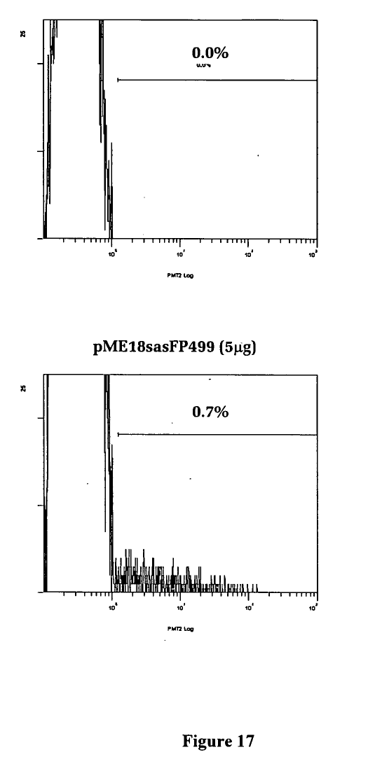

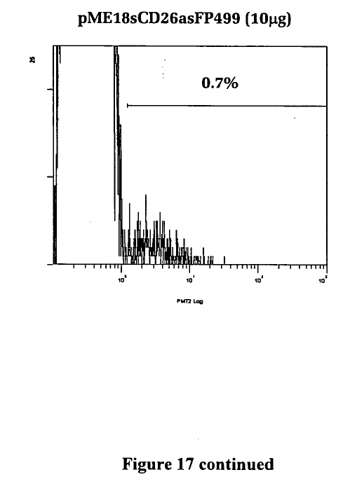

Efficiency of transfection was assessed by flow cytometry as previously described. FIG. 16 shows the flow cytometry data for transfection of RAMOS RA-1 cells with pME18sCD26asFP499. It can be seen that the efficiency of transfection with this vector in this cell line is very low (0.01% corresponds to 4 positive cells in this case). The data for transfection of HEK 293T cells is shown in FIG. 17. In this cell line, the transfection efficiency of pME18sCD26asFP499 is equal to that of pME18sasFP499 and the expression of both vectors is improved in this cell line. The mean fluorescence intensity of pME18sCD26asFP499 was lower than that of pME18sasFP499.

HEK 293 T cells transfected with pME18sCD26asFP499 were analysed by confocal microscopy using a Nikon C1 (Coherent Life Sciences, Australia). The majority of cells showed a diffuse fluorescence at their periphery indicating expression of asFP499CD26 at the cell membrane, whereas, bright fluorescence was observed uniformly throughout control cells transfected with pME18sasFP499 (data not shown).

### Example 11

### Cloning of the Coding Region of AICDA (AID) Gene into pME18s

Activation Induced Cytidine Deaminase (AID), the protein product of the differentiation specific AICDA gene has been shown to be a B-cell-specific factor required and essential for the processes of Class Switch Recombination (CSR) and Somatic Hypermutation (SHM) in B-cells. Its ectopic expression in a number of mammalian cell systems including non B-cell systems has shown the ability of AID protein to induce and/or enhance hypermutation (Martin et. al 2002, Okazaki et. al 2002, Yoshikawa et. al 2002).

Extraction of Total mRNA from RAMOS RA-1

Cells were harvested and centrifuged at 1500 rpm and resuspended in PBS. Total cellular mRNA was extracted using a GenoPrep™ mRNA isolation kit (Scientifix, Australia) according to the manufacturer's instructions. Briefly, after removing the supernatant, 700 μl of Lysis and Binding solution was added to cells (1×10⁶). This mixture was combined with 50 μl (250 μg) of GenoPrepTM mRNA magnetic beads and incubated at room temperature for 5 minutes. Beads were magnetically collected and washed with 500 μl of washing solution I. Beads were then washed twice with 500 μl of washing solution II. mRNA-bead complexes were resuspended in 20 μl sterile water and then incubated at 65° C. for 2 minutes. Beads were magnetically collected and the mRNA-containing supernatant was transferred to a new tube.

The human AICDA (AID) gene was amplified from RAMOS RA-1 total RNA using the Superscript One-step RT-PCR with platinum Taq kit (Invitrogen, Calif. USA). The reaction included 1× reaction mix, forward primer 9645 (5′ ATGGACAGCCTCTTGATGAACCGGAGGA 3′) (SEQ ID NO:105) 10 pM, reverse primer 9646 (5′ CAAAGTCCCAAAGTACGAAATGCGT 3′) (SEQ ID NO:106) 10 pM, template RNA (˜150 ng), RT/Taq mix (1.0 μl) and sterile water in a final volume of 50 μl. Cycling conditions were as follows; one cycle of 55° C. for 30 minutes, 94° C. for 2 minutes, 35 cycles of 94° C. for 30 seconds, 55° C. for 30 seconds, 68° C. for one minute, and one cycle of 72° C. for 7 minutes.

The RT-PCR product (596 bp) was amplified by PCR using Platinum Pfx polymerase (Invitrogen Calif., USA) as previously described This product was then cloned into pPCRScript using the PCR-Script Amp cloning kit (Stratagene, Tex., USA) at the Srf I site. The coding region of AID was then subcloned into the pME18s using Xho I and Xba I restriction sites with primers 9792 (5′ CCCTCGAGATGGACAGCCTCTTGATGAACCGGA 3′) (SEQ ID NO:108) and 9793 (5′ GCTCTAGACAAAGTCCCAAAGTACGAAATGCGT 3′) (SEQ ID NO:109). The PCR reaction and cycling conditions were as described above. These constructs were verified by sequencing as previously described. The DNA sequence of the coding region of the AICDA gene is set out in SEQ ID NO:107.

### Example 12

### Affinity Maturation of an Antibody Fragment

The gene targeting vector KW2 is modified such that following integration and target nucleic acid expression, a chimeric protein consisting of an antibody fragment fused to an anchor is produced. This is achieved by using standard molecular biology techniques to clone the CD26 anchor sequence into KW2 and then inserting the sequence for the antibody fragment downstream of the CD26 anchor sequence. The resulting vector is called KW3.

RAMOS RA-1 cells are transfected with the gene targeting vector KW3, using the optimised protocol previously described. The cells are allowed to recover for 48 to 72 hours before G418 at 5 mg/ml and gancyclovir or FIAU are added to the media for 7 to 9 days. This results in the selection of stable transfectants and also allows time for mutation and surface display to occur. The live cell population, which consists of the stable transfectants, is sorted using flow cytometry and allowed to react with the fluorescently labelled binding partner of the displayed antibody fragment. The cells with the highest fluorescence intensity (ie highest binding) are then single cell sorted into 96 well U bottomed plates containing 100 μl of 50% conditioned media and 50% fresh growth media. Single cell sorting is achieved using the Autoclone function of the EPICS Elite (Beckman Coulter, Calif., USA).

It is also possible to select the cells with the highest affinity gene products on the cell surface, by capturing cells with immoblised binding partner, and selecting for the highest affinity gene product by competitive elution.

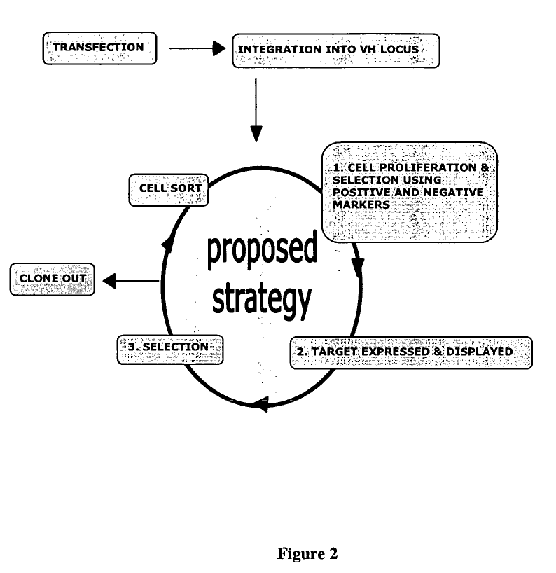

If necessary, the cells can be cycled through the in vivo strategy (FIG. 2). The single cells can be expanded to between 1×10⁵ and 1×10⁶ to allow further mutation to occur and then reacted with the labelled binding partner and single cell sorted again. This cycle of single cell sorting/expansion and mutation/re-selection can be carried out until cells displaying the molecule with the desired characteristic, in this case increased binding affinity, have been isolated. The DNA from these cells can be extracted and the mutated gene can be amplified by PCR. Alternatively, the RNA can be extracted and the gene amplified by RT-PCR. The amplified gene can then be inserted into an appropriate expression vector for high-level production of the affinity matured antibody fragment.

### Example 13

### Integration and Mutation-of a Non-Coding Foreign Sequence

Modification of Clone 3 kb15a-7-4T

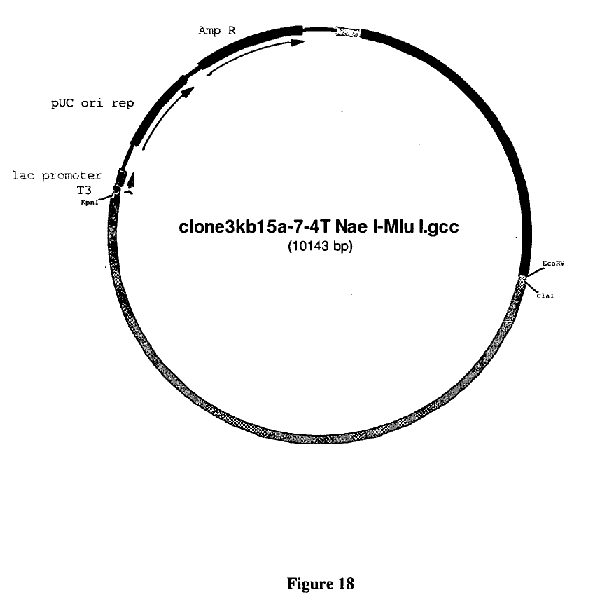

In order to linearise the 3 kb15a-7-4T construct at the 5 kb-3 kb junction using Cla I (NT 3586-3591), an additional Cla I site located in the multiple cloning site (MCS) was removed. The construct was digested with enzymes Mlu I and Nae I. Following digestion the ends were treated with Klenow (New England BioLabs Inc, USA) and religated using the T4 DNA ligase (Promega, USA). DNA was transformed into chemically competent Escherichia coli and incubated overnight. Bacterial colonies were subcultured and grown overnight at 37° C. DNA was extracted using the Qiaprep® Spin Miniprep Kit (Qiagen Inc., USA) according to the manufacturer's instructions and clones were screened by restriction enzyme digestion. The resulting construct will herein be referred to as 3 kb15a-7-4TΔ Mlu I-Nae I (FIG. 18) (SEQ ID NO:121).

Description of the Non-Coding Sequence for Integration into the Ig Heavy Chain Locus, Replacing the V Region (Rearranged VDJ)

The sequence spanning nucleotides 7905 and 330 of clone 3 kb15a-7-4TΔ Mlu I-Nae I will be integrated by homologous recombination and replace the V region in the heavy chain locus. This sequence will be referred to as the “foreign sequence” (SEQ ID NO:122). It contains the PUC origin of replication, which-was amplified for sequence identification and mutation analysis.

Determination of the Parental 3 kb15a-7-4TΔ Mlu I-Nae I Reference Sequence

To establish the parental nucleotide sequence in the PUC origin of replication, clone 3 kb15a-7-4TΔ Mlu I-Nae I was directly sequenced in five independent reactions using the forward primer 1066 (Table 8). These five sequences were aligned with five additional sequences of PCR products generated from the same region in clone 3 kb15a-7-4TΔ Mlu I-Nae I (SEQ ID NO:123). Alignment of these ten sequences generated a 1132 bp consensus sequence. This sequence was used to determine if mutations were present in sequences obtained from single cell PCR products.

Transfection of 3 kb15a-7-4TΔ Mlu I-Nae I into Different RAMOS Clones

Prior to transfection, the surface IgM status of two RAMOS clones; RA1 (ATCC, USA) and RA1 (George Klein, Karolinska Institute, Sweden) was determined. Cells were stained with anti-human IgM-FITC (see Example 8) and analysed on an Epics Elite flow cytometer (Beckman Coutler, USA).

IgM positive cells were transfected using Nucleofector technology (Amaxa, Germany) according to the manufacturer's instructions. Briefly, 2×10⁶ cells were centrifuged at 200×g for 10 minutes. The supernatant was removed and cells were resuspended in 100 μl of Solution V (Amaxa, Germany). Linearised plasmid 3 kb15a-7-4TΔ Mlu I-Nae I (2 μg) was added to the cell suspension. The cell suspension was transferred to Amaxa certified cuvettes and electroporated using the Nucleoporator, program O-17. Following electroporation, 500 μl of pre-warmed growth media (RPMI+10% FBS) was added to cells which were then transferred to a 12 well plate, containing 1.5 ml of medium per well. Cells were maintained in standard RAMOS culture conditions (see Example 4). Three independent experiments involving RAMOS clones RAI from ATCC and George Klein (GK) were performed (Table 7). Each experiment included four to five separate nucleofections.

TABLE 7
Description of three independent proof
of principal (POP) experiments

| | Experiment | | |
|---|---|---|---|
| | POP 1 | POP 2 | POP 3 |
| Source of RAMOS Clone RAI | ATCC | GK | ATCC |
| Duration in culture (days) | 27 | 29 | 21 |
| Number of cells | 2 × 10⁶ (×5) | 2 × 10⁶ (×4) | 2 × 10⁶ (×4) |

Enrichment of Integrants and Single Cell Sorting

IgM loss was used as a marker of integration of the foreign sequence (SEQ ID NO:122) into the Ig heavy chain locus, replacing the V region. In all three experiments, transfected cells were enriched using magnetic activated cell sorting (MACS®). Transfected cells were pooled, counted and then centrifuged at 300×g for 10 minutes. Cells (10⁷) were resuspended in 98 μl of buffer A (PBS, pH 7.2, 0.5% BSA, 2mM EDTA), incubated with 2 μl of anti-human IgM-FITC (see Example 8), mixed well and further incubated at 4° C. for 10 minutes in the dark. Next, cells were washed in 2 ml buffer A and centrifuged at 300×g for 10 minutes. Supernatant was removed and cells resuspended in 90 μl of buffer A. Anti-FITC MicroBeads (Miltenyi Biotech, Germany) (10μ) were added, mixed well and incubated for 15 minutes at 4° C. Cells were washed and resuspended in 500 μl of buffer A.

An LD column in a Midi MACS separator (Miltenyi Biotech, Germany) was used to sort cells. Firstly, the column was rinsed with 2 ml of buffer A. Next, 500 μl of cell suspension was applied to the column, followed by two 1 ml washes using buffer A. The flow through was collected which contained the IgM negative unlabeled fraction. The IgM positive labeled fraction was collected by removal of the column from the MACS separator and washing with 3 ml of buffer A. Both IgM negative and IgM positive fractions were centrifuged at 300×g, supernatant removed and cells were resuspended in 2 ml of growth media. Cells were then transferred to individual wells in a 6 well plate.

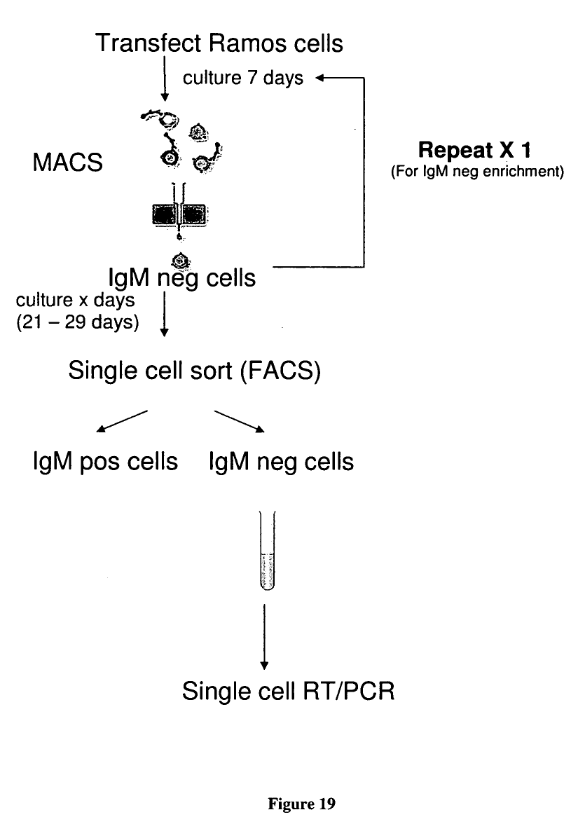

IgM negative and IgM positive populations were maintained separately in culture for various periods of time ranging from three to four weeks. Cell counts were performed on each population to monitor cell growth and viability before two subsequent enrichments were only performed on the IgM negative populations (FIG. 19). IgM negative cells were labeled and analysed by flow cytometry. Only live cells were selected for single cell sorting. Individual cells were sorted into 0.2 ml strip PCR tubes (Scientific Specialties Inc, USA) containing 4 μl of water using either a FACStar cytometer at Walter Elisa Hall Institute (WEHI, Australia) or a FACSVantage DiVa at the Australian Stem Cell Centre (Monash STRIP, Australia). As a control, single cells from the IgM positive populations were also sorted into strip PCR tubes. Cells were immediately frozen on dry ice and stored at −70 ° C.

Confirmation of Integration of the Foreign Sequence Replacing the V Region

Population of Cells

Initially, integration was demonstrated on IgM negative cell populations collected prior to single cell sorting and maintained in culture for 21 to 29 days. Genomic DNA was extracted from the cells using the GenoPrep™ kit (Scientifix, Australia) according to the manufacturer's instructions (see Example 1). This procedure was repeated for each population from all three experiments. Two sequential PCRs were performed on each sample. The first PCR reaction included; 2×Pfx amplification buffer, magnesium sulfate (1.5 mM), 1×PCR enhancer solution, forward primer (10 pM), reverse primer (10 pM), dNTPs (10 mM), genomic DNA (˜50 ng), platinum PfX DNA polymerase (1.25 U) and sterile water in a final volume of 50 μl. Cycling conditions were as follows; one cycle of 95° C. for 5 minutes, 25 cycles of 95° C. for 30 seconds, 60° C. for 30 seconds, 68° C. for two and a half minutes and one cycle of 72° C. for 7 minutes. A second PCR reaction was performed using nested primer sets. (Table 8) and 1.25 μl/50 μl of the first PCR as a template. Cycling conditions were the same as those used in the first PCR reaction.

TABLE 8

Primers used in single cell RT and PCR for screening
and sequencing

Primer

| | |
|---|---|
| Name | Sequence |

| | |
|---|---|
| 1068 | 5′ GAGGGTTAATTGCGCGCTTGGCGTAATCA 3′ |
| | (SEQ ID NO: 124) |
| 1069 | 5′ AGATGCTGAAGATCAGTTGGGTGCACGAGTG 3′ |
| | (SEQ ID NO: 125) |
| 1110 | 5′ GTTATCCGCTCACAATTCCACACAACATACGAGC 3′ |
| | (SEQ ID NO: 126) |
| 1111 | 5′ AGTTCTGCTATGTGGCGCGGTATTATCCCGTAT 3′ |
| | (SEQ ID NO:127) |
| 1066 | 5′ GGCCGCGTTGCTGGCG 3′ |
| | (SEQ ID NO:128) |
| 8606 | 5′ CCCAAGCTTTGCACACCCTAGGGTATGTTCTTGCAATTC 3′ |
| | (SEQ ID NO:51) |
| 8562 | 5′ CCGGAATTCAAAAAAATAAACTTGATTTATGATGGTCAA 3′ |
| | (SEQ ID NO:129) |
| 8603 | 5′ CCGGAATTCCGCGGTGACCTGCTTCCTGCCACCTGCTGT 3′ |
| | (SEQ ID NO:74) |

Products from the second PCR were run on a 1% agarose gel. For primers sets,(1068, 8562; 110, 8562) used to detect the 3′ junction a 4 kbp product of the expected size (3.8 kbp) was observed. Likewise, for primer. sets (8606, 1069; 8606, 1111) used to amplify the 5′ junction the expected 2.9 kbp product was observed.

Single Cells

To confirm that integration had occurred at the V region site, mRNA from single cells (also used for mutation analysis) was reverse transcribed as described below using the primer 8606. Two sequential PCRs were performed on the DNA using primers 8606, 1069 followed by 8606, 1111 for the 5′ junction and primers 1068, 8603 followed by 1110, 8603 for the 3′ junction. Products from the second PCR were run on a 1% agarose gel. For the 5′ junction an expected product of 2.9 kbp was observed. Likewise, for the 3′ junction, an expected product of 3.95 kbp was observed. Further confirmation of integration at both junctions was obtained by sequencing these PCR products.

Reverse Transcription on Single Cells

For cell lysis, 1 μl of an RNAse inhibitor (RNAseOut, Invitrogen) and 6 μl of a lysis buffer (50 mM KCl, 10 mM Tris-HCl pH 9.0, 0.1% Triton-X-100) was added to the frozen cell. The mixture was rapidly thawed, then frozen, three times before incubating at 94° C. for 2 minutes. The forward primer 1068 (2 pM) and dNTPs (10 mM each) were added to the lysate and heated at 65° C. for 5 minutes. The reactions were then placed on ice for 2 minutes.

Superscript III (Invitrogen, USA) was used to reverse transcribe the DNA into RNA. A cocktail mix containing, RNAseout (20 U), 1× reaction buffer, Superscript III (100 U) and nuclease free water in a volume of 7 μl, was added to the first reaction in a final volume of 20 μl. This reaction was then incubated at 55° C. for 60 minutes, followed by a 15 minute heat inactivation step at 70° C.

PCR on Single Cells

PCR was performed on single cells transfected with 3 kb15a-7-4TΔ Mlu I-Nae I from each of the three experiments described in Table 7 to obtain sequence information reflecting mutation rates.

Two sequential PCRs using nested primers sets 1068, 1069 and 1110, 1111 respectively (Table 8), were performed to amplify a fragment within the foreign integrated sequence (SEQ ID NO:122). The first reaction included; 2×Pfx amplification buffer, magnesium sulfate (1.5 mM), 1×PCR enhancer solution, forward primer (10 pM), reverse primer (10 pM), dNTPs (10 mM ), mRNA (2 μl/20 μl from RT reaction), platinum PfX DNA polymerase (1.25 U) and sterile water in a final volume of 50 μl. Cycling conditions were as follows; one cycle of 95° C. for 5 minutes, 25 cycles of 95° C. for 30 seconds, 60° C. for 30 seconds, 68° C. for two minutes and one cycle of 72° C. for 7 minutes. A second PCR reaction was performed using 1.25 μl/50 μl of the first PCR as a template to obtain sufficient product for sequencing.

PCR products from the second reaction (10 μl/50 μl) were run on a 1% agarose gel and visualized with ethidium bromide. The expected fragment size of 1.8 kbp was observed and used to screen for positive samples. DNA from these samples was purified and concentrated using standard methods for ethanol precipitation. Pellets were resuspended in sterile water to a final concentration of 40 ng/μl.

Sequencing PCR products

Sequencing reactions were only performed on the PCR positive samples. The reaction included; 1× reaction buffer, ABI Prism BigDye terminator mix (Applied Biosystems, USA), 40 ng of template DNA and. sterile water in a final volume of 20 μl. Cycle conditions were as follows; one cycle of 96° C. for 1 minute, 30 cycles of 96° C. for 10 seconds, 50° C. for 5 seconds, 60° C. for 4 minutes and one cycle of 10° C. for 2 minutes. Sequencing reactions were cleaned up using ethanol precipitation protocols including incubation at room temperature for 15 minutes followed by two ethanol washes Samples were then run on an Applied Biosystems 3730S genetic analyzer (Monash University, Micromon Sequencing facility, Australia).

Analysis of Sequences

The possibility of mutation artifacts introduced by either, Pfx platinum DNA polymerase (error rate 3×10⁻⁶) or AmpliTaq DNA polymerase (error rate 1.72×10⁻⁵), was eliminated by comparing five sequences directly from 3 kb145a-7-4Δ Mlu I-Nae I plasmid with five sequences of a PCR product generated from the same region in 3 kb15a-7-4Δ Mlu I-Nae I plasmid.

Comparisons of the consensus sequence (SEQ ID NO:123) with sequences from single cells transfected with 3 kb15a-7-4Δ Mlu I-Nae I were analysed. Point mutations were detected using BLAST, NBCI (http://www.ncbi.nlm.nih.gov/BLAST/) and confirmed by analyzing individual changes in chromatograms by three independent examiners. In total one hundred and fifteen sequences from transfected single cells in three independent experiments were analysed. Of these, forty nine sequences (42.61%) had one or more point mutations (Table 9). This is not surprising given that the selection process enriched for cell integrants and not for mutants of the foreign sequence.

TABLE 9
Mutations in sequences analysed

| | | |
|---|---|---|
| Mutated | Non mutated | Total |

| | | | | | | |
|---|---|---|---|---|---|---|
| POP 1 | 20 | (46.51%) | 23 | (53.49%) | 43 | (100%) |
| POP 2 | 14 | (31.82%) | 30 | (68.18%) | 44 | (100%) |
| POP 3 | 15 | (53.57%) | 13 | (46.43%) | 28 | (100%) |
| Total | 49 | (42.61%) | 66 | (57.39%) | 115 | (100%) |

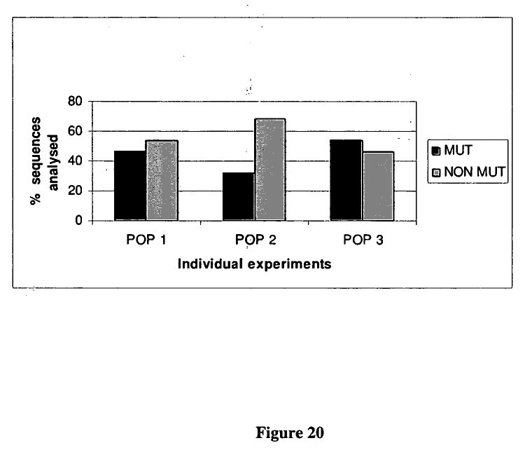

The frequency of mutated and non-mutated sequences varied between experiments (Table 9 and FIG. 20). Notably, the number of mutated sequences in POP 2 relative to POP 1 and POP 3 was significantly reduced. This most likely reflects the difference in RAI clones, given that POP 1 and POP 3 represent the RAI clone sourced from ATCC and POP 2 used RAI clone from George Klein Laboratory. Previous studies and our own experience supports differences in mutation rates between RAMOS clones. Interestingly, there were more mutated sequences in POP 3 which lasted 21 days in culture compared to POP 1 which was 27 days in culture. This supports a recent finding that mutations do not accumulate in culture over a long period of time.

The average mutation rate was calculated by dividing the total number of bases mutated by the total number of bases analysed from mutated sequences. The mutation rate, 1 in 412 bp is consistent with that quoted in the literature for RAMOS cells, 10⁻³ to 10⁻⁴ per bp per generation (four orders of magnitude higher than that observed at HPRT locus). In addition, all the mutations we observed were within 1648 bp of the promoter which is typical of somatic hypermutation (SHM) patterns observed in the immunoglobulin locus.

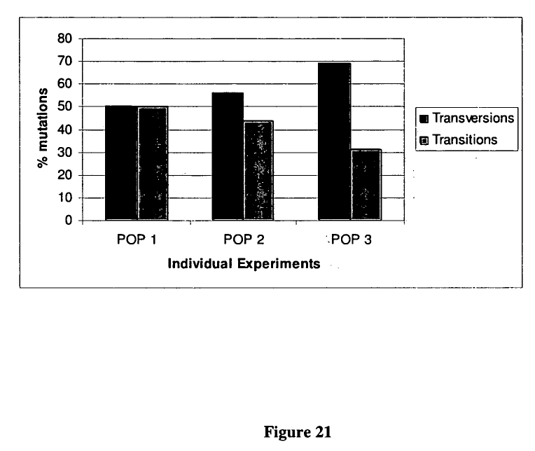

A total of one hundred and twenty one point mutations were detected. Of these sixty nine (57%) were transversions and fifty two (43%) were transitions. Hence, the ratio of transitions to transversions approximates 1:1. This ratio was reflected in POP 1 and POP 2, however in POP 3, the frequency of transversions was greater than that of transitions (Table 10 and FIG. 21).

TABLE 10
Frequency of transversions and transitions

| | | |
|---|---|---|
| Tranversions | Transitions | Total |

| | | | | | |
|---|---|---|---|---|---|
| POP 1 | 22 | (50%) | 22 | (50%) | 44 |
| POP 2 | 27 | (56.25%) | 21 | (43.75%) | 48 |
| POP 3 | 20 | (68.97%) | 9 | (31.03%) | 29 |
| Total | 69 | | 52 | | 121 |

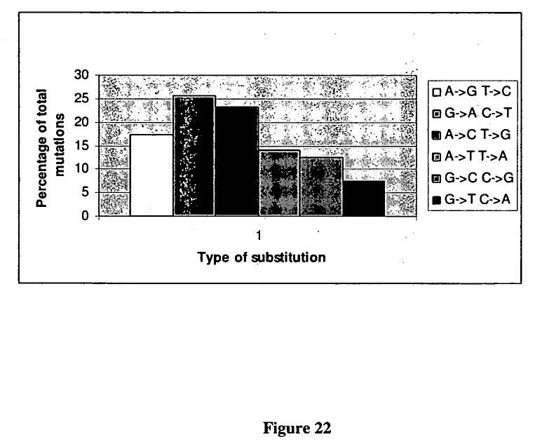

The percentage of mutations at CG pairs (the target for phase one of SHM) was 45.5% and at AT pairs (the second phase of SHM) was 54.5%. Analysis of individual base changes across the three data sets revealed that all possible nucleotide changes were detected (FIG. 22). Notably, an overall preference for G to A (21.5%) and Ttb G (15.7%) was observed (Table 11). The spectra of nucleotide substitutions from our data set supports that seen previously in the V gene from human B cells.

TABLE 11
Spectrum of nucleotide substitutions

| Overall | A | C | T | G | Total |
|---|---|---|---|---|---|
| A | — | 9 (7.44%) | 9 (7.44%) | 10 (8.26%) | 28 |
| C | 1 (0.83%) | — | 5 (4.13%) | 9 (7.44%) | 15 |
| T | 8 (6.61%) | 11 (9.09%) | — | 19 (15.70%) | 38 |
| G | 26 (21.49%) | 6 (4.96%) | 8 (6.61%) | — | 40 |
| Total | | | | | 121 |

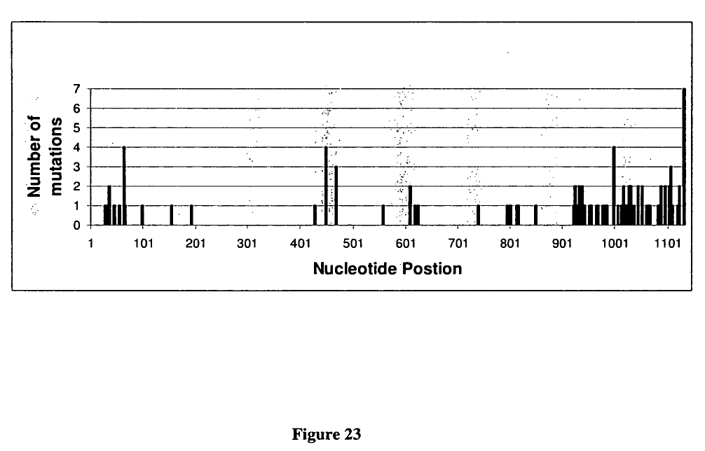

In our study, point mutations were not distributed equally throughout the sequence (FIG. 23). The majority of base changes were clustered towards the end of, the sequences analysed (positions 920 to 1132) which corresponds to 1436 bp to 1648 bp away from the promoter, whereas fewer mutations were clustered near the start of the sequence (position 28 to 192) only 545 bp to 709 bp from the promoter. In comparison to the mutation distribution in the V gene of humans, the majority of mutations occur within the complementary determining region (CDR3), which is 432 bp to 482 bp from the promoter. The remaining nucleotide substitutions were scattered throughout the middle of the sequence (position 448 to 850).

Of the one hundred and fifteen mutated sequences analysed, the majority of mutations occurred at a specific position only once. However, there were seven positions (64, 448, 468, 999, 1105, 1131, and 1132) at which base changes occurred in three or more sequences. Interestingly, with the exception of positions 1096 and 1132, all base substitutions which occurred more than once at a specific position, were the same nucleotide changes. At position 1096 there was a G to A (transition) in one sequence and a G to C (transversion) in another. Similarly, at position 1132, some sequences had T to G (transversion) but others had T to C (transition).

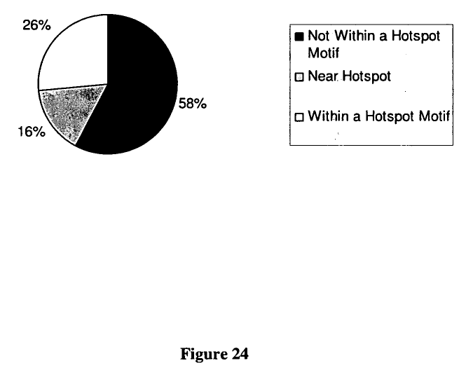

Surprisingly, 58% of the mutations did not occur within the classic hotspot motif RGYW and its complementary, WRCY (where R=purine, Y=pyrimidine and W=A or T). Conversely, 26% of base changes were within the RGYW hotspot and 16% occurred within five nucleotides of this motif (FIG. 24). These data indicate that mutations are not restricted to the classic hotspot motif and there must be other yet unidentified factors (either sequence or structure specific) which are susceptible to mutation.

### Example 14

### Surface Display of EGFP Using the Anchor Domain of CD26

The anchor domain of CD26 was added to the 3′ end of the gene for EGFP using sequential overlapping extension PCR and cloned into pME18s as previously described (see Example 9), except that additional restriction sites Age I and Nar I were added to the 5′ and 3′ ends of the EGFP gene respectively. This new construct was designated pME18sEGFPCD26 (SEQ ID NO:130).

To demonstrate surface expression of EGFPCD26, human embryonic kidney 293 T cells were transfected with 5 μg of pME18sEGFPCD26 or pME18sEGFP (control) using FuGENE6 as previously described (see Example 10). At 48 hours post transfection, cells were washed once with PBS and fixed (but not permeablised) with the addition of 1% formaldehyde in PBS for 10 minutes at 4° C. After fixation, the cells were washed with PBS and then anti-EGFP-Alexa555 (Molecular Probes, USA), diluted 1:100, was added for 45 minutes at 4° C. After staining, the cells were washed again in PBS and then analysed by fluorescence microscopy using an Olympus IX70 microscope and Olympus U-RFL-T burner.

Under green light (exciter BP510-550) cells expressing EGFPCD26 stained red and showed a distinct pattern characterized by bright red fluorescence around the periphery of the positive cells indicating the presence of the protein on the cell surface. In contrast, the cells expressing EGFP were not stained with the antibody indicating that the protein is confined to the cytoplasm of the cells. As a result, the CD26 anchor domain can be used in the methods of the invention to display expressed and mutated fusion proteins on the cell surface which allows the fusion proteins to be screened for a desired trait.

### Example 15

### Identification of the Rearranged VDJ Segment in Other Burkitt's Lymphoma Cell Lines

Three mammalian cell lines, Daudi, Raji (Jan Van Winkel, Genmab, The Netherlands) and Nalm-6 (Flinders Medical Centre, Australia) were obtained and cultured as previously described (see Example 4). Genomic DNA was extracted from cells using Genoprep DNA isolation Kit (Scientifix, Australia) according to manufacturer's instructions. The rearranged VDJ alleles were sequenced (see Example 1) and identified using V-Base (http://www.mrc-clpe.cam.ac.uk/vbase). Both Daudi and Nalm-6 cells typed as V₃₋₇₄ D₁₋₇ JH_{4b}, whereas Raji was V₄₋₃₄ D₃₋₁₆ JH_{4b}.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method for producing and selecting a gene product with desired characteristics, the method comprising
(i) introducing into a hypermutating cell a target nucleic acid molecule encoding a gene product such that the target nucleic acid molecule is integrated into an immunoglobulin locus of the genome of the hypermutating cell;
(ii) culturing the hypermutating cell such that the target nucleic acid molecule undergoes hypermutation during DNA and/or RNA synthesis, giving rise to a population of cells expressing mutant gene products; and
(iii) selecting a mutant gene product with desired characteristics.

2. A method as claimed in claim 1 wherein the immunoglobulin locus contains a rearranged V gene.

3. A method as claimed in claim 1 wherein the immunoglobulin locus contains a rearranged VH gene.

4. A method as claimed in claim 1 wherein the immunoglobulin locus contains the rearranged VH₄₋₃₄ allele.

5. A method as claimed in claim 1 wherein following integration of the target nucleic acid molecule into the immunoglobulin locus, the target nucleic acid molecule is operatively linked to a promoter.

6. A method as claimed in claim 5 wherein the promoter is an immunoglobulin heavy or light chain promoter.

7. A method as claimed in claim 5 wherein the promoter is endogenous to the hypermutating cell.

8. A method as claimed in claim 5 wherein the promoter is exogenous to the hypermutating cell.

9. A method as claimed in claim 5 wherein following integration the initiation codon of the target nucleic acid molecule is located within 2 kb of the 3′ end of the promoter.

10. A method as claimed in claim 5 wherein following integration the initiation codon of the target nucleic acid molecule is located within 500 bp of the 3′ end of the promoter.

11. A method as claimed in claim 5 wherein following integration the target nucleic acid molecule is located downstream of the promoter and upstream of an intronic enhancer with or without matrix attachment regions and/or 3′ enhancer.

12. A method as claimed in claim 1 wherein the target nucleic acid molecule is introduced into the cell by way of an integration vector comprising a sequence homologous to a region of at least 500 bp upstream of a rearranged V allele and a sequence homologous to a region of at least 500 bp downstream of a rearranged V gene.

13. A method as claimed in claim 1 wherein steps (ii) and (iii) are repeated.

14. A method as claimed in claim 1 wherein the method comprises a further step to increase the rate of mutation of the target nucleic acid molecule.

15. A method as claimed in claim 14 wherein the further step is to increase the levels of expression of activation-induced cytidine deaminase (AID) within the hypermutating cell.

16. A method as claimed in claim 1 wherein the mutant gene product is selected by way of an assay performed within the hypermutating cell.

17. A method as claimed in claim 16 wherein the assay performed within the hypermutating cell is a protein-fragment complementation assay (PCA).

18. A method as claimed in claim 1 wherein the target nucleic acid molecule is linked to a sequence encoding an anchor molecule such that following expression, the mutant gene product is displayed on the surface of the hypermutating cell.

19. A method as claimed in claim 18 wherein the mutant gene product is selected by detecting binding of a binding partner to the mutant gene product.

20. A method as claimed in claim 19 wherein the hypermutating cells are labelled with a detectable marker such as a fluorescent dye and the binding partner is immobilized.

21. A method as claimed in claim 19 wherein the binding partner is labelled with a fluorescent tag.

22. A method as claimed in claim 18 wherein hypermutating cell(s) displaying the mutant gene product bound to the labelled binding partner are sorted using a flow cytometric technique.

23. A method as claimed in claim 19 wherein the binding partner is selected from the group consisting of an antibody, receptor, transcription factor hormone, enzyme, cell surface molecule, DNA or RNA molecule.

24. A method as claimed in claim 1 which further comprises the step of recovering the target nucleic acid molecule encoding the selected mutant gene product.

25. A method as claimed in claim 24 wherein the recovery involves amplification of the polynucleotide by PCR or RT-PCR.

26. A method as claimed in claim 1 wherein the hypermutating cell is a mammalian, yeast, insect or bacterial cell.

27. A method as claimed in claim 26 wherein the hypermutating cell is a mammalian cell.

28. A method as claimed in claim 27 wherein the mammalian hypermutating cell is selected from the group consisting of RAMOS, BL2, BL41, BL70 and Nalm.

29. A gene product produced by a method as claimed in claim 1.

30. A vector for targeted integration into an immunoglobulin locus of a hypermutating cell, the vector comprising a sequence homologous to a region upstream of a rearranged V gene of the hypermutating cell, a sequence homologous to a region downstream of a rearranged V gene of the hypermutating cell and a site for integration of a target nucleic acid molecule.

31. A vector as claimed in claim 30 wherein the region upstream of the rearranged VH gene of the hypermutating cell is a region within nucleotides 1 to 5190 of SEQ ID NO:1.

32. A vector as claimed in claim 31 wherein the region is at least 500 bp within nucleotides 1 to 5190 of SEQ ID NO:1.

33. A vector as claimed in claim 31 wherein the region upstream of the rearranged VH gene of the hypermutating cell comprises nucleotides 191 to 5190 of SEQ ID NO:1.

34. A vector as claimed in claim 30 wherein the region downstream of the rearranged VH gene of the hypermutating cell is a region within nucleotides 5709 to 8699 of SEQ ID NO:1.

35. A vector as claimed in claim 30 wherein the downstream region is at least 500 bp within nucleotides 5709 to 8699 of SEQ ID NO:1.

36. A vector as claimed in claim 30 wherein the vector further comprises a selectable marker.

37. A vector for targeted integration comprising a sequence as set out in nucleotides 1 to 12990 of SEQ ID NO:110.

38. A vector as claimed in claim 30 wherein the vector further comprises a sequence encoding a signal and/or anchor molecule suitable for display of the gene product encoded by the target nucleic acid molecule.
